(19) United States
(12) Patent Application Publication
(10) Pub. No.: US 2022/0233688 A1
(43) Pub. Date: Jul. 28, 2022

(54) ADJUVANTS FOR IMMUNOGENIC COMPOSITIONS AND METHODS OF USE THEREOF

(71) Applicant: UNIVERSITY OF MARYLAND, BALTIMORE, Baltimore, MD (US)

(72) Inventors: Matthew Frieman, Baltimore, MD (US); Robert Haupt, Middletown, MD (US); Robert K. Ernst, Silver Spring, MD (US)

(21) Appl. No.: 17/616,584

(22) PCT Filed: Jun. 8, 2020
(86) PCT No.: PCT/US2020/036685

§ 371 Date: Dec. 3, 2021

Related U.S. Application Data
(60) Provisional application No. 62/858,034, filed on Jun. 6, 2019.

(51) Int. Cl. A61K 39/39; A61K 39/145; A61P 31/16; A61P 31/20

(52) U.S. Cl. CPC A61K 39/39; A61K 39/145; A61K 2039/55572; A61P 31/20; A61P 31/16

(57) ABSTRACT

The present invention provides pharmaceutical compositions capable of inducing an immune response in a subject, comprising an effective amount of a viral immunogen and an adjuvant, wherein the adjuvant comprises a lipid A mimetic. Methods for inducing an immune response are also provided.

### CROSS-REFERENCE TO RELATED APPLICATIONS

This application claims the benefit of U.S. Provisional Appl. No. 62/858,034, filed Jun. 6, 2019, the contents of which are hereby incorporated by reference in their entirety.

### FIELD OF THE INVENTION

The invention relates to infectious disease, in particular to vaccines for preventing viral disease and methods for immunizing against viral disease.

### BACKGROUND OF THE INVENTION

Microbial and viral infections continue to be a leading cause of morbidity and mortality in humans worldwide (Krammer, F. et al. Influenza. Nat Rev Dis Primers 4, 3, doi:10.1038/s41572-018-0002-y (2018)). Of those, Influenza viruses, enveloped viruses of the orthomyxovirus family of RNA viruses, infect a wide variety of species and remain a worldwide public health threat through seasonal epidemics which result in high levels of morbidity and mortality in humans, and economic loss (Palese et al. Nat Med 10, 882-87, doi:10.1038/nm1141 (2004); Wiwanitkit et al. J Biomed Res 27, 339, doi: 10. 75555BR.27 0.20130087 (2013); Storms et al. Influenza Other Respir Viruses 7, 1328-1335 doi:10.1 11 1/irv.12106 (2013); Colizza et al. PLoS medicine 4, e13, doi: 10.1371/journal.pmed.0040013 (2007); Taubenberger et al. Cell host & microbe 7, 440-451, doi:10.1016/j.chom.2010.05.009 (2010); Molinari et al. Vaccine, 2007. 25(27): p. 5086-96). Influenza virus causes a respiratory illness with symptoms of fever, cough, runny nose, headaches, muscle and body aches, and often gastrointestinal symptoms. Influenza outbreaks can occur when influenza A viruses with novel antigenicity emerge and spread in a population with little preexisting immunity (Suarez et al. Emerging infectious diseases 10, 693-699, doi:10.3201/eid1004.030396 (2004); Treanor et al. The New England journal of medicine 350, 218-220, doi:10.1056/NEJMp038238 (2004); Suarez et al. Emerg Infect Dis, 2004. 10(4): p. 693-9; Treanor, J. et al. N Engl J Med, 2004. 350(3): p. 218-20). This can happen in both developed and underdeveloped countries. Recently in the 2018-2019 influenza season in the United States alone there were more than 35.5 million illnesses, 490,000 hospitalizations and 34,000 deaths with over twice as many hospitalizations and deaths in the previous flu season (Xu et al. MMWR Morb Mortal Wkly Rep, 2019 68(24): p. 544-551). Worldwide, the virus causes 3-5 million severe cases with 500,000 deaths every year.

In addition to normal seasonal infections, Influenza viruses have a history of pandemic outbreaks causing significant morbidity and mortality (e.g., 1918 Spanish influenza, 1957 Asian influenza, 1968 Hong Kong influenza and the 2009 swine origin influenza outbreak) (Taubenberger et al. Emerging infectious diseases 12, 15-22, (2006); Reid, et al. Laboratory investigation; a journal of technical methods and pathology 79, 95-101 (1999); Taubenberger et al. Emerg Infect Dis, 2006. 12(1): p. 15-22; Reid et al. Lab Invest, 1999. 79(2): p. 95-101). To reduce the threat of both seasonal and novel pandemic Influenza virus outbreaks, an effective vaccine that produces high levels of protection across antigenically divergent strains and in populations prone to increased morbidity and mortality is required (Miller et al. Cell 157, 294-299 (2014); Surichan et al. Vaccine 29 Suppl 1, A29-33, doi: 10.1016/j.vaccine.2011.04.120 (2011); Stephenson et al. Lancet Infect Dis 4, 499-508, doi: 10.1016/S 1473-3099(04)01105-3 (2004).

Worldwide surveillance efforts are coordinated by the World Health Organization (WHO) to inform vaccine design, with the current circulating strains being Influenza A virus subtypes H1N1 pdm09 and H3N2, as well as the Influenza B variants (Yamagata and Victoria lineages) (WHO.org). In addition to the seasonal strains, the emergence of pandemic strains is of great concern. A pandemic strain is caused by the emergence of a virus with an HA protein for which the majority of humans do not have immunity. This situation occurred in 2009 when the circulating strain of Influenza H1N1 appeared for which most adults did not have cross-reactive antibodies. Seasonal antigenic drift of the HA protein produces novel antigenic sites that when divergent enough, are not seen by the host immune response as they have seen HA antigens previously, and thus the viruses are able to cause increased disease. The development of safe and effective vaccines that offer complete protection across divergent Influenza virus strains has been a goal of scientists and public health officials (Kilbourne et al. Nat Med 5 (1999); Butler et al. Nature 560, 158-160, doi:10.1038/d41586-018-05889-1 (2018)). The current vaccine platforms approved for use in humans (inactivated, live attenuated and recombinant HA vaccines) are all produced using the WHO data to match the antigenicity of circulating virus strains. The surveillance data is increasingly robust, and modeling studies are working to predict the antigenic changes to occur in future strains, however all are hampered by the reduced efficacy of the current platforms, whether in immune-competent individuals or in immune-deficient individuals (both infant and elderly). New adjuvant formulations with current Influenza vaccine platforms provide the possibility of enhanced immunogenicity and protection from not only homotypic strains but heterotypic strains.

It is an annual challenge to determine the strains of circulating Influenza virus that will be prevalent in an upcoming season. Shifts in strains are largely caused by the antigenic drift mutations in the immunodominant hemagglutinin (HA), and to a lesser extent the neuraminidase (NA) surface glycoproteins (Nelson et al. Nat Rev Genet, 2007. 8(3): p. 196-205). The Influenza vaccine most prevalent in use today is a standard-dose quadrivalent influenza shot for people aged six months and older, manufactured using virus grown in eggs (Wiley et al. Nursing, 2018. 48(11): p. 11-13; Campos-Outcalt et al. J Farm Pract, 2018. 67(9): p. 550-553). For people 65 and older, there is a vaccine available (FLUAD®) that is formulated with a proprietary adjuvant known as MF59; the antigen in FLUAD® is also manufactured using an egg-based process (Fluad—an adjuvanted seasonal influenza vaccine for older adults. Med Lett Drugs Ther, 2016. 58(1486): p. 8). MF59 is an oil-in-water emulsion of squalene oil which helps create a more potent and durable immune response to the vaccination (Cruz-Valdez et al. Hum Vaccin Immunother, 2018. 14(2): p. 386-395). The use of an adjuvant also allows for antigen sparing which creates a greater supply of vaccines, improves seroprotection against drifted strains, and helps promote heterologous cross protection against divergent strains of Influenza (Ko et al. Hum Vaccin Immunother, 2018. 14(12): p. 3041-3045.; Ansaldi et al. Vaccine, 2008. 26(12): p. 1525-9.; Ko et al. Antiviral Res, 2018. 156: p. 107-115).

For next generation vaccines to be developed, it is critical that high quality antigenic targets, such as Influenza hemagglutinin (HA) protein, be used in combination with an adjuvant to increase their immunogenic capacities. To date, vaccine adjuvants have been developed using a trial-and-error approach (Pittman et al. Vaccine 14, 337-343 (1996)). Adjuvants licensed for use in human vaccines, such as Alum (Alhydrogel) and oil-in-water emulsions, cannot be used universally and have drawbacks including the requirement for repeated application and the inappropriate skewing of certain innate and adaptive immune responses. For example, Alum (Alhydrogel) has been shown to stimulate T-helper (Th) 2-based IgG1 and IgE antibody production in mice, which is effective at combating extracellular pathogens. Alum does not induce strong cell-mediated immunity or complement fixing and virus-neutralizing Th 1 type antibodies IgG2a and IgG2b (Tan et al. J Viral 88, 13580-13592, doi:10.1128/JV1.02289-14 (2014); Hiatt et al. Proc Natl Acad Sci USA 111 (2014)). To date, the mechanisms resulting in this response are still poorly understood. Oil-in-water emulsions, as with Alum, elicit a Th2-based immune response, but they do not provide strong, long lasting protection against intracellular pathogens. In contrast, the Toll-like receptor (TLR) 4 agonists 3-O-deacyl-4-monophosphoryl lipid A (MPL, GlaxoSmithKline) and aminoalkyl glucosaminide phosphates (AGPs) are well studied examples of TLR4 ligand adjuvants that can promote a Th1 (cellular)-biased immune response, which is effective at combating intracellular pathogens.

To overcome these limitations, future vaccines will require novel, rationally-designed adjuvants that stimulate through defined components of the host innate immune systems, specifically the TLRs. TLRs are pattern recognition receptors (PRR) which detect molecules that are broadly shared by pathogens but distinct from host molecules. By binding a ligand, such as a purposefully modified lipid A (the membrane anchor of lipopolysaccharide (LPS) and a TLR4 agonist), TLR4 can initiate innate immune responses and the development of antigen-specific acquired immunity. The level of TLR4 activation instructs the type of adaptive immune response that arises from vaccination. The panel of cytokines that are differentially produced can result in the skewing of the adaptive immune response toward Th1, which is most effective at combating intracellular infections, toward Th2 and Th9, which are most useful during extracellular infections, toward T-regulatory cells (Treg), which suppress immune responses, or toward Th17, which provides immunity at epithelial/mucosal barriers. Currently, the TLR4 agonists MPL and aminoalkyl glucosaminide phosphates (AGPs) are being used as stand-alone immunotherapeutic adjuvants or formulated with Alum to potentiate host responses through the innate and adaptive immune systems (Casella et al. Cellular and molecular life sciences: CMLS 65, 3231-3240 (2008)). However, due to the product heterogeneity arising from the chemical synthesis of MPL and the labor-intensive nature of synthesizing AGPs, alternatives are required for synthesizing the next generation of TLR4 agonists.

RSV is a negative sense single stranded RNA virus that is very easily spread from human to human through direct contact. Most children are infected with RSV before age two and present with cold-like symptoms for up to three weeks. However, in very young or immunocompromised patients, serious symptoms such as bronchiolitis and pneumonia can occur and lead to hospitalization or, rarely, death. Despite over fifty years of attempting to create a vaccine for this debilitating infection, there is still no effective RSV vaccine available. Most recently efforts have been placed into the development of a pre-fusion form of the antigenic F protein (F0), which, is described in more detail below and, we propose to develop in these studies.

The RSV virion consists of a nucleoprotein (N) wrapped around the viral RNA forming a ribonucleoprotein (RNP) complex. The immunodominant antigen on the surface of the virion is a glycoprotein called F, and is responsible for fusion of the viral envelope with the plasma membrane. Antibodies to F are able to block entry of the virus by blocking the interaction between F and the membrane. The F protein is initially in an inactive prefusion precursor state (F0) that is post-translationally cleaved to generate a fusion competent F protein (F1). Standard production of F for vaccine experiments results in an F protein in the post-fusion cleaved state (F1). The use of the F1 form in vaccination experiments results in only moderate neutralizing antibody production against RSV virions because most antibodies are targeting the post-fusion F1 structure, which is not present on the surface of circulating virions.

Prevention of HPV-etiologic oral and anogenital cancers through broad and consistent HPV vaccination programs has achieved a great degree of success. Current vaccines are based on noninfectious VLPs of the major L1 capsid protein that contain no nucleic acid material. Bivalent and quadrivalent versions of these vaccines have been FDA-approved and have succeeded in reducing infection and subsequent CIN by 70-80% for the HPV16/18 types, responsible for the majority of HPV-related cancers. However, the HPV type coverage by the most recently FDA-approved nonavalent vaccine, Gardasil-9, only extends protection to 90% of HPV-positive cancers (Serrano et al. Infect Agent Cancer. 7:38). Further addition of new L1-VLP species to the nonavalent vaccine would continue to increase manufacturing cost-of-goods (COG) with only small incremental improvements in protection coverage over the general population. Therefore, an HPV vaccine that eliminates the need for further increasing the number of L1-VLP populations while further extending coverage through advantaging a new type of vaccine platform would be beneficial.

HPV infection occurs through viral engagement of heparin sulfate proteoglycans and laminin on the surface of basal keratinocytes, leading to internalization of virions into the endosomal pathway (Nguyen et al. Curr Probl Dermatol. 45:19). Following major L1 capsid disassembly in the late endosome, the minor capsid protein L2 is exposed and cleaved by the host-derived convertase furin, required for endosomal escape of the viral genome to the cell nucleus. A key challenge for the further development of optimized HPV-L1-based vaccines is the requirement to continuously add new L1-VLP populations derived from additional HPV types, neutralization of HPV through L1-specific responses being type-specific, a cost-magnifying endeavor due to a lack of sufficient homology between L1 sequences. The L2 capsid demonstrates a much higher rate of sequence homology compared to L1, and antibodies specific to the N-terminal HPV16-L2 region have demonstrated cross-neutralization activity directed against other HPV subtypes, a phenomenon not achievable with the L1 capsid subunit, in which cross-neutralization between HPV L1 sequences is not observed (Jagu et al. 2013, Phylogenetic considerations in designing a broadly protective multimeric L2 vaccine, J Virol. 87:6127; Roden et al. 2000; Virology, 270:254; Roden et al. 1996, J Virol. 70:5875.; Chen et al. 2000, Mol Cell. 5:557; El Aliani et al. 2020, Gene. 747:144682) In particular, the L2 amino acid-region 17-36 has a high degree of conservancy between subtypes and is named RG1 due to recognition by the RG1 mAb (Gambhira et al. 2007, J Virol. 81:13927).

Vaccine configurations that take advantage of the L2 epitope have included linear L2 epitope repeats on a modified human IgG1 Fc scaffold, a concatenated fusion protein adjuvanted with Montanide ISA51, or as adenovirus- or AAV vector-expressed L2 epitopes (Chen et al. PLoS One. 9:e95448; Motavalli et al. J Immunol Res 2018:9464186; Vujadinovic et al. Vaccine, 36:4462.; Nieto et al. PLoS One 7:e39741; Jiang et al. Expert Rev Vaccines. 15:853). These L2 vaccines have demonstrated cross-neutralizing antibody responses against HPV of diverse types including HPV5/6/11/16/18/31/33/45/52/58, underlining the cross-neutralization potential of L2 (Chen et al. 2014 PLoS One. 9:e95448.; Nieto et al. 2012. PLoS One. 7:e39741). To take advantage of the naturally immunogenic nature of an L2 receptive array while retaining the well-established potency of the L1 subunit, a VLP vaccine was engineered to express the RG1 HPV16-L2 epitope inserted within the DE loop of each of 72 HPV16-L1 capsid subunits, which spontaneously assemble into VLPs (Schellenbacher et al. J Virol. 83:10085). This RG1-VLP vaccine adjuvanted with aluminum salts has demonstrated robust immunity in mice and the induction of cross-neutralization to many mucosal high-risk HPV types, including HPV18 and HPV39 (Schellenbacher et al. 2013, J Invest Dermatol. 133:2706). However, Gardasil, adjuvanted only with aluminum hydroxyphosphate sulfate, has demonstrated deficiencies in % seropositivity and magnitude of IgG titers and the generation of neutralizing Abs to non-vaccine HPV types (HPV31/45) in human subjects compared to Cervarix, which employs a combination adjuvant consisting of Alhydrogel and the TLR4 agonist monophosphoryl lipid A (MPLA) (Nicoli et al. 2020. Vaccines (Basel); Mariz et al. 2020. NPJ Vaccines. 5:14).

For next generation vaccines to be developed, there is a critical need in the art for high-quality antigenic targets, such as Influenza rHA protein, to be used in combination with an adjuvant of the right design to increase the immunogenic capacities of the vaccines and to shorten the vaccine production timescale by eliminating time consuming egg-based manufacturing of the immunogen.

### SUMMARY OF THE INVENTION

It is to be understood that both the foregoing general description of the embodiments and the following detailed description are exemplary, and thus do not restrict the scope of the embodiments.

The present invention provides new adjuvants prepared by a Bacterial Enzymatic Combinatorial Chemistry (BECC) process that can enhance the quantity and quality of antibodies raised in a subject mammal to an antigen co-formulated in the subject vaccine. As shown herein, influenza recombinant haemagglutinin (rHA) vaccines comprising the presently disclosed BECC adjuvants have demonstrated enhanced protection in multiple mouse, protection and virus infection models in comparison with more conventional vaccines.

Provided herein is an evaluation of leading adjuvant candidates, BECC438, BECC470-12 and BECC470-16 (FIG. 2), for optimal formulation and efficacy, immunogenicity, antigen sparing, dose sparing, toxicity, and immune correlates of protection when formulated with Influenza HA. In contrast to the highly proinflammatory E. coli lipid A, BECC438 (bisphosphorylated) and BECC 470-12 (monophosphorylated) are hepta-acylated structures with three fatty acids (2-C16, 1-C12 for BECC470) attached to three of the four 3-OH C14 acyl groups on the diglucosamine backbone. These significant alterations affect the ability of this molecule to interact with components of the host innate immune system, specifically the MD-2/TLR4 receptor complex. PHAD (Avanti Polar Lipids) is a synthetically made monophosphorylated hexa-acylated lipid A molecule that partially resembles Glaxo Smith-Kline (GSK) MPL. In contrast to the synthetically produced PHAD, GSK MPL is a biological derivative of Salmonella minnesota LPS and contains a mixture of multiple acylated lipid A structures.

The three selected BECC structures have been preliminarily evaluated for efficacy, immunogenicity, and protection using HA protein, as described below. In some aspects, the present invention is based on adjuvanted influenza virus vaccine enhancement with BECC adjuvants. The ability of test vaccines to reduce effective levels of antigen and adjuvant in normal and elderly mice demonstrates the advantage of the BECC adjuvants compared to Alum and PHAD.

It is an object of the present invention to discover and test appropriate adjuvants that can be paired with immunogens, such as Influenza rHA protein, to make vaccines that more effectively protect mammal subjects, including humans, than has heretofore been possible, wherein the vaccines can be manufactured on the shortest practical timescale.

In one aspect, the invention provides a pharmaceutical composition capable of inducing an immune response in a subject, comprising an effective amount of a viral immunogen and an adjuvant, wherein the adjuvant comprises a lipid A mimetic molecule selected from the group consisting of:

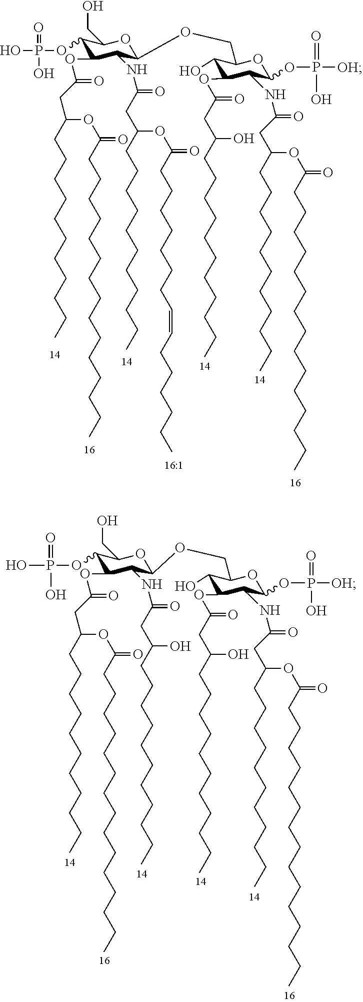

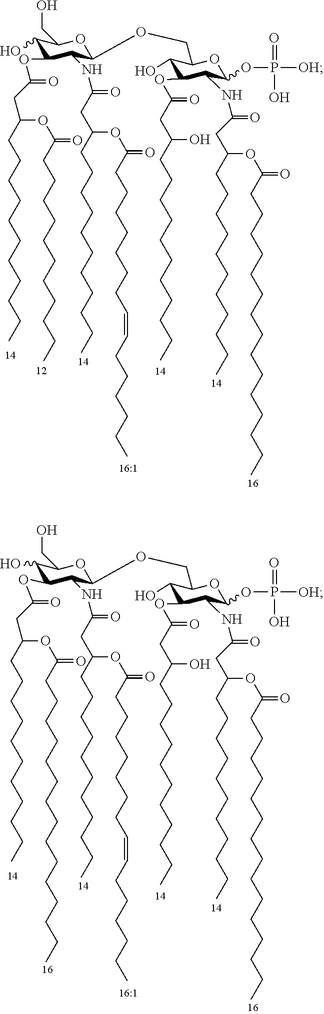

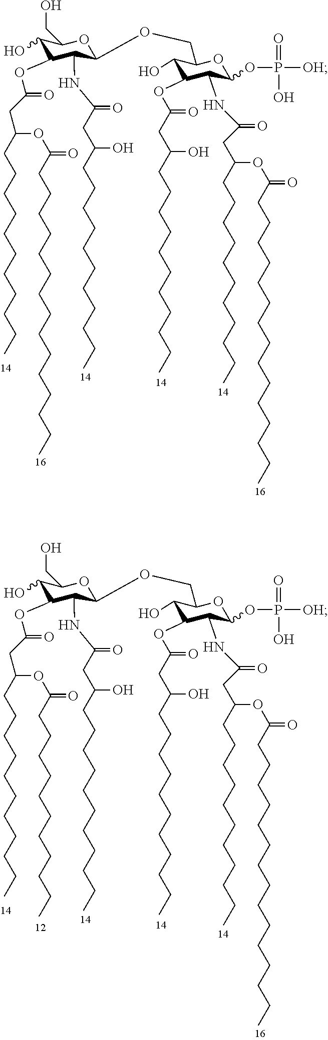

and combinations thereof.

In some embodiments, the subject is a human.

In some embodiments, the viral immunogen is from an influenza virus. In some embodiments, the viral immunogen is from an influenza A and/or B virus.

In some embodiments, the viral immunogen is from a virus selected from the group consisting of an orthomyxovirus, a coronavirus, a respiratory syncytial virus (RSV), or a human papillomavirus (HPV).

In some embodiments, the viral immunogen comprises a polypeptide antigen or an antigenic fragment thereof. In some embodiments, the antigen comprises influenza hemagglutinin (HA) protein or an antigenic fragment thereof.

In another aspect, the invention provides a method of inducing an immune response in a subject, comprising administering to the subject a pharmaceutical composition comprising an effective amount of a viral immunogen and an adjuvant, wherein the adjuvant comprises a lipid A mimetic molecule selected from the group consisting of:

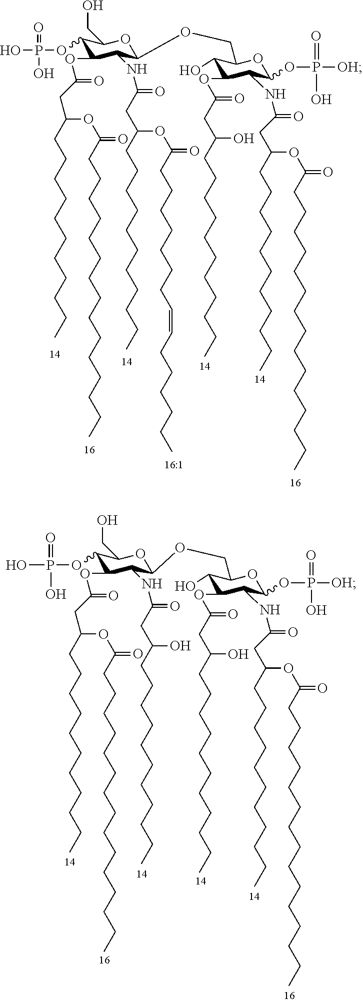

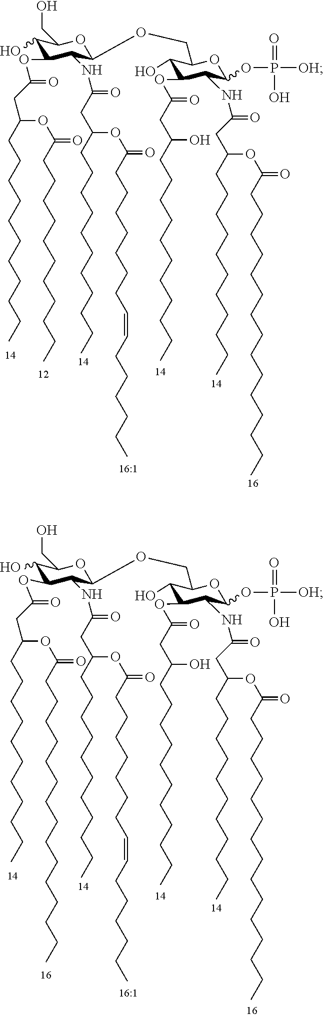

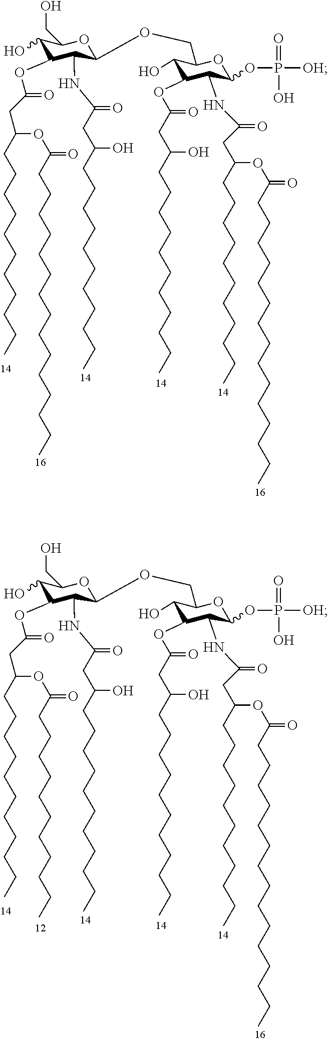

and combinations thereof. In some embodiments, the subject is a mammal. In some embodiments, the subject is a human. In some embodiments, the viral immunogen is from an influenza A and/or B virus. In some embodiments, the viral immunogen comprises hemagglutinin or any antigenic fragment thereof. In some embodiments, the subject is a human. In some embodiments, the subject is 55 years old or greater. In some embodiments, the subject is a human 55 years old or greater, the adjuvant is BECC470 and the immunogen, e.g., rHA, is for influenza.

In some embodiments, the viral immunogen is from an orthomyxovirus, a coronavirus, a respiratory syncytial virus (RSV), or a human papillomavirus (HPV). In some embodiments, the pharmaceutical composition is administered by intramuscular injection. In some embodiments, the immunogen is selected from the group consisting of a split virus, a subunit antigen, an inactivated whole virus, a live attenuated virus, and combinations thereof.

In some embodiments, the subject is administered the composition once. In some embodiments, the subject is administered a first dose of the composition as a prime, followed by administration of one or more additional boost administrations.

In some embodiments, the method reduces lethality of a secondary bacterial infection.

In some embodiments, the effective amount of the viral immunogen administered is from about 50 ng to about 1.0 mg per kg of body weight of the subject. In some embodiments, the effective amount of the viral immunogen administered is from about 15 μg to about 1.9 mg per kg of body weight of the subject.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The skilled artisan will understand that the drawings, described below, are for illustration purposes only. The drawings are not intended to limit the scope of the present teachings in any way.

FIG. 1. Bacterial Enzyme Combinatorial Chemistry (BEGG). BEGG can be used to create novel lipid A mimetics for therapeutic use. Briefly, avirulent Yersinia pestis lipid A is modified genomically or transformed with a plasmid to express lipid A biosynthesis enzymes from a variety of bacterial backgrounds. These strains are cultured and the lipooligosaccharide is purified.

FIG. 2. Lipid A structures of the highly proinflammatory E. coli, known TLR4 ligand phosphorylated hexa-acyl disaccharide/glucopyranosyl lipid adjuvant (PHAD/GLA), and the preferred adjuvant molecules of the present invention, BECC438 and BECC470 (BECC470-12 and BECC470-16 versions). Red boxes indicate major modifications to E. coli structures.

FIG. 3. Activation of primary mouse splenocytes by BECC438 and BECC470. Primary mouse splenocytes from BALB/c mice were incubated for 36 hours with 1 μg/ml of E. coli lipopolysaccharide (LYS) PHAD or the BECC. Cytokine secretion data, as measured by Luminex assay, is shown in pg/ml.

FIG. 4. Activation of primary human peripheral blood mononuclear cells (PBMCs) by BECCs. Primary human PBMCs from BALB/c mice were incubated for 36 hours with 1 μg/ml of E. coli LPS, PHAD, or the BECC. Cytokine secretion data, as measured by Luminex assay, is shown in pg/ml.

FIG. 5. Upregulation of co-stimulatory surface markers on primary human monocyte derived dendritic cells upon BECC470 lipid A stimulation. Primary monocyte derived dendritic cells from four separate donors were cultured with 1000 ng/ml of E. coli LPS, PHAD, or BECC470. Surface marker expression, CD80. CD83, and CD40 is graphed as mean fluorescence intensity (MB) as measured by flow cytometry.

FIG. 6. Weight loss curve of 10-week-old BALB/c mice infected with NL/09 at various concentrations.

FIG. 7. Weight loss and antibody responses after Influenza virus NL/09 challenge of HA vaccinated mice.

FIG. 8. ELISA titers of HA protein alone vaccination.

FIG. 9. Weight loss after vaccination and Influenza NL/09 challenge.

FIG. 10. Secondary challenge with S. pneumoniae causes lethal disease following Influenza virus challenge. A. Survival curve with either Influenza (PR8) infection alone or in combination with SP3. B. Histology of either mock, or virus infected. C. Colony-forming unit (CFU) counts on lung, blood and spleen from mock or infected mice.

FIG. 11. Determining Cal/09 HA antigen dose needed in vaccination for protection from NL/09 Influenza A infection. Seven day weight loss in BALB/c mice (3 per group) after infection with 3200 plaque-forming units (PFU) of NL/09 with either A) prime only vaccination schedule or B) prime+boost schedule. C) Pre-infection serum total IgG antibody titer in prime only and prime+boost vaccination group (averaged) with D) corresponding area under the curve (averaged). E) Virus titer of lung homogenate 7-days post infection from prime only and prime+boost groups with F) pathology inflammation scoring of lung histology slides (averaged).

FIG. 12. Homologous challenge protection. A) Pre-infection day 28 serum ELISA total IgG, B) isotype specific IgG2a and C) IgG1 in prime+boost vaccination schedule. Area under the curve (AUC) group average of above serum ELISA curves for D) total IgG, E) IgG2a and F) IgG1. G) 7-day weight loss in BALB/c mice (5 per group) after infection with 3200 PFU of NL/09. H) Virus titer of lung homogenate 7-days post infection with I) average inflammation pathology scoring of lung histology slides.

FIG. 13. Adjuvant dose sparing. A) Pre-infection day 28 serum ELISA total IgG with 50, 5 or 0.5 μg of BECC438 or BECC470 adjuvants. B) Area under the curve (AUC) group average of ELISA curve in (A) for total IgG antibody. C) 7-day weight loss in BALB/c mice (5 per group) after infection with 3200 PFU of NL/09. D) Virus titer of lung homogenate 7-days post infection with E) pathology inflammation scoring of lung histology slides (averaged).

FIG. 14. RSV F pre-fusion stabilized protein. Cartoon of the structure of RSV-F mutant with mutated amino acids that lock the F protein into a prefusion F0 state. Note this protein maintains all noted antigenic sites for neutralizing antibody targeting. (Adapted from McLellan et al, Science 2013).

FIG. 15. RSV neutralizing antibody titers. Serum was collected on day 35 from mice that were immunized on day 0 and 21 with either PBS, WT RSV A2, RSV F alone, or RSV F with GLA/SE, PHAD, or BECC438. Neutralizing antibodies titers were measured using an ELISA based microneutralization assay, Log 2 IC50 values are graphed showing mean±SD.

FIG. 16. BECC438 induces a balanced IgG1/IgG2a response. Serum was collected on day 35 from mice that were immunized on day 0 and 21 with either PBS, WT RSV A2, RSV F alone, or RSV F with GLA/SE, PHAD, or BECC438. Antibody isotype levels were measured using an ELISA, serum antibody concentrations are graphed showing mean±SD.

FIG. 17. BECC438+RSV F confers complete protection from challenge. Lung tissue was collected four days after challenge (day 39) from mice that were immunized on day 0 and 21 with either PBS, WT RSV A2, RSV F alone, or RSV F with GLA/SE, PHAD, or BECC438. PFU/g tissue are graphed with mean±SD.

FIG. 18. BECC screening pipeline flowchart

FIG. 19. Lipid A structures for highly pro-inflammatory E. coli, known adjuvant PHAD, and the molecule under investigation in this proposal BECC438.

FIG. 20. Activation primary mouse splenocytes by BECC438. Primary mouse splenocytes from C57BL6 and BALBc strains were incubated for 36 hours with 1000 ng/mL of E. coli LPS, BECC438, or PHAD. Cytokine secretion data, as measured by Luminex assay, is shown in pg/mL.

FIG. 21. Activation of human primary PBMC by BECC438. Primary human PBMC from three separate donors were incubated for 36 hours with 1000 ng/mL of E. coli LPS, BECC438, or PHAD. Cytokine secretion data, as measured by Luminex assay, is shown in pg/mL.

FIG. 22. Upregulation of co-stimulatory surface markers on primary human monocyte derived dendritic cells upon BECC438 lipid A stimulation. Primary monocyte derived dendritic cells from four separate donors were cultured with 1000 ng/mL of E. coli LPS, BECC438, or PHAD. Surface marker expression, CD80, CD83, and CD40, is graphed as mean fluorescence intensity (MFI) as measured by flow cytometry.

FIG. 23. Cell culture stimulation of primary cells ex vivo. (A) Splenocytes from either BALB/c or C57BL6 mice were stimulated with 1000 ng/mL of E. coli LPS, BECC470, BECC438, or PHAD. Supernatants were collected from cultures 48 h later and cytokine secretion measured using Luminex assay. (B) PBMC from three separate normal human donors were used to generate monocyte-derived dendritic cells which were then stimulated with 10 ng/mL of E. coli LPS, BECC470, BECC438, or PHAD. Upregulation of costimulatory markers were measured via flow cytometry after 24 h stimulation and percent positivity is shown separately for the three donors tested.

FIG. 24. Chemical structures of BECC470 and BECC438. Both molecules are hexa-acylated with one modification in the acyl-chain arrangement. BECC470 has a secondary 12 C addition at the 3′ position while BECC438 has a secondary 16:1 C addition at the 2′ position. The 4′ phosphate group is removed in BECC470 making it mono-phosphorylated while BECC438 remains bis-phosphorylated.

FIG. 25. Enhancement of RG1-VLP-specific humoral immunity in the presence of BECC compounds+Alhydrogel. Mice were immunized i.m. with 2 μg RG1-VLP alone or adjuvanted with 50 μg alum+/−25, 50 μg PHAD, 25, 50 μg BECC438, 25, 50 μg BECC470, or Gardasil-9 on days 0, 14, 28 and peripheral blood sera samples derived on day 42. (A-B) Sera samples were tested for HPV16-L1- and HPV16-L2 RG1-specific IgG via ELISA. (C-F) Sera samples were analyzed for neutralizing titers via fc-PBNA specific for PsV16-, PsV18-, PsV39-, PsV6-SEAP. (F) Numbers of responding mice with detectable titers of PsV6-neutralizing activity are indicated. Statistical comparisons were generated using one-way ANOVA nonparametric analysis with the Kruskal-Wallis multiple comparisons test. p<0.05 was considered significant; ns, not significant; p >0.05; *, p<0.05; **, p<0.01, ***, p<0.001, ****, p<0.0001.

FIG. 26. Activity of BECC compounds relies on Alhydrogel as adjuvant vehicle. Mice were immunized i.m. with 2 μg RG1-VLP alone or adjuvanted with 50 μg alum+/−25 μg 3D-PHAD, 25 μg BECC438, 25 μg BECC470, or Gardasil-9 on days 0, 21, 42 and peripheral blood sera samples derived on day 56. Sera samples were tested for HPV16-L1- and HPV16-L2 RG1-specific IgG via ELISA. Statistical comparisons were generated using one-way ANOVA nonparametric analysis with the Kruskal-Wallis multiple comparisons test. p<0.05 was considered significant; ns, not significant; p>0.05; *, p<0.05; **, p<0.01, ****, p<0.0001.

FIG. 27. BECC compounds+Alhydrogel accelerate the appearance of L1/L2 Ab levels as well as HPV16/18-neutralization titers. Mice were immunized i.m. with 2 μg RG1-VLP alone or adjuvanted with 50 μg alum+/−25 μg PHAD, 25 μg BECC438, 25 μg BECC470, or Gardasil-9 on days 0, 14, 28 and peripheral blood sera samples derived on days 14, 28, 42. (A-B) Sera samples were tested for HPV16-L1- and HPV16-L2 RG1-specific IgG via ELISA. (C-D) Day 14/28 sera samples were analyzed for neutralizing titers via fc-PBNA specific for PsV16-, PsV18-SEAP. Statistical comparisons were generated using one-way ANOVA nonparametric analysis with the Kruskal-Wallis multiple comparisons test. p<0.05 was considered significant; ns, not significant; p>0.05; *, p<0.05; **, p<0.01, ***, p<0.001, ****, p<0.0001.

FIG. 28. Immunization with reduced VLP doses achieves optimal L1/L2 Ab levels and HPV-neutralizing titers when adjuvanted with Alhydrogel/BECC470. Mice were immunized i.m. with 0.5/1/2 μg RG1-VLP alone or adjuvanted with 50 μg alum+/−25 μg BECC470, or Gardasil-9 on days 0, 14, 28 and peripheral blood sera samples derived on days 14, 28, 42. (A-B) Sera samples were tested for HPV16-L1- and HPV16-L2 RG1-specific IgG via ELISA. (C-F) Day 42 sera samples were analyzed for neutralizing titers via fc-PBNA specific for PsV16-, PsV18-, PsV39-, PsV6-SEAP. Statistical comparisons were generated using one-way ANOVA nonparametric analysis with the Kruskal-Wallis multiple comparisons test. p<0.05 was considered significant; ns, not significant; p>0.05; *, p<0.05; **, p<0.01, ***, p<0.001, ****, p<0.0001.

FIG. 29. Induction of robust IFN-γ responses after BECC compound supplementation of Alhydrogel. Mice were immunized i.m. with 2 μg RG1-VLP alone or adjuvanted with 50 μg alum+/−25, 50 μg PHAD, 25, 50 μg BECC438, 25, 50 μg BECC470, or Gardasil-9 on days 0, 21, 42 and peripheral blood sera samples derived on day 56. Spleens were harvested on day 56 and splenocytes restimulated in vitro with HPV16-L1 VLPs for 48 h an SFUs (spot-forming units)/1e6 cells analyzed via ELISPOT. Statistical comparisons were generated using one-way ANOVA nonparametric analysis with the Kruskal-Wallis multiple comparisons test. p<0.05 was considered significant; ns, not significant; p>0.05; *, p<0.05; **, p<0.01, ***, p<0.001.

FIG. 30. T cell responses in Alhydrogel/BECC-vaccinated mice are boosted by 4^{th} immunization 3 months later. Mice were immunized i.m. with 2 μg RG1-VLP adjuvanted with 50 μg alum+/−25 μg BECC470, or Gardasil-9 on days 0, 14, 28. Some mice were sacrificed on day 42 and splenocytes analyzed for IFN-g SFUs/1e6 cells in response to HPV16-L1 VLPs via ELISPOT. Separate groups of mice immunized identically with VLPs+alum or VLPs+alum/BECC470 were maintained until day 126, when they received a 4^{th} immunization and 7 days later, splenocytes were tested via ELISPOT. Statistical comparisons were generated using one-way ANOVA nonparametric analysis with the Kruskal-Wallis multiple comparisons test. p<0.05 was considered significant; ns, not significant; p>0.05; *, p<0.05; **, p<0.01, ***, p<0.001.

FIG. 31. BECC470 induces T follicular helper cells in lymphoid compartments as well as a sustained elevation of the L1 Ab response over several months. Mice were immunized i.m. with 2 μg RG1-VLP adjuvanted with 50 μg alum+/−25 μg BECC470, or Gardasil-9 on days 0, 14, 28 and peripheral blood sera samples derived on days 14, 28, 42, 70, 98, 125. Some mice were sacrificed on day 42 and splenocytes and popliteal LN cells were analyzed for Tfh content via FACS. (A-B) Sera samples were tested for HPV16-L1-specific IgG via ELISA. (B-C) Splenocytes and popliteal LNs were stained for presence of CD4⁺ CXCR5⁺ PD-1⁺Tfh cells. Statistical comparisons were generated using one-way ANOVA nonparametric analysis with the Kruskal-Wallis multiple comparisons test. p<0.05 was considered significant; ns, not significant; p>0.05; *, p<0.05; **, p<0.01, ***, p<0.001.

FIG. 32. Agonist vs Antagonist Activity of BECC Molecules.

FIG. 33. Influenza antigens.

FIG. 34. Intranasal challenge of Balb/C mice PFU Needed to Cause Disease. 6-8 week old mice, BALB/c.

FIG. 35. Evaluation of Influenza HA protein. 6-8 week old mice.

FIG. 36. Evaluation of BECC Adjuvants Alone. 6-8 week old mice.

FIG. 37. Evaluation of BECC Adjuvants. 6-8 week old mice.

FIG. 38. Influenza virus HA based vaccine. 6-8 week old mice.

FIG. 39. Homologous challenge with Influenza NL/09. 6-8 week old mice.

FIG. 40. Homologous challenge with Influenza NL/09. 6-8 week old mice.

FIG. 41. Homologous challenge with Influenza NL/09. 6-8 week old mice.

FIG. 42. Homologous challenge with Influenza NL/09. 6-8 week old mice.

FIG. 43. Homologous challenge with Influenza NL/09. 6-8 week old mice.

FIG. 44. Homologous challenge with Influenza NL/09. 6-8 week old mice.

FIG. 45. Prime Only Vaccination with Influenza NL/09. 6-8 week old mice.

FIG. 46. Prime Only Vaccination with Influenza NL/09. 6-8 week old mice.

FIG. 47. Prime Only Vaccination with Influenza NL/09. 6-8 week old mice.

FIG. 48. Prime Only Vaccination with Influenza NL/09. 6-8 week old mice.

FIG. 49. Prime Only Vaccination with Influenza NL/09. 6-8 week old mice.

FIG. 50. Heterologous challenge with Influenza Sing/15. 6-8 week old mice.

FIG. 51. Heterologous challenge with Influenza Sing/15. 6-8 week old mice.

FIG. 52. Heterologous challenge with Influenza Sing/15. 6-8 week old mice.

FIG. 53. Homologous challenge with Influenza NL/09. Elderly Mice.

FIG. 54. Intranasal challenge of Balb/C mice. PFU Needed to Cause Disease in Elderly Mice.

FIG. 55. Homologous challenge with Influenza NL/09. Elderly Mice. >11 month old mice.

FIG. 56. Homologous challenge with Influenza NL/09. Elderly Mice. >11 month old mice.

FIG. 57. Homologous challenge with Influenza NL/09. Elderly Mice. >11 month old mice.

FIG. 58. Homologous challenge with Influenza NL/09. Elderly Mice. >11 month old mice.

FIG. 59. Homologous challenge with Influenza NL/09.Elderly Mice. >11 month old mice.

FIG. 60. Homologous challenge with Influenza NL/09. Elderly Mice. >11 month old mice.

FIG. 61. Homologous challenge with Influenza NL/09. Elderly mice.

FIG. 62. Homologous challenge with Influenza NL/09. 6-8 week old mice.

FIG. 63. Antigen Sparing Study.

FIG. 64. Heterologous Challenge Study.

FIG. 65. Prime only single vaccination. a) Pre-infection day 28 serum ELISA total IgG with 5, 10 or 15 ug HA protein in combination with 50 or 100 ug of BECC470 adjuvant in prime only schedule (averaged). b) Area under the curve (AUC) group average of ELISA curves for total IgG antibody. c) 7-day weight loss in BALB/c mice (5 per group) after infection with 3200 PFU of NL/09. d) Virus titer of lung homogenate 7-days post infection with e) pathology inflammation scoring of lung histology slides (averaged).

FIG. 66. Protection from heterologous Sing/2015 Influenza A challenge. a) Pre-infection day 28 serum ELISA total IgG in prime+boost schedule (averaged). b) Area under the curve (AUC) group average of serum ELISA curves for total IgG. c) 7-day weight loss in BALB/c mice (5 per group) after infection with 51 PFU of Sing/2015. d) Day 28 serum antibody viral neutralization assay. e) Virus titer of lung homogenate 7-days post infection with f) pathology inflammation scoring of lung histology slides (averaged).

### DETAILED DESCRIPTION OF THE INVENTION

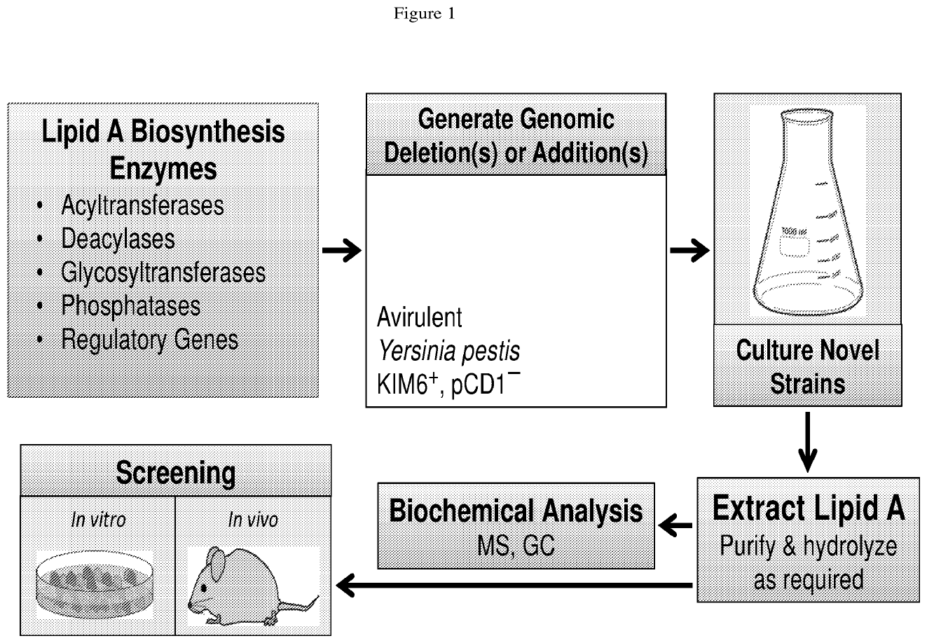

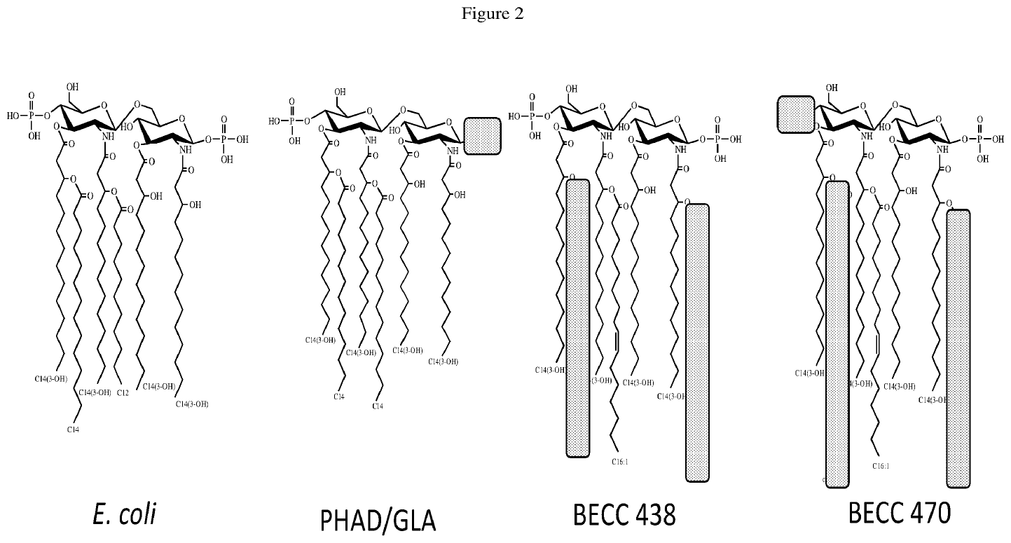

Provided herein are immunogenic pharmaceutical compositions prepared by combining immunogens with next generation vaccine adjuvants that can be efficiently manufactured using simple biosynthesis mechanisms that stimulate specific components of an immune response. A proven biosynthetic method, bacterial enzymatic combinatorial chemistry (BECC), is an alternative synthetic route that can be used to produce lipid A mimetic adjuvants quickly and efficiently, and BECC offers the advantage of ease of manipulation of immunostimulatory properties to facilitate the choice of a final adjuvant molecule (FIG. 1) (Gregg, K. A. et al. Rationally Designed TLR4 Ligands for Vaccine Adjuvant Discovery. mBio 8, doi:10.1128/mBio.00492-17 2017)). BECC technology can be used to rapidly generate novel lipid A structures as Toll-like Receptor 4 ligands (TLR4Ls) for use as adjuvants.[15] BECC involves expression or deletion of enzymes, including acyltransferase, deacylase, and/or phosphatase, from the lipid A synthesis pathways in Gram-negative bacteria, allowing for creation of unique lipid A-based structures and direct isolation/purification of TLR4Ls from a bacterial pellet without requiring further modification. See FIG. 1. To date, over 70 BECC derived Toll-like Receptor 4 (TLR4) interacting molecules have been generated, and all have been structurally characterized using mass spectrometry and gas chromatography. These BECC molecules were then entered into an in vitro vaccine adjuvant screening pipeline that measures the ability to stimulate innate immune signaling, elicit cytokine secretion in both mouse and human primary cell lines, and upregulate surface costimulatory markers on human monocyte derived dendritic cells (DC) (Gregg, K. A. et al. Rationally Designed TLR4 Ligands for Vaccine Adjuvant Discovery. mBio 8, doi:10.1128/mBio.00492-17 (2017)).

In the examples of the present invention described herein, BECC lipid A mimetic TLR4Ls BECC438, BECC470-12 and BECC470-16 (shown below) are studied to demonstrate their adjuvant potential in a prime-boost or prime only vaccination schedule and to compare the adjuvant properties of these molecules to the known adjuvants alum (alhydrogel) and phosphorylated hexa-acyl disaccharide (PHAD). As used herein, the symbol “BECC470” can refer either to either molecules BECC470-12 or to BECC470-16 as provided herein. Alhydrogel is an aluminum hydroxide wet gel suspension which induces a strong Th2 immune response. While not wishing to be bound by any particular theory, it is thought that alum adjuvants work by improving the attraction and uptake of antigen by antigen-presenting cells (APCs), and allowing for extended release of antigen through a ‘depot’ effect. (Martinon, S., et al., J Immunol Res, 2019. 2019: p. 3974127.; Hem et al., Pharm Biotechnol, 1995 6: p. 249-76.; Ghimire, T. R., Springerplus, 2015. 4: p. 181). Recently, TLR4Ls have been used as adjuvants that stimulate stronger Th1 immune responses through pattern recognition receptors (PRR), (Gao, J. and Z. Guo, Med Res Rev, 2018. 38(2): p. 556-601). The TLR4L PHAD, a synthetic monophosphoryl Lipid A (MPLA), is effective at inducing a Th1 immune response (Hernandez, et al., Crit Care Med, 2019. 47(11): p. e930-e938.; Gregg, K. A., et al., Vaccine, 2018. 36(28): p. 4023-4031). BECC adjuvants, screened using reporter cell lines and flow cytometry for the ability to activate NFκB and cytokine production, are capable of stimulating an innate immune response greater than that generated by PHAD but less than that generated by the pyrogenic E. coli lipopolysaccharides (LPS). In a Yersinia pestis rF1V protein vaccination model, C57BL6 mice had balanced IgG1 and IgG2c levels with protection from lethal E pestis infection when adjuvanted with BECC438. (Gregg, K. A., et al., Vaccine, 2018. 36(28): p. 4023-4031) Similar to other TLR4Ls, BECC adjuvants are capable of driving a more balanced Th1/Th2 immune response than does Alum alone. BECC molecules are also are capable of being manufactured reproducibly, inexpensively, and in large quantities, making them advantageous for development as adjuvants for large scale vaccines such as those needed for influenza.

Reference will now be made in detail to the presently preferred embodiments of the invention which, together with the drawings and the following examples, serve to explain the principles of the invention. These embodiments are described in sufficient detail to enable those skilled in the art to practice the invention, and it is understood that other embodiments may be utilized, and that structural, biological, and chemical changes may be made without departing from the spirit and scope of the present invention.

Unless defined otherwise, all technical and scientific terms used herein have the same meanings as commonly understood by one of ordinary skill in the art.

In some embodiments, the practice of the present invention employs various techniques of molecular biology (including recombinant techniques), microbiology, cell biology, biochemistry and immunology. See, e.g., Sambrook et al. Molecular Cloning: A Laboratory Manual, 2^{nd} edition (1989); Current Protocols in Molecular Biology (F. M. Ausubel et al. eds. (1987)); the series Methods in Enzymology (Academic Press, Inc.); PCR: A Practical Approach (M. MacPherson et al. IRL Press at Oxford University Press (1991)); PCR 2: A Practical Approach (M. J. MacPherson, B. D. Hames and G. R. Taylor eds. (1995)); Antibodies, A Laboratory Manual (Harlow and Lane eds. (1988)); Using Antibodies, A Laboratory Manual (Harlow and Lane eds. (1999)); and Animal Cell Culture (R. I. Freshney ed. (1987)).

Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341).

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “an antibody” includes a plurality of antibodies, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” means plus or minus 10% of the numerical value of the number with which it is being used.

In one embodiment, the invention provides a pharmaceutical composition capable of inducing an immune response in a subject, comprising an effective amount of a viral immunogen and an adjuvant, wherein the adjuvant comprises a lipid A mimetic molecule selected from the group consisting of:

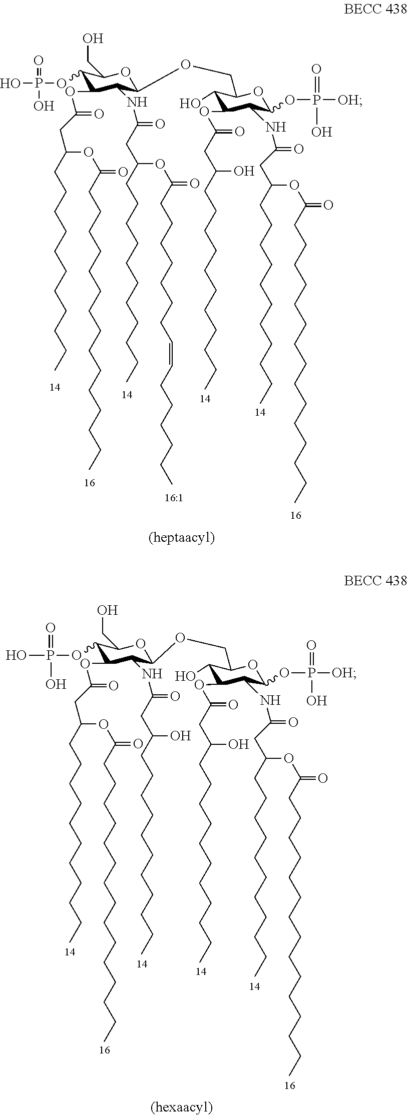

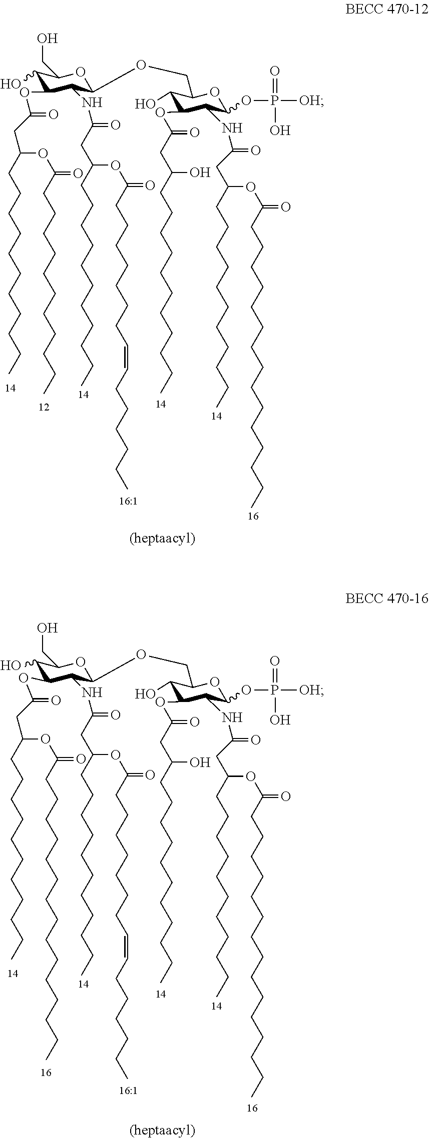

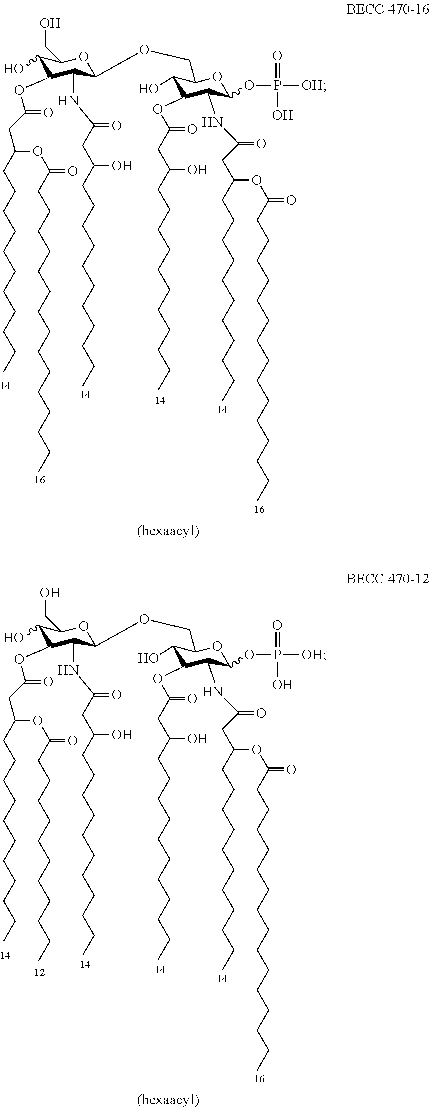

and combinations thereof.

In another embodiment, the invention provides a method of inducing an immune response in a subject, comprising administering to the subject a pharmaceutical composition comprising an effective amount of a viral immunogen and an adjuvant, wherein the adjuvant comprises a lipid A mimetic molecule selected from the group consisting of:

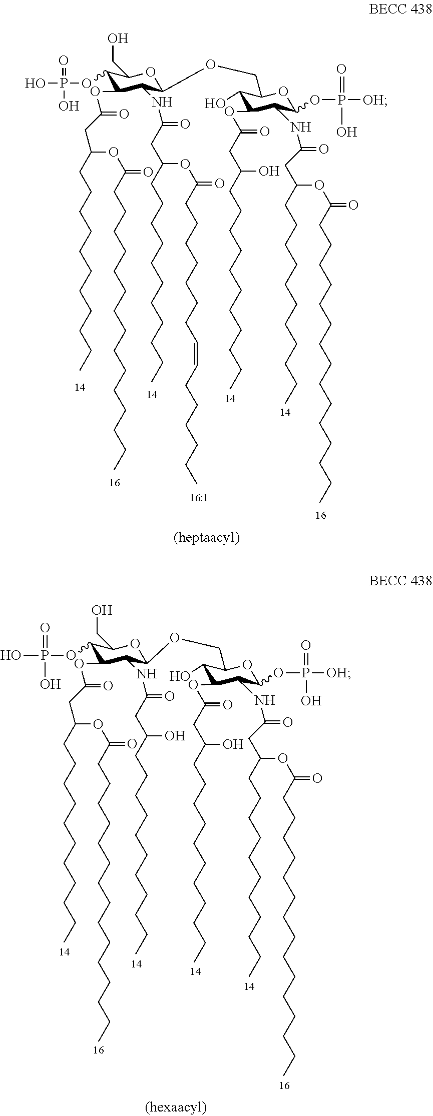

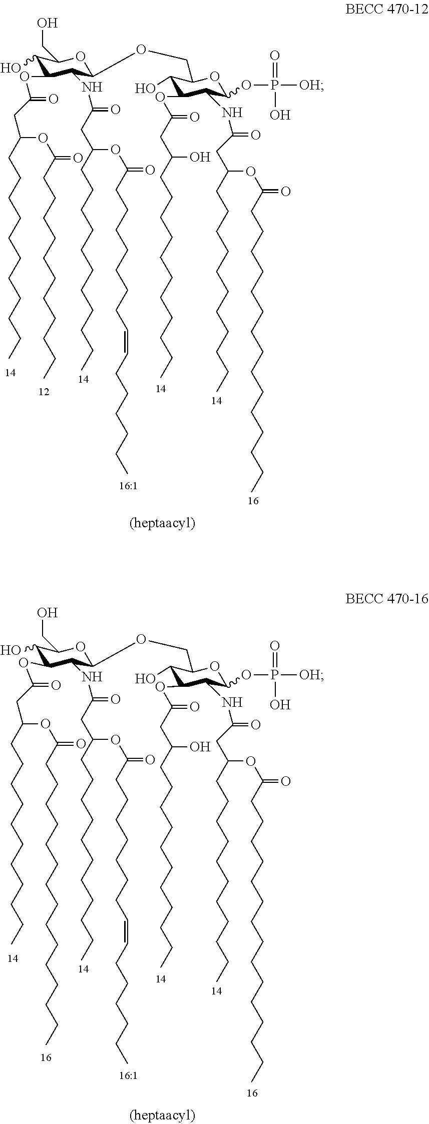

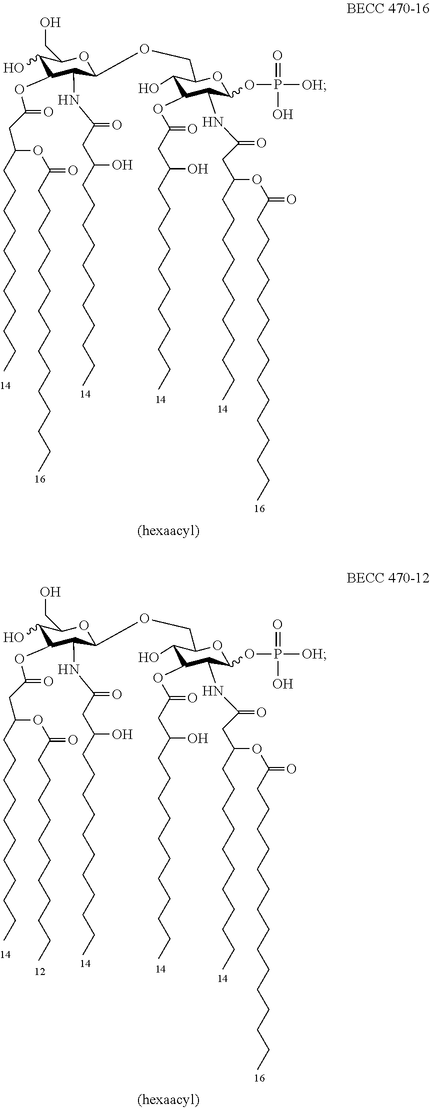

and combinations thereof.

The adjuvant compounds above are “lipid A mimetics.” As used herein “lipid A mimetics” refer to compounds that act on Toll-like receptor 4 (TLR4) complex and can be useful as an adjuvant in a vaccine for the prevention of viral disease in a subject, either alone or in combination with other therapies.

Pharmaceutically acceptable derivatives, such as prodrugs, and salts of the above-mentioned compounds are also contemplated by the present disclosure. The form of the lipid A derivative adjuvant is not particularly limiting. For example, the lipid A derivative adjuvant can be in the form of a pharmaceutically acceptable salt. Mixtures of different forms, and compositions that include mixtures of forms are possible. In some embodiments, the lipid A derivative adjuvant(s) may be provided as salts with pharmaceutically compatible counterions. Pharmaceutically compatible salts may be formed with many organic or inorganic acids, including but not limited to hydrochloric, sulfuric, acetic, lactic, tartaric, malic, succinic, etc.; or bases. Non-limiting examples of pharmaceutically acceptable salts include sodium, potassium, lithium, calcium, magnesium, iron, zinc, hydrochloride, hydrobromide, hydroiodide, acetate, citrate, tartrate and maleate salts, and the like. Combinations of different salt forms are possible.

The lipid A mimetic can be prepared by any suitable method. U.S. Pat. No. 10,358,667 and U.S. Patent Appl. Pub. No. 2020/0121705 (incorporated by reference herein) provide approaches of using Bacterial Enzymatic Combinatorial Chemistry (BECC) to make rationally-designed lipid A structures by modifying the lipid A structure of a lipopolysaccharide (LPS) or lipooligosaccharide (LOS) from a Gram negative bacteria (such as an attenuated (BSL-2 approved) Yersinia pestis (Yp) strain). In general aspects, this approach uses the lipid A structure present in LPS/LOS synthesized in bacteria as a lead molecule or structure to be modified by heterologous in trans expression of lipid A biosynthesis enzymes. These enzymes are obtained from a wide variety of bacterial backgrounds with specificities for the removal or addition of fatty acid chain, phosphates moieties, and carbohydrates to the lipid A backbone. In particular aspects, one approach uses the non-stimulatory, hypoacylated, and bisphosphorylated lipid A structure present in LOS synthesized by a Yp strain. One can test the immunotherapeutic use of these new molecules in vitro and in vivo to identify novel molecules representing adjuvants and/or immunomodulating reagents. One can also include well-characterized immunostimulants, such as MPL and known LPS structures, as comparisons to the molecules synthesized by BECC. The protective innate/adaptive immune responses by this novel approach of creating new adjuvants has important implications at least in the fields of antigen recognition, formulation, and vaccine design. In some embodiments, the bacteria that produces the lipid A mimetics is an Archaebacteria. In specific embodiments, the bacteria is an extremophile, including an Acidophile; Alkaliphile; Anaerobe; Cryptoendolith; Halophile; Hyperthermophile; Hypolith; Lithoautotroph; Metallotolerant; Oligotroph; Osmophile; Piezophile; Polyextremophile; Psychrophile/Cryophile; Radioresistant; Thermoacidophile; or Xerophile, for example.

In some embodiments, the bacteria that produces the lipid A mimetics is a Gram-negative bacteria. In some embodiments, the Gram-negative bacteria may be of any kind, including from Acetobacter, Borrelia, Bordetella, Burkholderia, Campylobacter, Chlamydia, Enterobacter, Eshcerichia, Fusobacterium, Helicobacter, hemophilus, Klebsiella, Legionella, Leptospiria, Neisseria, Nitrobacter, Proteus, Pseudomonas, Ricketsia, Salmonella, Serratia, Shigella, Thiobacter, Treponema, Vibrio, or Yersinia. In specific embodiments, one or more of the following bacteria are utilized to produce the lipid A (ASLA) based therapeutic: Acetic acid bacteria, Acinetobacter baumannii, Agrobacterium tumefaciens, Anaerobiospirillum, Arcobacter, Arcobacter skirrowii, Armatimonas rosea, Bacteroides, Bacteroides fragilis, Bacteroides ruber, Bartonella taylorii, Bdellovibrio, Brachyspira, Cardiobacterium hominis, Chthonomonas calidirosea, Coxiella burnetii, Cyanobacteria, Cytophaga, Dialister, Enterobacter, Enterobacter cloacae, Enterobacter cowanii, Enterobacteriaceae, Enterobacteriales, Escherichia, Escherichia coli, Escherichia fergusonii, Fimbriimonas ginsengisoli, Fusobacterium necrophorum, Fusobacterium nucleatum, Fusobacterium polymorphum, Haemophilus haemolyticus, Haemophilus influenzae, Helicobacter, Helicobacter pylor, Klebsiella pneumoniae, Legionella, Legionella pneumophila, Leptotrichia buccalis, Escherichia coli, Luteimonas aestuarii, Luteimonas aquatica, Luteimonas composti, Luteimonas lutimaris, Luteimonas marina, Luteimonas mephitis, Luteimonas vadosa, Megamonas, Megasphaera, Meiothermus, Methylobacterium fujisawaense, Morax-Axenfeld diplobacilli, Moraxella, Moraxella bovis, Moraxella osloensis, Morganella morganii, Negativicutes, Neisseria cinerea, Neisseria gonorrhoeae, Neisseria meningitidis, Neisseria sicca, Nitrosomonas eutropha, Nitrosomonas halophila, Nitrosomonas oligotropha, Pectinatus, Pelosinus, Pontiac fever, Propionispora, Proteobacteria, Proteus mirabilis, Proteus penneri, Pseudomonas, Pseudomonas aeruginosa, Pseudomonas, Pseudomonas luteola, Pseudoxanthomonas broegbernensis, Pseudoxanthomonas japonensis, Rickettsia rickettsii, Salmonella, Salmonella bongori, Salmonella enterica, Salmonella enterica subsp. enterica, Selenomonadales, Serratia marcescens, Shigella, Sorangium cellulosum, Sphaerotilus, Spirochaeta, Spirochaetaceae, Sporomusa, Stenotrophomonas, Stenotrophomonas nitritireducens, Thermotoga neapolitana, Trimeric autotransporter adhesin, Vampirococcus, Verminephrobacter, Vibrio adaptatus, Vibrio azasii, Vibrio campbellii, Vibrio cholerae, Vitreoscilla, Wolbachia, or Zymophilus.

In some embodiments, the lipid A mimetic adjuvants are produced by Gram-negative microorganism Yersinia pestis. In particular aspects, the bacteria in which the lipooligosaccharide/lipid A-based mimetics are generated is an avirulent Y. pestis strain, such as one that has lost one or more virulence plasmids. In some embodiments, the Gram-negative microorganism is wild-type Yersinia pestis KIM6 strain, although any number of the modified KIM6 strains may be employed (e.g., KIM6 del PhoP (regulator) could be made with LpxF+(expressing a phosphatase) or KIM6 del LpxD (acyltransferase) could be made with a del PmrK (which would not add aminoarabinose).

In some embodiments, the Gram-negative microorganism is engineered to express one or more non-endogenous lipid A biosynthetic enzymes, inactivate/delete one or more endogenous lipid A biosynthetic enzymes, modify one or more endogenous lipid A biosynthetic enzymes, and/or increase or decrease expression of one or more endogenous lipid A biosynthetic enzymes. In some embodiments, the lipid A biosynthetic enzyme is from one or more of Yersinia pestis, Pseudomonas aeruginosa, Acinetobacter baumannii, Francisella novicida, E. coli, Bordetella subspecies, Helicobacter pylori, Leptospira interrogans or Salmonella typhimurium. In some embodiments, the lipid A-modifying enzyme can include phoP, lpxP, msbB, lpxE, pagP, and/or lpxF.

Upon production of the lipooligosaccharide/lipid A-based mimetics in the selected bacterial strain, the lipooligosaccharide/lipid A-based mimetics are obtained from the bacteria. The mimetic molecules may be obtained by any suitable method, but in specific embodiments they are chemically extracted using standard LOS extraction protocols. In specific cases, initially multiple types of LOS extraction procedures are employed to obtain LOS from the bacteria, and extraction procedures may be performed more than once. In particular aspects, the extraction procedures are phenol-based, masnesium-precipitation-based, ammonium hydroxide/isobutyric acid-based, chloroform-methanol-based, detergent-based, and so forth.

Once the LOS preparation is obtained from the bacteria, the lipid A fraction is liberated using gentle hydrolysis to protect sensitive structural elements.

In particular embodiments, lipid A or its mimetics may be isolated as follows. Lipid A was isolated after hydrolysis in 1% SDS at pH 4.5. Briefly, 500 μl of 1% SDS in 10 mM Na-acetate (pH 4.5) was added to a lyophilized sample. Samples were incubated at 100° C. for 1 h, frozen, and lyophilized. The dried pellets were resuspended in 100 μl of water and 1 ml of acidified ethanol (100 μl 4 N HCl in 20 ml 95% ethanol). Samples were centrifuged at 5,000 rpm for 5 min. The lipid A pellet was further washed (three times) in 1 ml of 95% ethanol. The entire series of washes was repeated twice. Samples were resuspended in 500 μl of water, frozen on dry ice, and lyophilized. Lipid A was used for matrix-assisted laser desorption ionization (MALDI) mass spectrometry analysis.

The lipid A mimetic is most desirably administered at a concentration level that will generally afford adjuvant activity without causing any harmful or deleterious side effects. Generally, an effective adjuvant amount is desired. An effective adjuvant amount refers to an amount of an adjuvant which is capable of stimulating an immune response to an administered immunogen. In some embodiments, the lipid A mimetic is administered at a dose of between about 0.1-500 ug, between about 1.0-250 ug, between about 5.0-150 ug, between about 10-100 ug, between about 25-75 ug, or between about 35-50 ug. In some embodiments, the lipid A mimetic is administered at a dose of about 50 ug.

An “immunogen” is an entity that induces an immune response in a subject, i.e., induces an innate or an adaptive host response. In some embodiments, the response is capable of protecting the host from infection. As used herein, the term “immunogen” can include an antigen. The immunogen can also include e.g., a split virus, a subunit antigen, an inactivated whole virus/whole virion, an attenuated virus, and combinations thereof. An inactivated whole virus can be chemically inactivated by any suitable means including, for example, by treating with formaldehyde, formalin, or β-propiolactone, or otherwise inactivated such as by ultraviolet or heat inactivation. Additional chemical means for inactivation include treatment with methylene blue, psoralen, carboxyfullerene (C60) or a combination thereof. Other methods of viral inactivation are known in the art, such as for example binary ethylamine, acetyl ethyleneimine, or gamma irradiation. The immunogen can be provided in a purified or an unpurified form. For example, a purification process may involve zonal centrifugation using a linear sucrose gradient solution that includes detergent to disrupt the virions. Antigens may then be purified, after optional dilution, by diafiltration.

In some embodiments, the immunogen is an antigen. As used herein, “antigen” refers to a biomolecule capable of eliciting an immune response in a host. In some embodiments, an antigen may be a protein, or fragment of a protein. Another type of antigen in which a lipid A mimetic is used as an adjuvant includes genetic antigens, wherein an immune response may be promoted by transfecting or inoculating an animal with a nucleic acid encoding an antigen, following which one or more cells comprised within a target animal then expresses the sequences encoded by the nucleic acid after administration of the nucleic acid to the animal; the antigen may also be in the form, for example, of a nucleic acid (e.g., a cDNA or an RNA) encoding all or part of the peptide or polypeptide sequence of an antigen. Expression in vivo by the nucleic acid may be, for example, by a plasmid type vector, a viral vector, a viral/plasmid construct vector, or nanoparticles, e.g., solid lipid nanoparticles. In yet another case, a lipid A mimetic is employed with a cellular antigen comprising a cell expressing the antigen, such as a cell isolated from a culture, tissue, organ or organism. The cell may be transfected with a nucleic acid encoding an antigen to enhance its expression of the antigen. Of course, the cell may also express one or more additional components, such as immunomodulators or adjuvants (other than the lipid A mimetic adjuvant). An immunogenic composition may comprise all or part of the cell.

In an exemplary embodiment, the antigen elicits a protective immune response. As used herein, “protective” means that the immune response contributes to the lessening of any symptoms associated with infection of a host with the pathogen the antigen was derived from or designed to elicit a response against. For example, a protective antigen from a pathogen may induce an immune response that helps to ameliorate symptoms associated with the infection by the pathogen or reduce the morbidity and mortality associated with infection with the pathogen. The use of the term “protective” in this invention does not necessarily require that the host is completely protected from the effects of the pathogen.

In some embodiments, the influenza or other virus immunogen can be derived from the conventional embryonated egg method, or can be derived from any methods using tissue culture to grow the virus or express recombinant influenza virus surface antigens. Suitable cell substrates for growing the virus include, for example, dog kidney cells such as Madin-Darby Canine Kidney (MDCK) or cells from a clone of MDCK, MDCK-like cells, monkey kidney cells such as African Green Monkey kidney (AGMK) cells including Vero cells, suitable pig cell lines, or any other mammalian cell type suitable for the production of influenza or other viruses for vaccine purposes. Suitable cell substrates also include human cells, e.g., Medical Research Council strain 5 (MRC-5) cells. Suitable cell substrates are not limited to cell lines; for example primary cells such as chicken embryo fibroblasts are also included.

In some embodiments, the influenza or other virus immunogen can be attenuated, temperature-sensitive, and/or cold-adapted.

The virus immunogen can be produced by any of a number of commercially applicable processes, for example a split flu process. Traditionally split flu is produced using a solvent/detergent treatment, such as tri-n-butyl phosphate, or diethylether in combination with Tween® (known as “Tween-ether” splitting). Other splitting agents now employed include detergents or proteolytic enzymes or bile salts, for example sodium deoxycholate. Detergents that can be used as splitting agents include cationic detergents, e.g., cetyl trimethyl ammonium bromide (CTAB), other ionic detergents, e.g., laurylsulfate, taurodeoxycholate, or non-ionic detergents including Triton™ X-100 and Triton™ N-101, or combinations of any two or more detergents. Additional splitting agents can include alkylglycosides, alkylthioglycosides, acyl sugars, sulphobetaines, betains, polyoxyethylenealkylethers, N,N-dialkyl-glucamides, Hecameg®, alkylphenoxy-polyethoxyethanols, quaternary ammonium compounds, sarcosyl, CTABs (cetyl trimethyl ammonium bromides), tri-N-butyl phosphate, Cetavlon, myristyltrimethylammonium salts, lipofectin, lipofectamine, and DOT-MA, the octyl- or nonylphenoxy polyoxyethanols (e.g. the Triton surfactants mentioned above), polyoxyethylene sorbitan esters (the Tween® surfactants), polyoxyethylene ethers, polyoxyethlene esters, etc. The preparation process for a split vaccine can include a number of different filtration and/or other separation steps such as ultracentrifugation, ultrafiltration, zonal centrifugation and chromatography (e.g., ion exchange) steps in a variety of combinations, and optionally an inactivation step, e.g., with heat, formaldehyde or β-propiolactone or ultraviolet which can be carried out before or after splitting. The splitting process can be carried out as a batch, continuous or semi-continuous process.

In some embodiments, the immunogen may be derived from a source other than the live virus. For example, for influenza, a hemagglutinin, matrix, neuraminidase, or nucleoprotein antigen can be produced recombinantly in a recombinant host, e.g., an insect cell line using a baculovirus vector, and used in purified form.

In some embodiments, the antigen is a polypeptide. In some embodiments, the antigen is a fragment or variant sequence of a wild-type sequence. The antigenic fragment can be “free-standing,” or comprised within a larger polypeptide of which they form a part or region, most preferably as a single continuous region.

In some embodiments, the antigenic fragment can include, for example, truncation polypeptides having the amino acid sequence of a polypeptide, except for deletion of a continuous series of residues that includes the amino terminus, or a continuous series of residues that includes the carboxyl terminus or deletion of two continuous series of residues, one including the amino terminus and one including the carboxyl terminus. Discontinuous fragments are also envisaged, e.g., a full length protein antigen having one or more internal deletions. In some embodiments, fragments are characterized by structural or functional attributes such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, and high antigenic index regions.

The fragment can be of any size. An antigenic fragment is capable of inducing an immune response in a subject or be recognized by a specific antibody. In some embodiments, the fragment corresponds to an amino-terminal truncation mutant. In some embodiments, the number of amino terminal amino acids missing from the fragment ranges from 1-100 amino acids. In some embodiments, it ranges from 1-75 amino acids, 1-50 amino acids, 1-40 amino acids, 1-30 amino acids, 1-25 amino acids, 1-20 amino acids, 1-15 amino acids, 1-10 amino acids and 1-5 amino acids.

In some embodiments, the fragment corresponds to carboxyl-terminal truncation mutant. In some embodiments, the number of carboxyl terminal amino acids missing from the fragment ranges from 1-100 amino acids. In some embodiments, it ranges from 1-75 amino acids, 1-50 amino acids, 1-40 amino acids, 1-30 amino acids, 1-25 amino acids, 1-20 amino acids, 1-15 amino acids, 1-10 amino acids and 1-5 amino acids.

In some embodiments, the fragment corresponds to an internal fragment that lacks both the amino and carboxyl terminal amino acids. In some embodiments, the fragment is 7-200 amino acid residues in length. In some embodiments, the fragment is 10-100 amino acid residues, 15-85 amino acid residues, 25-65 amino acid residues or 30-50 amino acid residues in length. In some embodiments, the fragment is 7 amino acids, 10 amino acids, 12 amino acids, 15 amino acids, 20 amino acids, 25 amino acids, 30 amino acids, 35 amino acids, 40 amino acids, 45 amino acids, 50 amino acids 55 amino acids, 60 amino acids, 80 amino acids or 100 amino acids in length.

In some embodiments, the fragment is a discontinuous fragment that lacks one or more regions of the protein. In some embodiments, the fragment is 30-400 amino acid residues in length. In some embodiments, the fragment is 30-300 amino acid residues, 30-250 amino acid residues, 30-200 amino acid residues or 30-100 amino acid residues in length.

In some embodiments, the fragment is at least 50 amino acids, 100 amino acids, 150 amino acids, 200 amino acids or at least 250 amino acids in length. Of course, larger antigenic fragments are also useful according to the present invention, as are fragments corresponding to most, if not all, of the amino acid sequence of a polypeptide from which it is derived.

In some embodiments, the antigenic polypeptide has an amino acid sequence at least 80, 85%, 90%, 91%, 92%, 93%, 94%, 95%, 96%, 97%, 98%, 99%, or 100% identical to the polypeptide antigens described herein or antigenic or biologically active fragments thereof. In some embodiments, the variants are those that vary from the reference by conservative amino acid substitutions, i.e., those that substitute a residue with another of like characteristics. Typical substitutions are among Ala, Val, Leu and Ile; among Ser and Thr; among the acidic residues Asp and Glu; among Asn and Gln; and among the basic residues Lys and Arg, or aromatic residues Phe and Tyr. In some embodiments, the polypeptides are variants in which several, 5 to 10, 1 to 5, or 1 to 2 amino acids are substituted, deleted, or added in any combination.

In some embodiments, the polypeptides are encoded by polynucleotides that are optimized for high level expression in a microorganism, such as E. coli, using codons that are preferred in the microorganism. As used herein, a codon that is “optimized for high level expression refers to a codon that is relatively more abundant in the microorganism in comparison with all other codons corresponding to the same amino acid. In some embodiments, at least 10% of the codons are optimized for high level expression. In some embodiments, at least 25%, at least 50%, at least 75%, at least 80%, at least 85%, at least 90%, at least 95%, or at least 99% of the codons are optimized for high level expression.

The immunogen to be formulated into a vaccine of the present invention is not particularly limiting and can include, e.g., one or more antigens from a virus, a split virus, an inactivated whole virus/whole virion, an attenuated virus, and combinations thereof. The virus can include, but is not limited to Influenza A strains, seasonal and non-seasonal, including H1N1 (“Spanish flu”/“Swine flu”), H2N2 (“Asian flu”), H3N2 (“Hong Kong flu”), H5N1 (“Avian flu”/“Bird flu”), H7N7, H1N2, H9N2, H7N2, H7N3 and H10N7, Influenza B, Influenza C, Influenza D, coronaviruses such as severe acute respiratory syndrome coronavirus (SARS-CoV), SARS-CoV-2 (causes COVID-19), other SARS viruses, Middle East respiratory syndrome-related coronavirus (MERS-CoV), human coronaviruses including HCoV-0C43, HCoV-HKU1, HCoV-229E and HCoV-NL63, rhinoviruses of the Enterovirus genus such as human rhinovirus 2 (HRV2), HRV3, HRV14, HRV16 and HRV29, respiratory syncytial virus (RSV), human papillomaviruses including but not limited to HPV-1, HPV-2, HPV-3, HPV-4, HPV-6, etc., and combinations thereof. In some embodiments, the antigen is presented in the context of a synthetic viral like particle (VLP).

In some embodiments, the immunogen to be formulated into a pharmaceutical composition of the present invention can be derived from poxviruses, e.g., smallpox virus, cowpox virus and orf virus; herpes viruses, e.g., herpes simplex virus type 1 and 2, B-virus, varicella zoster virus, cytomegalovirus, and Epstein-Barr virus; adenoviruses, e.g., mastadenovirus; papovaviruses, e.g., papillomaviruses such as HPV16, and polyomaviruses such as BK and JC virus; parvoviruses, e.g., adeno-associated virus; reoviruses, e.g., reoviruses 1, 2 and 3; orbiviruses, e.g., Colorado tick fever; rotaviruses, e.g., human rotaviruses; alphaviruses, e.g., Eastern encephalitis virus and Venezuelan encephalitis virus; rubiviruses, e.g., rubella; flaviviruses, e.g., yellow fever virus, Dengue fever viruses, Japanese encephalitis virus, Tick-borne encephalitis virus and hepatitis C virus; coronaviruses, e.g., human coronaviruses; paramyxoviruses, e.g., parainfluenza 1, 2, 3 and 4 and mumps; morbilliviruses, e.g., measles virus; pneumovirus; vesiculoviruses, e.g., vesicular stomatitis virus; lyssaviruses, e.g., rabies virus; orthomyxoviruses; bunyaviruses e.g., LaCrosse virus; phleboviruses, e.g., Rift Valley fever virus; nairoviruses, e.g., Congo hemorrhagic fever virus; hepadnaviridae, e.g., hepatitis B; arenaviruses, e.g., 1 cm virus, Lasso virus and Junin virus; retroviruses, e.g., HTLV I, HTLV II, HIV-1 and HIV-2; enteroviruses, e.g., polio virus 1,-2 and 3, coxsackie viruses, echoviruses, human enteroviruses, hepatitis A virus, hepatitis E virus, and Norwalk-virus; rhinoviruses e.g., human rhinovirus; and filoviridae, e.g., Marburg (disease) virus, Ebola virus, combinations thereof and combinations with the viruses of the preceding paragraph.

In some embodiments, depending on the season and on the nature of the antigen included, compositions of the invention can potentially protect against one or more of Influenza A virus hemagglutinin subtypes H1, H2, H3, H4, H5, H6, H7, H8, H9, H10, H11, H12, H13, H14, H15 or H16. In some embodiments, the invention can potentially protect against one or more of Influenza A virus NA subtypes N1, N2, N3, N4, N5, N6, N7, N8 or N9.

In some embodiments, antigens from more than one virus strains can be mixed in the compositions of the present invention. In some embodiments, the pharmaceutical compositions according to the present invention can be monovalent, divalent, trivalent, or otherwise multivalent.

In some embodiments, the immunogen comprises hemagglutinin (HA) protein or an antigenic fragment or variant thereof from an Influenza A or B strain. In some embodiments, the HA protein is a purified, recombinant HA. In some embodiments, the strain is the A/California/04/2009 (pandemic H1N1) (Cal/09) virus. The recombinant protein can be expressed in a number of ways and is not limiting. In some embodiments, the antigen can be expressed from baculovirus vectors in High Five cells.[22] The Cal/09 rHA is phylogenetically matched to the A/Netherlands/602/2009 (NL/09) virus used in all of the homologous infections as shown in the Examples below. In some embodiments, there are advantages of using rHA over split virion grown in eggs during vaccine production: this strategy is not dependent on the availability of eggs, eliminates the need to find virus adapted for growth in eggs, and importantly, avoids allergic reactions to eggs in vaccinees. In some aspects, another major advantage in using rHA antigen for an influenza vaccine is production time. For rapid response, rHA has a shorter manufacture timeframe than do other influenza immunogens, and this is valuable in response to a pandemic outbreak.[23, 24]

In some embodiments, the viral immunogen is from a respiratory syncytial virus (RSV). In some embodiments, the RSV immunogen is an RSV F protein or an antigenic fragment or variant thereof. In some embodiments, the immunogen is a stabilized prefusion F0 protein. See, e.g., McLellan et al., Science. 2013 May 31; 340(6136): 1113-1117, which is incorporated by reference herein. A stabilized prefusion F0 protein can be a soluble form of the RSV F protein in its pre-fusion state that created by introducing a disulfide bond, two cavity-filling mutations, and a C-terminal Foldon trimerization domain (as described in McLellan et al., supra). This pre-fusion stabilized version is unable to mature into the fusion competent version and, therefore, maintains all endogenous epitopes as seen on circulating RSV-A2.

In some embodiments, the viral immunogen is from a human papillomavirus (HPV). In some embodiments, the HPV immunogen is L1 or a fragment or a variant thereof and/or L2, or a fragment or variant thereof. In some embodiments, the immunogen comprises the RG1 sequence. In some embodiments, the immunogen is a virus like particle comprising L1 and/or L2 or fragments or variants thereof. In some embodiments, the immunogen is RG1-VLP. See, e.g., Schellenbacher et al. J Invest Dermatol. 133:2706 (2013). In some embodiments, the RG1-VLP that is useful is as described below in the Examples.

As used herein, the term “subject” includes both human and animal subjects. The term “subject” includes a human or other animal at risk of developing or suffering from viral infection, in particular a respiratory infection such as influenza. Veterinary therapeutic uses are also provided. Examples of non-human mammals include, but are not limited to, cats, dogs, swine, including pigs, hogs, and wild boars, ruminants and/or ungulates such as cattle, oxen, sheep, giraffes, deer, goats, bison, and camels, and horses. Also provided is the treatment of birds as well as fowl, and more particularly domesticated fowl, i.e., poultry, such as turkeys, chickens, ducks, geese, guinea fowl, and the like. In some embodiments, the subject is a mammal. In some embodiments, the subject is a human. In some embodiments, the viral immunogen is from an influenza A and/or B virus.

In some embodiments, the subject is a human 55 years old or greater, the adjuvant is BECC470 and the immunogen, e.g., rHA, is for influenza.

Vaccine strategies are well known in the art and therefore the vaccination strategy encompassed by the invention does not limit the invention in any manner. In certain aspects of the invention, the pharmaceutical composition is administered alone in a single application or administered in sequential applications, spaced out over time.

As used herein, an “immune response” is the physiological response of the subject's immune system to an immunizing composition. An immune response may include an innate immune response, an adaptive immune response, or both. In one embodiment of the present invention, the immune response is a protective immune response. A protective immune response confers immunological cellular memory upon the subject, with the effect that a secondary exposure to the same or a similar antigen is characterized by one or more of the following characteristics: shorter lag phase than the lag phase resulting from exposure to the selected antigen in the absence of prior exposure to the immunizing composition; production of antibody which continues for a longer period than production of antibody resulting from exposure to the selected antigen in the absence of prior exposure to the immunizing composition; a change in the type and quality of antibody produced in comparison to the type and quality of antibody produced upon exposure to the selected antigen in the absence of prior exposure to the immunizing composition; a shift in class response, with IgG antibodies appearing in higher concentrations and with greater persistence than IgM, than occurs in response to exposure to the selected antigen in the absence of prior exposure to the immunizing composition; an increased average affinity (binding constant) of the antibodies for the antigen in comparison with the average affinity of antibodies for the antigen resulting from exposure to the selected antigen in the absence of prior exposure to the immunizing composition; and/or other characteristics known in the art to characterize a secondary immune response.

In some embodiments, the immune response is sufficient to confer protective immunity upon the subject against a later infection by the viral pathogen.

In other aspects of the invention, the pharmaceutical composition is administered as a component of a prime/boost regimen. The prime/boost regimen can be classified as “heterologous prime/boost” or “homologous prime/boost.” Heterologous prime/boost strategies are 2-phase immunization regimes involving sequential administration (in a priming phase and a boosting phase) of the same antigen in two different vaccine formulations by the same or different route. In particular aspects of the invention drawn to homologous prime/boost regimens, a parenteral prime/parenteral boost immunization strategy is used. In some embodiments drawn to homologous prime/boost regimens, a mucosal prime/mucosal boost immunization strategy is used. In some embodiments of the invention drawn to heterologous prime/boost regimens, a mucosal prime is followed by a parenteral boost immunization. In some embodiments of the invention drawn to heterologous prime/boost regimens, a parenteral prime is followed by a mucosal boost immunization. Any combination of prime and boost are possible, and the subject can be primed and/or boosted 1, 2, 3, 4, 5, 6 or more times. In some embodiments, the composition is administered intradermally, e.g., into the epidermal or dermal layers of the skin. In some embodiments, the composition is administered intranasally. In some embodiments, the pharmaceutical composition is administered by intramuscular injection.

In some embodiments, a composition comprising the immunogen is administered, e.g., mucosally in a first priming administration, followed, optionally, by a second (or third, fourth, fifth, etc. . . . ) priming administration of the composition from about 2 to about 10 weeks later. In some embodiments, a boosting composition is administered from about 3 to about 12 weeks after the priming administration. In some embodiments, the boosting composition is administered from about 3 to about 6 weeks after the priming administration. In some embodiments, the boosting composition is substantially the same type of composition administered as the priming composition (e.g., a homologous prime/boost regimen).

The manner of administration of the inventive vaccines may be varied widely. Any of the conventional methods for administration are applicable. For example, the composition may be conventionally administered intravenously, intradermally, intraarterially, intraperitoneally, intralesionally, intracranially, intraarticularly, intraprostaticaly, intrapleurally, intratracheally, intranasally, intravitreally, intravaginally, intratumorally, intramuscularly, intraperitoneally, subcutaneously, intravesicularlly, mucosally, intrapericardially, orally, rectally, nasally, topically, in eye drops, locally, using aerosol, injection, infusion, continuous infusion, localized perfusion, via a catheter, via a lavage, in creams, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

In practicing immunization protocols for treatment and/or prevention, an immunologically-effective amount of the immunogen or composition comprising the immunogen is administered to a subject. As used herein, the term “immunologically-effective amount” means the total amount that is sufficient to show an enhanced immune response in the subject. When “immunologically-effective amount” is applied to an individual therapeutic agent administered alone, the term refers to that therapeutic agent alone. When applied to a combination, the term refers to combined amounts of the ingredients that result in the therapeutic effect, whether administered in combination, serially or simultaneously. In some embodiments, an effective amount refers to an amount that is of sufficient quality and quantity to neutralize, ameliorate, modulate, or reduce the cause of or effect of a viral infection in a subject by stimulating an immune response in the subject. By “ameliorate,” “modulate,” or “reduce” is meant a lessening or reduction or prophylactic prevention of the detrimental effect of the viral infection in the subject receiving the vaccine, thereby resulting in “protecting” the subject. A “sufficient amount” or “effective amount” or “therapeutically effective amount” of an administered composition is that volume or concentration which causes or produces a measurable change from the pre-administration state in the cell or patient. The subject of the invention can be a human subject, however, it can be envisioned that any animal at risk for viral infection, such as a mouse, can be treated in a method of the present invention.

Of course, the effective amount in any particular case will depend upon a variety of factors including the activity of the therapeutic vaccine composition, formulation, the route of administration, combination with other drugs or treatments, and the physical condition and prior medical history of the subject being treated. Determination and adjustment of a therapeutically effective dose of a vaccine are known to those of ordinary skill in the art.

A dosing schedule may be varied on a patient by patient basis, taking into account, for example, factors such as the weight and age of the patient, the viral disease being targeted, previous or concurrent therapeutic interventions, the manner of administration and the like, which can be readily determined by one of ordinary skill in the art. In some embodiments, the vaccine is administered in a dose that provides the immunogen in an amount of from about 50 ng/kg to about 1.0 mg/kg body weight. In some embodiments, the immunogen delivered is about 100 ng to about 500 μg/kg body weight of the subject. In some embodiments, the dose administered provides the immunogen in an amount of about 1-100 μg/kg body weight of the subject. In some embodiments, the dose administered provides the immunogen in an amount of about 50 μg/kg body weight of the subject. In some embodiments, the vaccine is administered in a dose that provides the adjuvant in an amount of from about 15 μg/kg to about 1.9 mg/kg body weight. In some embodiments, the adjuvant delivered is about 100 μg to about 1000 μg/kg body weight of the subject. In some embodiments, the dose administered provides the adjuvant in an amount of about 200-500 μg/kg body weight of the subject. In some embodiments, the dose administered provides the adjuvant in an amount of about 400 μg/kg body weight of the subject.

In some embodiments, the immunologically-effective amount of the viral immunogen administered is from about 50 ng to about 1.0 mg per kg of body weight of the subject. In some embodiments, the effective amount of the viral immunogen administered is from about 15 μg to about 1.9 mg per kg of body weight of the subject.

In some embodiments, an appropriate concentration of immunogen for each virus strain included in a composition of the present invention is an amount which induces an immune response without significant, adverse side effects.

Specific dosages may be adjusted depending on conditions of disease, the age, body weight, general health conditions, sex, diet of the subject, dose intervals, administration routes, excretion rate, and combinations of drugs. Any of the dosage forms described herein containing effective amounts are well within the bounds of routine experimentation and therefore, well within the scope of the instant disclosure.

The desired dose may be presented in a single dose or as divided doses administered at appropriate intervals, for example as two, three, four or more sub-doses per day. Optionally, a dose of vaccine may be administered on one day, followed by one or more booster doses spaced as desired thereinafter, although in some embodiments, booster doses are not required for the vaccine to protect against infection. In one exemplary embodiment, an initial vaccination is given, followed by a boost of the same vaccine approximately one week to 15 days later.

In some embodiments, administration of the compositions herein will induce immunity quickly and with a single administration; however, in the absence of a live replicating pathogen, this is not always possible. Ways to increase immunity with adjuvanted protein-based vaccines include, in addition to using prime-boost or prime-boost-boost strategies, alternative strategies in which the vaccine recipient is dosed multiple times with the formulated antigenic protein to mimic repeated exposure, leading to a protective immune response. Specifically, proposed vaccine combinations can be evaluated to determine whether a single prime immunization or prime-boost is sufficient to provide the desired 100% protective immunity in the influenza virus models. Ultimately, boosting the antigen immunogenicity through the use of a more effective adjuvant represents a significant advancement in the development of influenza vaccines.

The multiple doses of a particular vaccine administration strategy can typically be administered at least 1 week apart (e.g. at least about 2 weeks, about 3 weeks, about 4 weeks, about 6 weeks, about 8 weeks, about 10 weeks apart, about 12 weeks, about 16 weeks apart, etc.).

It is contemplated that the compositions herein include the viral immunogen and the lipid A-based mimetic(s), but there may also be one or more additional components to form a more effective antigenic composition. Non-limiting examples of additional components include, for example, one or more additional immunogens, immunomodulators or adjuvants to stimulate an immune response to the antigenic composition.

For example, in some embodiments, one or more immunomodulators can be included in the composition to augment a cell's or a patient's (e.g., an animal's) response. Immunomodulators can be included as purified proteins, nucleic acids encoding immunomodulators, and/or cells that express immunomodulators in the vaccine composition. In specific embodiments, various combinations of immunomodulators may be used (e.g., a cytokine and a chemokine). When cytokines are included in the compositions, they may be interleukins, cytokines, nucleic acids encoding interleukins or cytokines, and/or cells expressing such compounds are contemplated as possible vaccine components. Interleukins and cytokines include but are not limited to interleukin 1 (IL-1), IL-2, IL-3, IL-4, IL-5, IL-6, IL-7, IL-8, IL-9, IL-10, IL-11, IL-12, IL-13, IL-14, IL-15, IL-18, IL-22, IL-23 β-interferon, α-interferon, g-interferon, angiostatin, thrombospondin, endostatin, GM-CSF, G-CSF, M-CSF, METH-1, METH-2, tumor necrosis factor, TGFβ, LT and combinations thereof. In some cases, chemokines, nucleic acids that encode for chemokines, and/or cells that express such also may be used as components of the composition. Chemokines generally act as chemoattractants to recruit immune effector cells to the site of chemokine expression. It may be advantageous to express a particular chemokine coding sequence in combination with, for example, a cytokine coding sequence, to enhance the recruitment of other immune system components to the site of treatment. Such chemokines include, for example, RANTES, MCAF, MIP1-alpha, MIP1-Beta, IP-10 and combinations thereof. The skilled artisan will recognize that certain cytokines are also known to have chemoattractant effects and could also be classified under the term chemokines. Other examples of molecules to include in the composition are immunogenic carrier proteins, such as hepatitis B surface antigen, keyhole limpet hemocyanin (KLH), bovine serum albumin (BSA), ovalbumin, mouse serum albumin or rabbit serum albumin. Biological response modifiers (BRM) may be utilized that have been shown to upregulate T cell immunity or downregulate suppressor cell activity. Such BRMs include, but are not limited to, cimetidine (CIM; 1200 mg/d) (Smith/Kline, PA); low-dose cyclophosphamide (CYP; 300 mg/m²) (Johnson/Mead, NJ), or a gene encoding a protein involved in one or more immune helper functions, such as B-7.

Various methods known to those skilled in the art can be used to determine a reduction in the amount of a marker or symptom associated with viral disease, or other disease or disorder, in a subject. For example, in some embodiments, the amounts of expression of an inflammatory marker in a subject can be determined by probing for mRNA of the gene encoding the inflammatory marker in a biological sample obtained from the subject (e.g., a tissue sample, a urine sample, a saliva sample, a blood sample, a serum sample, a plasma sample, or sub-fractions thereof) using any RNA identification assay known to those skilled in the art. Briefly, RNA can be extracted from the sample, amplified, converted to cDNA, labeled, and allowed to hybridize with probes of a known sequence, such as known RNA hybridization probes immobilized on a substrate, e.g., array, or microarray, or quantitated by real time PCR (e.g., quantitative real-time PCR, such as available from Bio-Rad Laboratories, Hercules, Calif.). Because the probes to which the nucleic acid molecules of the sample are bound are known, the molecules in the sample can be identified. In this regard, DNA probes for one or more of the mRNAs encoded by the inflammatory genes can be immobilized on a substrate and provided for use in practicing a method in accordance with the presently-disclosed subject matter.

In some embodiments, mass spectrometry and/or immunoassay devices and methods can also be used to measure the inflammatory cytokines in samples, although other methods can also be used and are well known to those skilled in the art. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety. Immunoassay devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, can be employed to determine the presence or amount of analytes without the need for a labeled molecule. See, e.g., U.S. Pat. Nos. 5,631,171; and 5,955,377, each of which is hereby incorporated by reference in its entirety.

Any suitable immunoassay can be utilized, for example, enzyme-linked immunoassays (ELISA), radioimmunoassays (RIAs), competitive binding assays, and the like. Specific immunological binding of the antibody to the inflammatory molecule can be detected directly or indirectly. Direct labels include fluorescent or luminescent tags, metals, dyes, radionucleotides, and the like, attached to the antibody. Indirect labels include various enzymes well known in the art, such as alkaline phosphatase, horseradish peroxidase and the like.

The use of immobilized antibodies or fragments thereof specific for the inflammatory molecules is also contemplated by the present invention for the purpose of evaluating the efficacy of a candidate vaccine. The antibodies can be immobilized onto a variety of solid supports, such as magnetic or chromatographic matrix particles, the surface of an assay plate (such as microtiter wells), pieces of a solid substrate material (such as plastic, nylon, paper), and the like. An assay strip can be prepared by coating the antibody or a plurality of antibodies in an array on a solid support. This strip can then be dipped into the test biological sample and then processed quickly through washes and detection steps to generate a measurable signal, such as for example a colored spot.

Mass spectrometry (MS) analysis can be used, either alone or in combination with other methods (e.g., immunoassays), to determine the presence and/or quantity of an inflammatory molecule in a subject. Exemplary MS analyses that can be used in accordance with the present invention include, but are not limited to: liquid chromatography-mass spectrometry (LC-MS); matrix-assisted laser desorption/ionization time-of-flight MS analysis (MALDI-TOF-MS), such as for example direct-spot MALDI-TOF or liquid chromatography MALDI-TOF mass spectrometry analysis; electrospray ionization MS (ESI-MS), such as for example liquid chromatography (LC) ESI-MS; and surface enhanced laser desorption/ionization time-of-flight mass spectrometry analysis (SELDI-TOF-MS). Each of these types of MS analysis can be accomplished using commercially-available spectrometers, such as, for example, triple quadropole mass spectrometers. Methods for utilizing MS analysis to detect the presence and quantity of peptides, such as inflammatory cytokines, in biological samples are known in the art. See, e.g., U.S. Pat. Nos. 6,925,389; 6,989,100; and 6,890,763 for further guidance, each of which are incorporated herein by this reference.

In some embodiments, a qualitative assessment is performed, e.g., detecting the presence or absence of the expression of an inflammatory marker in a subject. In some embodiments, a quantitative assessment is performed, e.g., determining an amount of decrease in the level of an inflammatory marker in a subject. Such quantitative assessments can be made, for example, using one of the above-mentioned methods, as will be understood by those skilled in the art.

In some embodiments, measuring a reduction in the amount of a certain feature (e.g., cytokine levels) or an improvement in a certain feature (e.g., inflammation) in a subject can be performed by a statistical analysis. For example, a reduction in an amount of inflammatory markers in a subject can be compared to control levels of inflammatory markers, and an amount of inflammatory markers of less than or equal to the control level can be indicative of a reduction in the amount of inflammatory markers, as evidenced by a level of statistical significance. Statistical significance is often determined by comparing two or more populations, and determining a confidence interval and/or a p value. See, e.g., Dowdy and Wearden, Statistics for Research, John Wiley & Sons, New York, 1983, incorporated herein by reference in its entirety. In some embodiments, confidence intervals are 90%, 95%, 97.5%, 98%, 99%, 99.5%, 99.9% and 99.99%, while in some embodiments, p values are selected from 0.1, 0.05, 0.025, 0.02, 0.01, 0.005, 0.001, and 0.0001.

The course of the immunization may be followed by assays for antibodies for the supernatant antigens. The assays may be performed by labeling with conventional labels, such as radionuclides, enzymes, fluorescents, and the like. These techniques are well known and may be found in a wide variety of patents, such as U.S. Pat. Nos. 3,791,932; 4,174,384 and 3,949,064, as illustrative of these types of assays. Other immune assays can be performed and assays of protection from challenge with the antigen can be performed, following immunization.

In another embodiment, the invention comprises a pharmaceutical composition of any of the compositions or a combination thereof in combination with a pharmaceutically acceptable carrier.

In some embodiments, the pharmaceutical compositions of the present invention comprise an immunogen and a lipid A mimetic adjuvant herein and can be used for prophylactic treatment which can include preventing a condition such as infection with influenza A in a subject mammal. The degree of prevention achieved can be total or partial. In some embodiments, the treatment includes inhibiting the development of viral infection. In some embodiments, the composition reduces the severity of viral infection that develops in the subject.

In some embodiments, the methods of the invention reduce risk of viral infection by at least 20%, more preferably by at least 50%, even more preferably by 80% or greater, and also, in some embodiments, in a dose-dependent manner.

In some embodiments, the lipid A mimetic adjuvant is administered in a vaccine in combination with one or more additional adjuvants for the prevention of viral infection.

The BECC adjuvants can be formulated with an immunogen alone or in combination with each other or with adjuvants known in the art to form the vaccines of the present invention. Known adjuvants include but are not limited to mineral-containing compositions, including calcium salts and Aluminum salts (or mixtures thereof). Adjuvant calcium salts can include calcium phosphate; Aluminum salts can include hydroxides, phosphates, sulfates, etc., with the salts taking any suitable form (e.g. gel, crystalline, amorphous, etc.). The mineral containing compositions may also be formulated as a particle of metal salt. Other adjuvants can include oil-in-water emulsions, immunostimulatory oligonucleotides, 3-O-deacylated monophosphoryl lipid A (“3dMPL,” also known as MPL™) and various organic heterocyclic compounds including thiosemicarbazones, imidazoquinoline compounds, such as Imiquimod (“R-837”), Resiquimod (“R-848”) and their analogs and salts thereof (e.g. the hydrochloride salts), isatorabine, acylpiperazine compounds, indoledione compounds, tetrahydraisoquinoline (THIQ) compounds, benzocyclodione compounds, aminoazavinyl compounds, aminobenzimidazole quinolinone (ABIQ) compounds, hydrapthalamide compounds, benzophenone compounds, isoxazole compounds, sterol compounds, quinazilinone compounds, pyrrole compounds, anthraquinone compounds, quinoxaline compounds, triazine compounds, pyrazalopyrimidine compounds, and benzazole compounds. A more complete list is given in U.S. Pat. No. 8,808,686 B2, which is hereby incorporated by reference in its entirety.

In some embodiments, the methods herein reduce lethality of a secondary bacterial infection in the subject administered. In some embodiments, the secondary infection is caused by Streptococcus, such as S. pneumoniae. In some embodiments, an antibiotic can be administered concurrently with the inventive compositions. In some embodiments, the antibiotic is selected from Amikacin, Gentamicin, Kanamycin, Neomycin, Netilmicin, Tobramycin, Paromycin, Streptomycin, Spectinomycin, Rifaximin, Ertapenem, Doripenem, Cilastatin, Meropenen, Cefadroxil, Cefazolin, Cefalexin, Cefaclor, Cefamandole, Cefoxitin, Cefoperazone, Cefotaxime, Ceftazidime, Ceftibuten, Cefepime, Ceftaroline fosamil, Ceftibiprole, Teicoplanin, Vancomycin, Telavancin, Dalbavancin, Clindamycin, Lincomycin, Daptomycin, Azithromycin, Clarithtomycin, Erythromycin, Roxithromycin, Telithromycin, Aztreonam, Furazolidone, Penicillin, Amoxicillin, Ampicillin, Piperacillin, Tazobactam, Tiracillin, Bacitracin, Colistin, Polymixin B, Ciprofloxacin, Enoxacin, Vancocin, Claforan, Ceftriaxone, Gentamicin, Gatifloxacin, Gemifloxacin, Lefofloxacin, Nalidixic acid, Norfloxacin, Ofloxacin, Mafenide, Sulfacetamide, Sulfadiazine, Sulfadimethoxine, Sulfamethoxazole, Sulfasalazine, Sulfisoxazole, Demeclocycline, Doxcycline, Mupirocin, Tigecycline, Minocycline, Oxytetracycline, Tetracycline, Clofazimine, Dapsone, Capreomycin, Cycloserine, Ethambutol, Isoniazid, Rifabutin, Thiamphenicol, Trimethoprim, Metranidzole and combinations thereof.

In some embodiments, more than one lipid A mimetic is employed in a vaccine, including two, three, or more lipid A mimetics. The lipid A mimetic can be formulated for use as a pharmaceutical composition, including having an appropriate carrier excipient. A therapeutically effective amount of the composition(s) is employed, and the proper amount may be determined by any suitable method in the art. The vaccine composition can be delivered to the individual by any appropriate means, including by injection or orally. Multiple deliveries of the inventive vaccine may be employed.

In some embodiments, the compositions of the present invention are prepared to be pharmacologically acceptable. Pharmaceutical compositions of the present invention comprise an effective amount of an immunogen and an effective amount of one or more lipid A derivative adjuvants dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The means of preparation of a pharmaceutical composition that contains an immunogen and at least one lipid A derivative adjuvant and, optionally, an additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by the FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). The compositions may comprise different types of carriers depending on whether it is to be administered in solid, liquid or aerosol form, and whether it need to be sterile for such routes of administration as injection. Except insofar as any conventional carrier is incompatible with the active ingredient, its use in the therapeutic or pharmaceutical compositions is contemplated.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The composition may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments, the composition is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assailable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

In some embodiments, the composition must be stable under the conditions of manufacture and storage, and preserved against the contaminating action of microorganisms, such as bacteria and fungi.

In particular embodiments, prolonged absorption of an injectable composition can be brought about by the use in the compositions of agents delaying absorption, such as, for example, Aluminum monostearate, gelatin or combinations thereof.

In some embodiments, it will be desirable to have multiple administrations of the composition. In some embodiments, the composition is administered 1, 2, 3, 4, 5, 6, 7, 8, 9 or at least 10 times to the subject.

Any of the compositions described herein may be comprised in a kit. In a non-limiting example, one or more lipooligosaccharide/lipid A-based mimetics and/or bacterial strains to produce them and/or reagents for modifying the bacteria are comprised in a kit. In certain aspects, one or more reagents for modifying, culturing, and/or extracting from bacteria are include in the kit.

In specific embodiments, one or more lipooligosaccharide/lipid A-based mimetics are included in the kit and may or may not be formulated with another agent. The other agent may be an immunogenic composition itself, such as a vaccine, or it may be part of an immunogenic composition, such as an antibody, a weakened microbe, a killed microbe, one or more antigens, a toxoid, polysaccharide, or nucleic acid. The lipooligosaccharide/lipid A-based mimetics may be formulated for delivery to a mammal or may be provided with one or more reagents to produce a formulation for delivery to a mammal. The bacterial strain of the kit may be provided as a bacterial stab, bacterial slant, frozen glycerol stock, or freeze dried powder, as examples. The bacteria may be provided in the kit at a particular desired temperature, including between frozen (−80° C.) and room temperature. In embodiments, the kit comprises one or more reagents for modifying a bacteria, such as reagents to handle a recombinant vector and/or reagents to assay the bacteria for presence of the vector (such as PCR reagents, restriction enzymes, polymerases, ligases, buffers, nucleotides, etc.).

The components of the kits may be packaged either in aqueous media or in lyophilized form. The container means of the kits will generally include at least one vial, test tube, flask, bottle, syringe or other container means, into which a component may be placed, and preferably, suitably aliquotted. Where there are more than one component in the kit, the kit also will generally contain a second, third or other additional container into which the additional components may be separately placed. However, various combinations of components may be comprised in a vial. The kits of the present invention also will typically include a means for containing the compositions in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. In some cases, the container means may itself be a syringe, pipette, and/or other such like apparatus, from which the formulation may be applied to a desired area of the body, injected into an animal, and/or even applied to and/or mixed with the other components of the kit. However, the components of the kit may be provided as dried powder(s). When reagents and/or components are provided as a dry powder, the powder can be reconstituted by the addition of a suitable solvent. It is envisioned that the solvent may also be provided in another container means. The kits may also comprise a container means for containing a sterile, pharmaceutically acceptable buffer and/or other diluent.

Application of the teachings of the present invention to a specific problem is within the capabilities of one having ordinary skill in the art in light of the teaching contained herein. Examples of the compositions and methods of the invention appear in the following non-limiting Examples.

### EXAMPLES

### Example 1. BECC Adjuvanted Vaccine Provides Cross-Protection from Both Homologous and Heterologous Influenza A Infections

Influenza A virus (IAV) is a leading cause of respiratory disease worldwide often resulting in hospitalization or death. IAV has a high rate of mutation allowing for new variants to evade host immune systems and creating a need for yearly changes in the seasonal vaccine. In this study TLR4 immunostimulatory molecules, BECC438 and BECC470, are found to be superior IAV vaccine adjuvants when compared to the classic adjuvant alhydrogel, and PHAD, a synthetic TLR4 agonist. BECC adjuvants allow for antigen sparing of a recombinant HA (rHA) protein, elicit a more balanced IgG1/IgG2a response, and are protective when only a single dose is administered. Importantly, BECC adjuvants afford protection from a heterologous IAV strain demonstrating that a cross-protective influenza vaccine is possible when the antigen is effectively adjuvanted.

### Results

Determination of Concentration of Unadjuvanted rHA that Confers Protection to Homologous Challenge

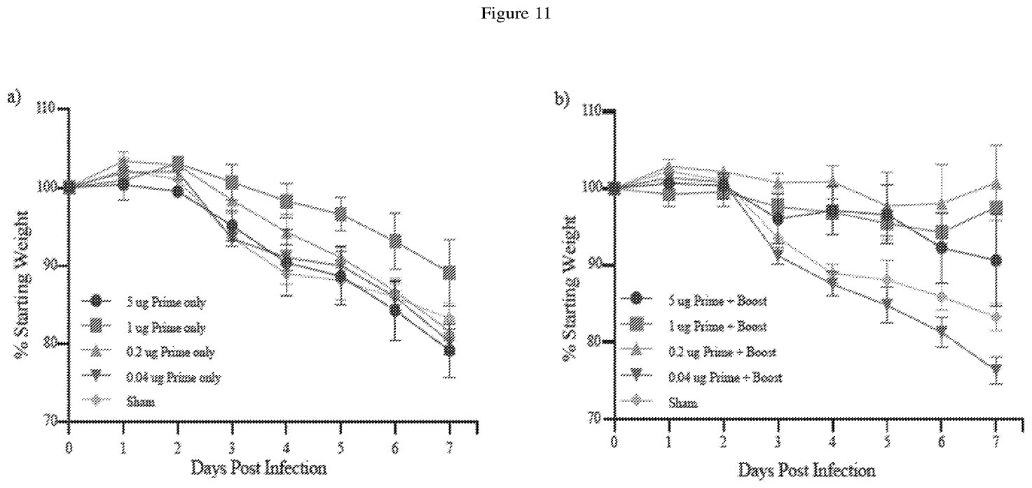

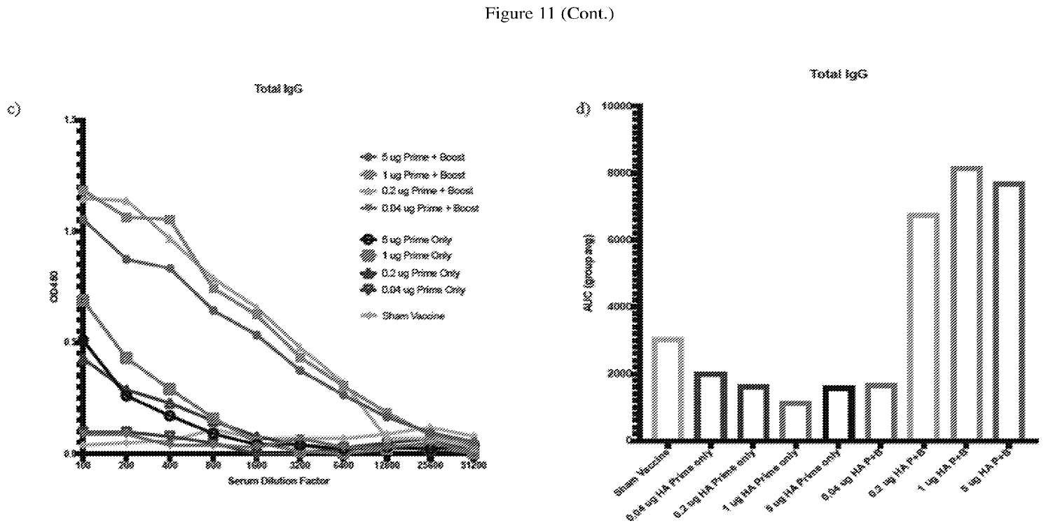

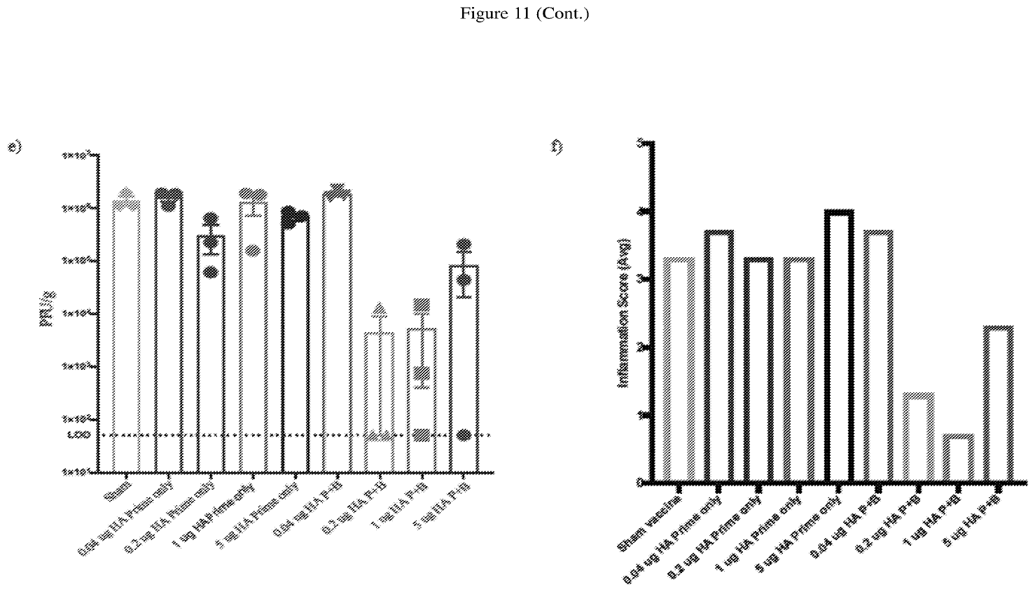

To determine the concentration of HA needed for vaccinations that allow for observation of adjuvant effects, a concentration curve of antigen alone in both prime only (FIG. 11A) or prime-boost (FIG. 11B) vaccinations were performed in mice (n=3). To assess the immune response in the vaccinated mice, total IgG titers (FIG. 11C) were measured. This revealed a low, but elevated, antibody production above sham for the 0.04 ug prime-boost group while the 0.2 ug (p=0.0054), 1.0 ug (p=0.0043), and 5.0 ug (p=0.004) rHA groups demonstrated high levels of anti-rHA antibody responses (FIG. 11D). On day 28 mice were challenged by intranasal inoculation of Influenza virus and evaluated daily for protection from weight loss. None of the prime only vaccination concentrations provided protection from weight loss after challenge. When administered in a prime-boost schedule; 0.2 ug, 1.0 ug, and 5.0 ug of rHA alone were protective against significant weight loss while the mice vaccinated with 0.04 ug HA had significant weight loss. Plaque assays were used to measure plaque forming units per gram (PFU/g) of day 7 post-challenge mouse lung homogenate (FIG. 11E). Similar to antibody titer results, all prime only groups and the 0.04 ug prime-plus boost group had high virus levels in the lungs similar to the sham vaccination group. Vaccination groups that received 0.2, 1.0, and 5.0 ug prime-boost injections had one to two logs lower viral titer than the 0.04 ug prime plus boost group. Pathological scoring was performed on H&E stained lungs and inflammation scores calculated. All groups showed significant inflammation however the 0.2, 1.0, and 5.0 ug prime-boost groups showed minimal inflammation similar to the sham infection group (FIG. 11F). These data show that 0.04 ug HA alone with a prime-boost vaccination schedule is not protective. However, low-level antibody titers elicited in this group suggest that this concentration does elicit an immune response that can potentially be boosted to a level that affords protection from challenge when an adjuvant is added.

Adjuvant Immune Stimulation and Protection from Homologous Challenge

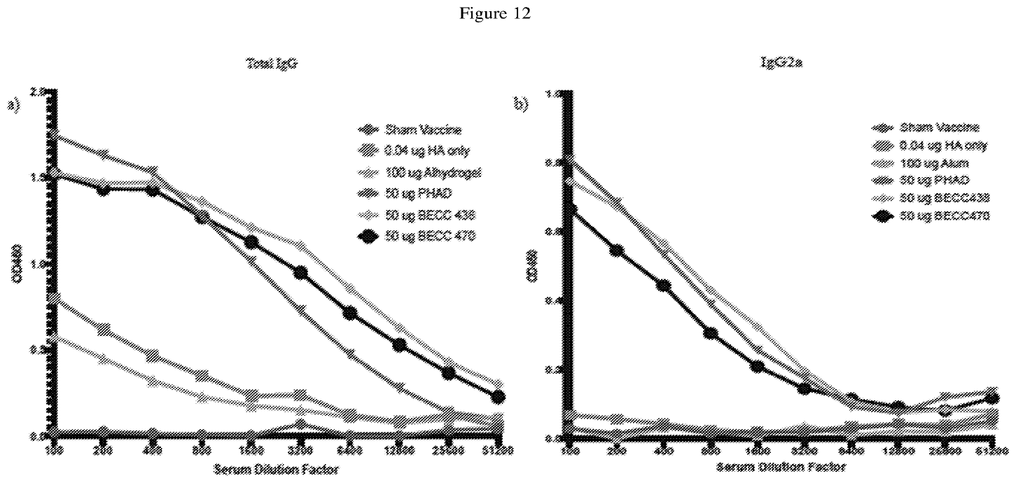

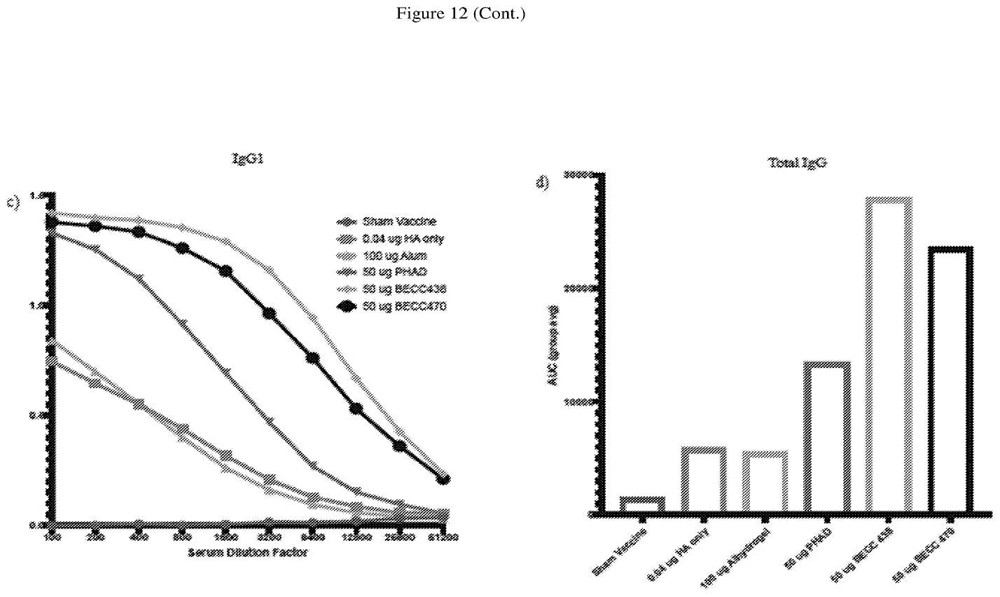

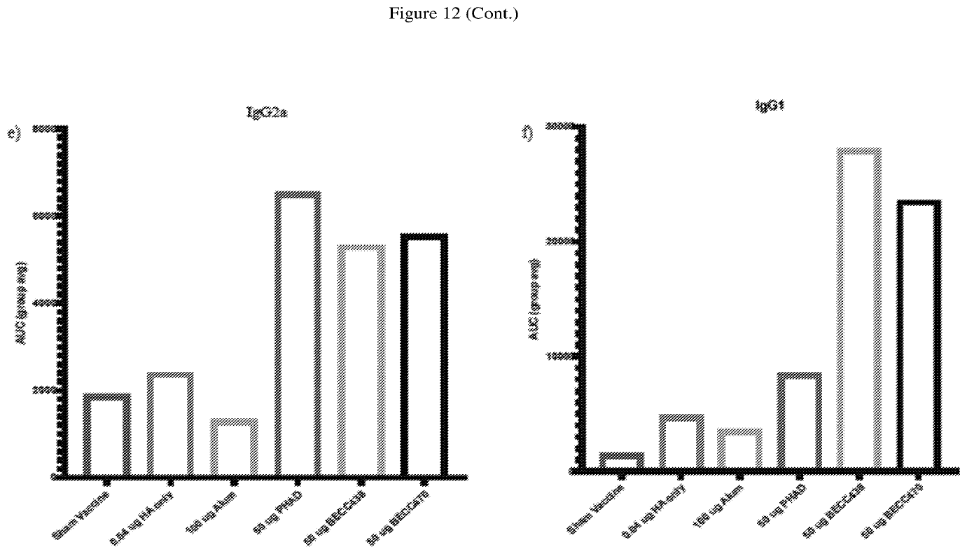

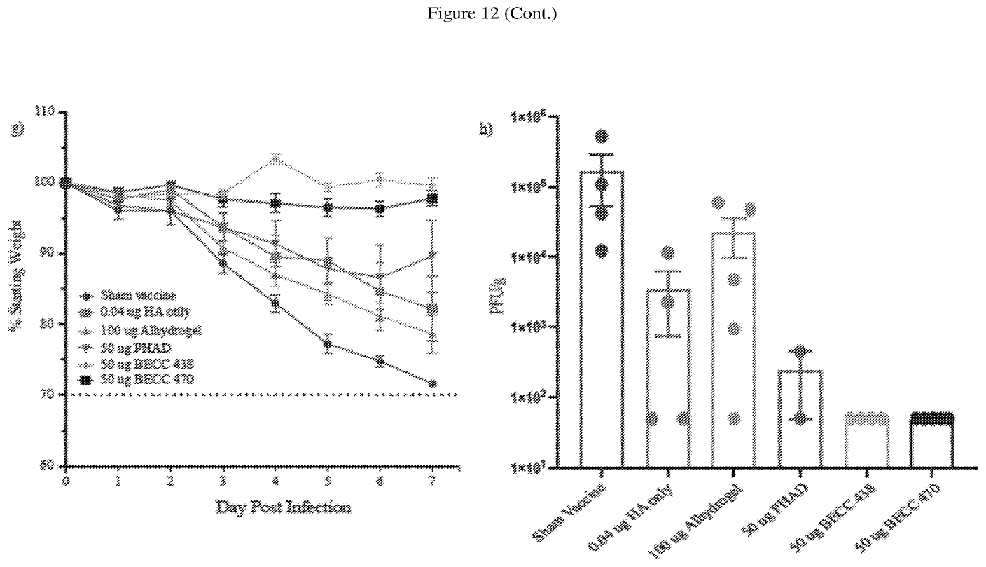

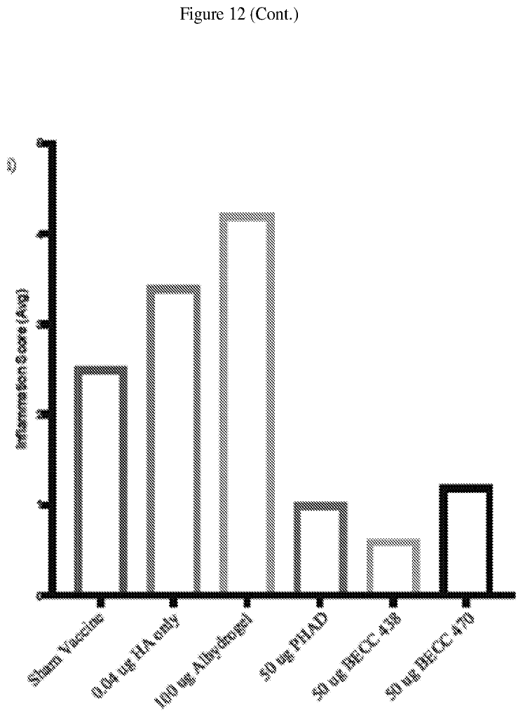

To assess protection from Influenza virus in a homologous challenge model where the antigen is derived from the same strain used for infection, mice were immunized with 0.04 ug rHA adsorbed to either, 100 ug Alum, 50 ug PHAD, 50 ug BECC438, or 50 ug BECC470 along with sham and HA only control groups in a prime (day 0)-boost (day 14) schedule. Total serum IgG from Day 28 shows that while there is a significant difference between the HA alone and PHAD groups (p=0.0016), the BECC438 and BECC470 adjuvanted groups have greatly increased IgG antibody titers when compared to HA alone (p<0.0001)(FIGS. 12A and 12D). HA-specific IgG2a (FIG. 12B) and IgG1 (FIG. 12C) titers were also measured to assess the Th1 and Th2-type immune response respectively. While antigen alone and alum adjuvanted groups did not show IgG2a production above sham vaccinated levels, IgG2a was significantly higher than sham and comparable among the PHAD (p=0.0066), BECC438 (p=0.0061), and 470 (p=0.0058) adjuvanted groups (FIG. 12E). IgG1 production, however, is similar to sham for HA only, Alum and PHAD groups, while BECC438 (p<0.0001) and 470 (p=0.0001) were both significantly elevated (FIG. 12F). Following challenge with IAV NL/09, marked average weight loss is observed as nearly 30% in the sham group, about 20% for the HA only and Alum groups, 10% in the PHAD group, while the BECC adjuvanted groups lost little weight (FIG. 12G). Virus titer values correspond with weight loss showing that both BECC adjuvanted groups had no detectable viral titer in lungs (FIG. 12H). Histological inflammation scoring demonstrates that the sham infected group on average is markedly lower than that of both the HA only and Alum groups with prominent to diffuse bronchiolar and periarterial inflammation with bronchial necrosis in these latter two groups. The PHAD and BECC groups have low inflammation scores with minimal bronchiolar or periarterial inflammation (FIG. 12I). Compared to control groups, BECC adjuvants elicit a balanced Th1/Th2-type immune response, and provide superior protection from weight loss, virus replication, and lung pathology when challenged with a homologous IAV strain.

### Adjuvant Sparing Capability Observed for BECC438 and BECC470

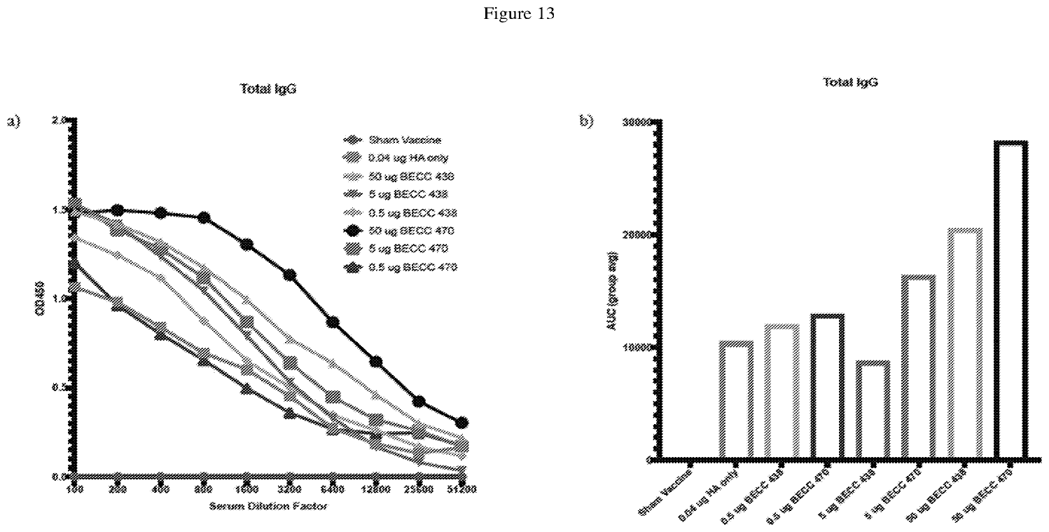

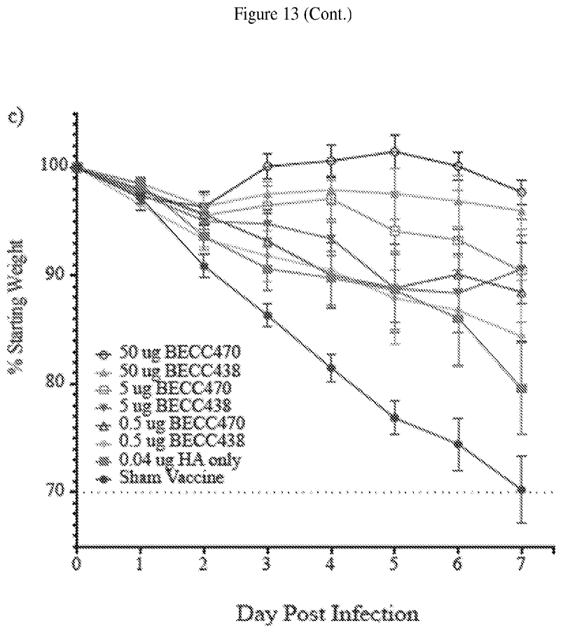

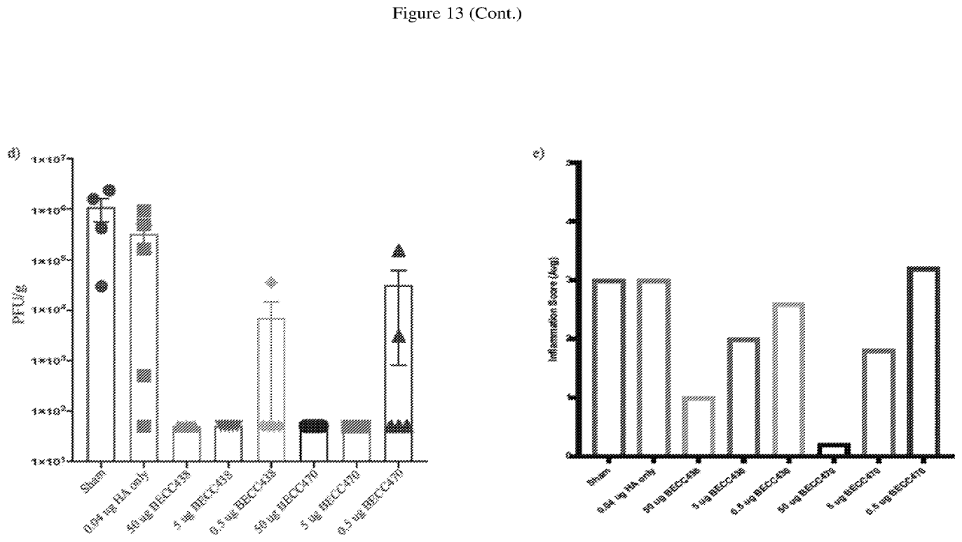

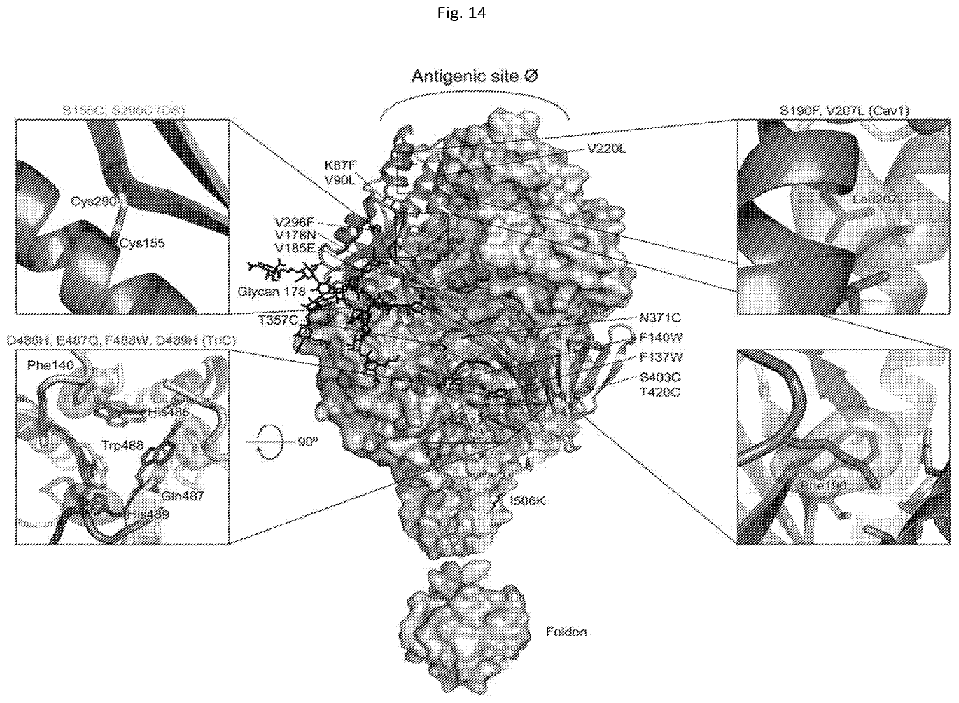

To determine a minimal rHA antigen level when formulated with the BECC adjuvants, mice were immunized with 0.04 ug rHA in combination with either, 50, 5 or 0.5 ug of BECC438 or BECC470 along with sham and HA only control groups in a prime-boost schedule. At day 28 post vaccination, total IgG antibody production was determined. We found that total IgG production is below detection in sham mice and moderately elevated in the groups with HA only, 0.5 ug BECC438, 0.5 ug BECC470, and 5 ug BECC438. Significantly higher antibody titers are observed when adjuvanted with 5 ug BECC470 (p=0.0015), 50 ug BECC438 (p=0.0002), and 50 ug BECC470 (p=0.0001) (FIGS. 13A and 13B). In virus challenged mice, average observed weight loss was similar as before in control groups, 10-15% in the 0.5 ug BECC groups, 8% in the 5 ug BECC groups and negligible weight loss in the 50 ug BECC groups (FIG. 13C). Virus titer is approximately 1×10⁶ PFU/g in the sham group, whereas the HA only group had around a half-log lower virus titer. Both 0.5 ug BECC adjuvanted groups have approximately two logs lower virus titer than sham and mouse lungs from all 5 and 50 ug BECC groups displayed no plaques (FIG. 13D). Histological inflammation scoring shows that the sham group on average is similar to the HA only, and both 0.5 ug BECC groups. Pathology in these groups show moderate to prominent bronchiolar and periarterial inflammation with bronchial necrosis. Histological scoring also shows dose-dependence with 5 ug BECC groups decreased inflammation scores and correspondingly minimal to prominent bronchiolar or periarterial inflammation. The 50 ug BECC groups show very low to negligible inflammation and scant or zero bronchiolar or periarterial inflammation (FIG. 13E).

BECC Adjuvant: rHA Vaccine Protect Mice with a Single Dose

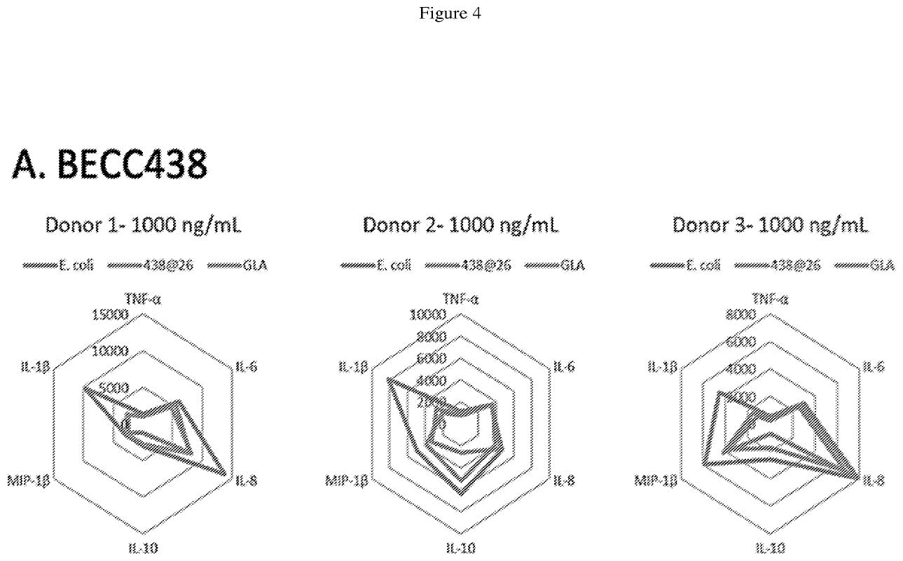

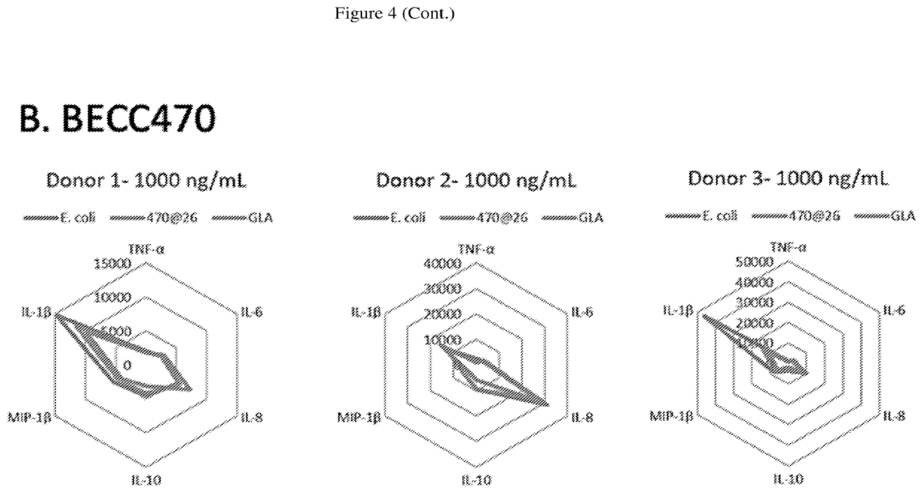

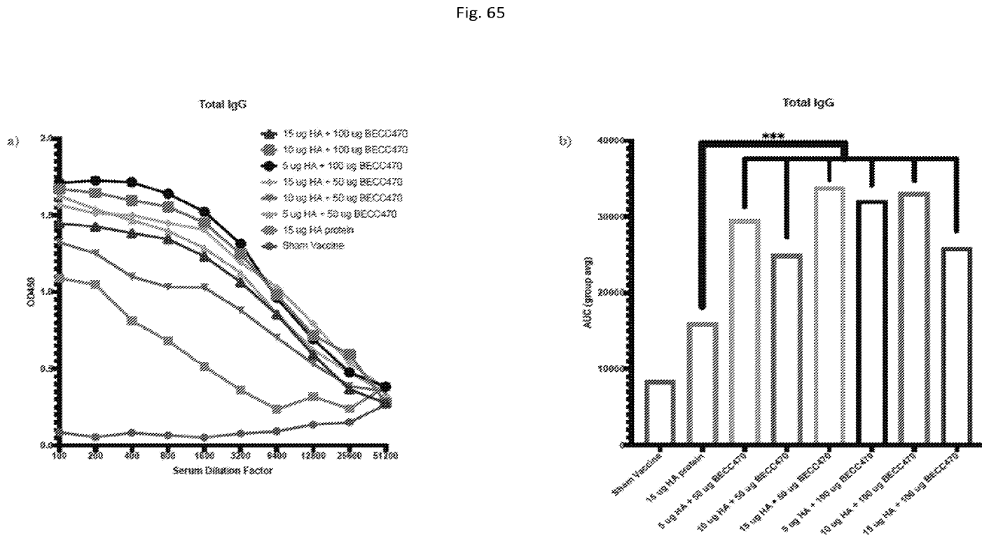

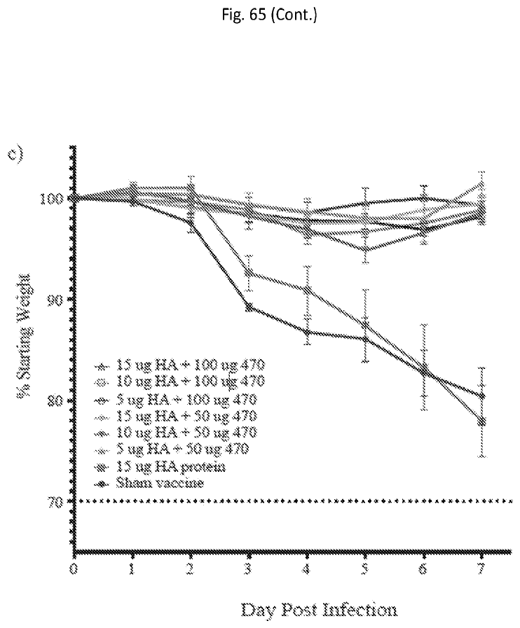

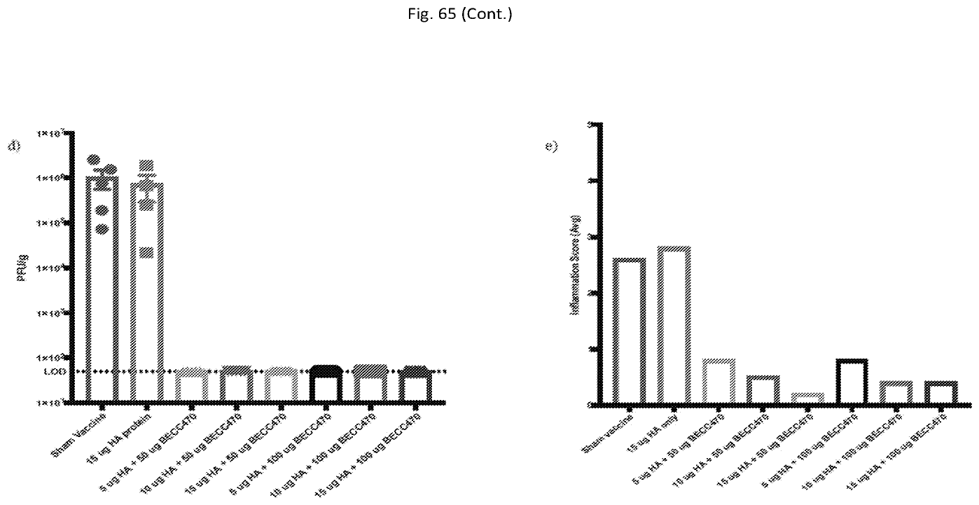

Current seasonal Influenza vaccines are administered in a single dose, and so we next wanted to determine if a prime-alone BECC adjuvanted vaccine provides protection from homologous IAV challenge. We chose to use BECC470 because it has shown slightly better performance over BECC438 in our previous studies. Mice were immunized with 5, 10 or 15 ug rHA in combination with either 50 or 100 ug of BECC470 along with sham and 15 ug rHA only control groups with a single prime dose. ELISA titers were quantified and we find that total IgG antibody production is very low in sham mice and with significantly higher titers in all other groups compared to HA alone (p<0.001) (FIGS. 65A and 65B). The difference in weight loss between the sham and HA only groups and all others, however, is striking. The HA only group is similar to sham where they show no protection from the challenge. All other groups including the lowest HA (5 ug) and BECC470 (50 ug) groups demonstrate a near complete protection from weight loss (FIG. 4C). Virus titer is approximately 1×10⁶ PFU/g in both the sham and HA only groups. The BECC470 adjuvanted groups, however, showed no plaques in our assays (FIG. 65D). Histological inflammation scoring shows that the sham group, on average, is similar to the HA only group and pathology in these groups shows prominent bronchiolar and periarterial inflammation with mild bronchial necrosis. Inflammation scoring in all BECC470 adjuvanted groups, corresponding to viral titer, shows low inflammation scores with zero to scant bronchiolar and periarterial inflammation (FIG. 65E).

Extending Protection from Divergent Strain

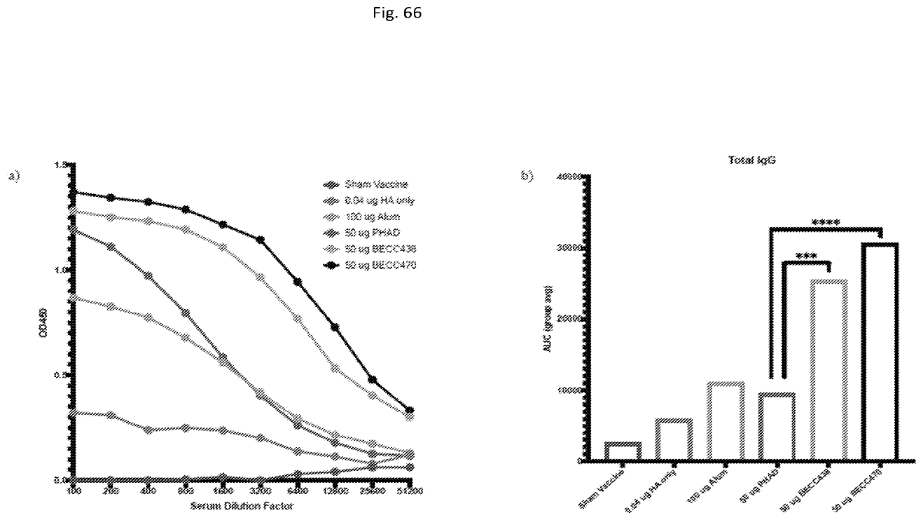

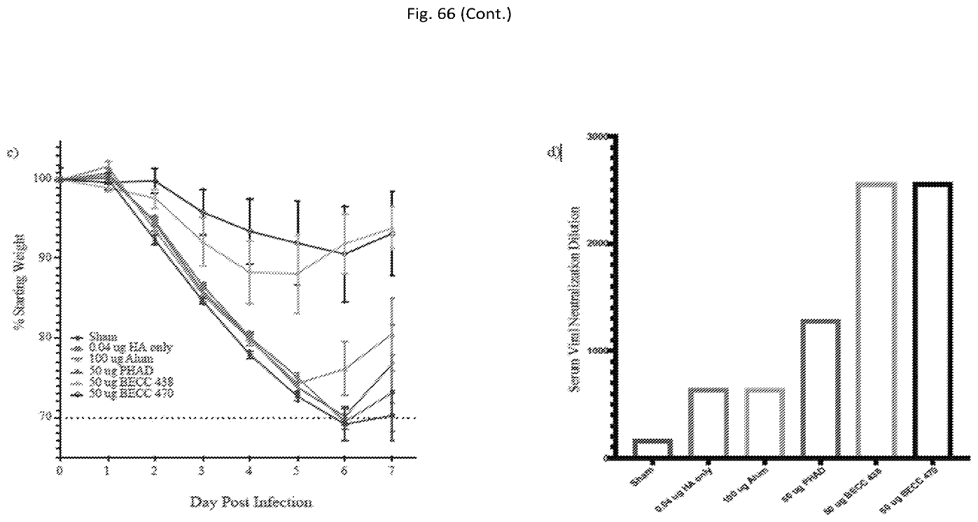

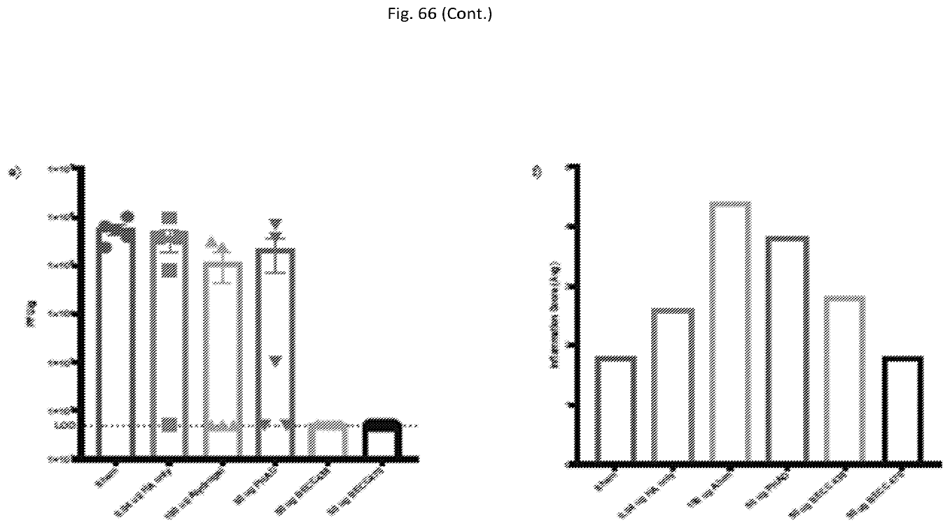

Due to the poor genomic replication fidelity of the Influenza virus and antigenic drift, especially of the highly immunogenic HA surface protein, there is a requirement for a seasonal vaccine. A vaccine that provides protection across a broad array of antigenically variant HA proteins and therefore divergent Influenza strains could improve vaccination efficacy rates and reduce disease prevalence. To test immune protection against a heterologous Sing/2015 challenge, we formulated the same vaccinations as those used in the homologous NL/09 challenge. Mice were immunized with 0.04 ug rHA in combination with either, 100 ug Alum, 50 ug PHAD, 50 ug BECC438 or 50 ug BECC470 along with sham and HA only control groups with a prime-boost schedule. Similar to the mice in the homologous challenge experiment, Day 28 serum total IgG antibody production is negligible in sham mice, minimally elevated in the HA only group and somewhat higher in the HA in combination with Alum or PHAD as adjuvants. The BECC438 (p=0.0002) and BECC470 (p<0.0001) adjuvanted groups, however, show significantly higher IgG antibody titer, even when compared to PHAD (FIGS. 66A and 66B). Weight loss in control groups was similar to homologous challenge mice, 30% from starting weight in the sham group and at least 20% for the HA only, Alum and PHAD groups. However, both BECC adjuvanted groups only lost 7% of starting weight (FIG. 66C) which correlated with superior viral neutralization assay titers (FIG. 66D). Detected virus titer for both BECC groups were also at or below the limit of detection (FIG. 66E) and histological inflammation scoring shows only moderate inflammation with scant to diffuse and mild to moderate bronchiolar or periarterial inflammation (FIG. 66F).

### Bacterial Enzymatic Combinatorial Chemistry Mutagenesis

As previously reported [21], BECC438 is extracted from mutant bacterial strains created by mating Y. pestis KIM6⁺⊗msbB with E. coli conjugation donor strain S17-1Lpir containing the plasmid, pCVD442-pagP_{Yp}^{ReP}. Strains were grown on selective media and sequenced to confirm the presence of the pagP gene. BECC470 is created similarly by mating Yp KIM6⁺ with E. coli containing plasmids lpxF and pagP [15]. Lipooligosaccharide (LOS) was isolated from the mutants, purified, and the structure was confirmed by mass spectrometry and gas chromatography.

### Murine Immunization and Influenza Virus Challenge

For all experiments, except for immunization and challenge of elderly mice, we used six to eight-week-old, female, WT, BALB/c mice purchased from Charles River Laboratories (Wilmington, Mass.). Elderly mice were 10 to 12-month-old, non-pregnant, WT, retired breeders purchased from Taconic Biosciences (Germantown, N.Y.). All experiments were approved by the University of Maryland, Baltimore (UMB) Institutional Animal Care and Use Committee, protocol #0318006. Mice were prime immunized on day 0 and boosted on day 14 intramuscularly in the right caudal thigh with 50 uL volume of vaccine solution. Up to 200 uL of blood were harvested from the periorbital vein at days 0, 14 and 28. Whole blood was centrifuged in serum separator tubes (#365967, Becton Dickinson, Franklin Lakes, N.J.) and sera was then aliquoted and stored at −80° C. for immune titer determination via enzyme-linked immunosorbent assay (ELISA).

To create the vaccine inoculum, adjuvant molecules were resuspended in water at the indicated concentrations and incubated in a bath sonicator for 15 min to promote micelle formation. Hemagglutinin (HA) antigen in the indicated concentration was added to the vaccine solution and allowed to adsorb for 2 hours at room temperature. This component vaccine was created fresh for each vaccination and used immediately after adsorption.

All mice were challenged on day 28 of the experiment. For all challenge experiments except for the heterologous challenge, mice were inoculated intranasally with 3200 PFU of A/Netherlands/602/2009 (H1N1) (NL/09) virus and weighed daily for seven days. Similarly, the heterologous challenge mice were inoculated intranasally with 51 PFU of A/Singapore/GP1908/2015 (H1N1)(Sing/2015) virus and weighed for seven days (both viruses provided by Krammer Lab, Mount Sinai Hospital, New York, N.Y.). Mice that fell below 70% of starting weight were euthanized. On day seven mice were harvested for blood and lung tissue. While the NL/09 and Sing/2015 strains are both Influenza A viruses, phylogenetically there is a 14 AA substitution difference between the two strains.

### HA Specific Antibody Titers by ELISA

Recombinant HA was prepared as previously described [22]. Briefly, rHA antigens derived from influenza virus A/California/04/2009 (pandemic H1N1) (Cal/09) were expressed from baculovirus vectors in High Five cells as soluble trimers, by utilizing the T4 phage fibritin natural trimerization domain and a C-terminal hexahistidine tag for purification. Protein was purified with Ni-nitrilotriacetic acid (NTA)-agarose beads. Cal/09 and NL/09 have previously been shown [25] to be phylogenetically matched HA proteins where antibody to the NL/09 virus has been shown to be reactive to Cal/09 protein. Sera collected on day 28 (pre-infection) of the vaccination schedule was used to evaluate antibody titer by ELISA. Cal/09 rHA coating protein (Krammer Lab) was diluted in coating buffer (Bio-Rad Indirect ELISA Protocol) at a concentration of 2 ug/mL and 50 uL of this solution was added to each well of a 96-well flat-bottomed plate overnight at 4° C. (Nunc Maxisorp #44240421). Plates were washed three times with PBS containing 0.1% Tween 20 (PBS-T) before blocking all wells for 1 hour at room temperature with 220 uL of PBS-T/3% goat serum/0.5% milk. Blocking buffer was then removed and 100 uL of serum, diluted in blocking buffer, was added in a ten point, two-fold dilution series starting with a 1:100 dilution. The first and last columns are left as serum-free negative controls and all serum specimens are run in duplicate. Once on the plate samples were incubated at room temperature for two hours. Plates are washed three times with PBS-T and 50 uL of 1:3000 dilution of secondary antibody for either total IgG (goat anti-mouse IgG, KPL-474-1802, Kirkegaard and Perry Laboratories, Gaithersburg, Md.), IgG1 (1070-05, Southern Biotech, Birmingham, Ala.) or IgG2a (1080-05, Southern Biotech, Birmingham, Ala.) is added to all wells for one hour at room temperature. Plates were then washed four times with PBS-T. 50 uL of room temperature BD OptEIA TMB substrate (BD Biosciences, San Jose, Calif.) was added and the plates were incubated for 10 minutes and the reaction stopped by adding 50 uL of 3M HCl. An OD₄₅₀ value was determined using a BioTek Instruments Synergy HTX plate reader using BioTek Instruments Gen5 software (BioTek Instruments, Winooski, Vt.). All analyses and graphs were made using Prism 8 for Mac OS X (GraphPad Software, Inc., La Jolla, Calif.). Antibody titer and Area Under the Curve (AUC) are averaged by vaccination group with the average plate blank subtracted from the readout.

### Lung Tissue Histology

Staining of lung tissue was performed by the UMB EM and Histology Laboratory to assess any histological signs and potential clearing of viral infection. 4% Paraformaldehyde-fixed (Sigma-Aldrich, St Louis, Mo.) lungs were embedded in paraffin and sectioned before staining with haematoxylin and eosin (H&E). Representative slides were selected to most accurately portray the condition of the lungs within each group of vaccinated mice. Pathology scoring was performed by Dr (LT) Robert Kitz, Anatomic Pathologist, Walter Reed National Military Medical Center, Bethesda, Md. Lung sections from each mouse were scored based on overall inflammation using the following scoring parameters (group scores averaged): 0=no inflammation, 1=scant inflammation (<5% multifocal or one small focus); 2=prominent inflammation, <25% parenchyma; 3=prominent inflammation, 25-50% parenchyma; 4=diffuse inflammation, 50-75% parenchyma; 5=diffuse inflammation, >75% parenchyma.

### Plaque Assays for Lung Virus Titer

Plaque assays were used to determine the lung IAV titers from mice sacrificed on day 7 post-infection. Madin-Darby Canine Kidney (MDCK) cells were grown to confluence overnight at 37° C. on 6-well plates and a dilution series from 1×10⁻¹-1×10⁻⁶ of lung homogenate were added to the wells and allowed to infect the cells for 1 hour with period rocking. After one hour, cells were washed with DMEM (Quality Biological, Gaithersburg, Md.) supplemented with 1% Penicillin/Streptomycin (Gemini Bio-Products, Sacramento, Calif.) and 1% L-glutamine (Gibco/Life Technologies, Grand Island, N.Y.). A 3-mL overlay of SeaKem LE Agarose (Lonza, Rockland, Me.) mixed with 1:500 dilution of TPCK-treated trypsin (Sigma-Aldrich, St Louis, Mo.) in 2×MEM (Quality Biological, Gaithersburg, Md.) was used to cover the cells in each well and the plates were incubated at 37° C. for three days. After this incubation period the agarose was removed, cells were fixed with 70% ethanol then stained with 0.05% crystal violet. Plaques were visualized and counted manually.

### Virus Neutralization Assay

Virus neutralization assays were used to determine the protective capacity of antibody to inhibit viral cell entry and replication. MDCK cells were grown to confluence overnight at 37° C. on a 96-well flat-bottomed plate. In a 96-well round-bottomed plate 25 uL of day 28 pooled serum from each vaccination group was diluted of 1:10 in the first column of the plate and then 1:2 across the remainder of the plate in DMEM supplemented with 1% Penicillin/Streptomycin and 1% L-glutamine then treated with an addition of 1:1000 dilution of TPCK-treated trypsin. This is the serum dilution plate. In a series of separate dilutions, Sing/2015 Influenza was diluted to 100 PFU/50 uL also in DMEM supplemented media with trypsin. 60 uL from each well of the serum dilution plate was moved row-by-row into rows A-F in a 96-well round-bottomed plate. 60 uL of diluted Sing/15 virus was added to all wells of rows A-G. 60 uL of DMEM supplemented media with trypsin was added to row G and 120 uL to row H. This is the serum/virus plate. The plate was then allowed to incubate at room temperature for one hour on a shaker. After one hour, growth media was removed from the cell plate and washed twice with 200 uL of PBS. 100 uL from the serum/virus plate was transferred row-by-row into rows A-H of the cell plate and allowed to incubate at 37° C. for one hour. Afterward, the supernatant was removed, and all wells were washed with 200 uL of PBS to stop the infection. 100 uL of diluted serum was added to rows A-F of the cell plate and 100 uL of DMEM supplemented media with trypsin was added to rows G & H. This plate was incubated at 37° C. for 72 hours then all wells were fixed for 10 minutes with 70% ethanol and stained for 10 minutes with 0.05% crystal violet. The plate was read manually for neutralization.

### Methods

Bacterial Enzymatic Combinatorial Chemistry Mutagenesis

As previously reported [21], BECC438 is extracted from mutant bacterial strains created by mating E pestis KIM6⁺ΔmsbB with E. coli conjugation donor strain S17-1λpir containing the plasmid, pCVD442-pag_{YP}^{Rep}. Strains were grown on selective media and sequenced to confirm the presence of the pagP gene. BECC470 is created similarly by mating Yp KIM6⁺ with E. coli containing plasmids lpxF and pagP [15]. Lipooligosaccharide (LOS) was isolated from the mutants, purified, and the structure was confirmed by mass spectrometry and gas chromatography.

Murine Immunization and Influenza Virus Challenge

For all experiments, except for immunization and challenge of elderly mice, we used six to eight-week-old, female, WT, BALB/c mice purchased from Charles River Laboratories (Wilmington, Mass.). Elderly mice were 10 to 12-month-old, non-pregnant, WT, retired breeders purchased from Taconic Biosciences (Germantown, N.Y.). All experiments were approved by the University of Maryland, Baltimore (UMB) Institutional Animal Care and Use Committee, protocol #0318006. Mice were prime immunized on day 0 and boosted on day 14 intramuscularly in the right caudal thigh with 50 uL volume of vaccine solution. Up to 200 uL of blood were harvested from the periorbital vein at days 0, 14 and 28. Whole blood was centrifuged in serum separator tubes (#365967, Becton Dickinson, Franklin Lakes, N.J.) and sera was then aliquoted and stored at −80° C. for immune titer determination via enzyme-linked immunosorbent assay (ELISA).

To create the vaccine inoculum, adjuvant molecules were resuspended in water at the indicated concentrations and incubated in a bath sonicator (manufacturer/model number) for 15 min to promote micelle formation. Hemagglutinin (HA) antigen in the indicated concentration was added to the vaccine solution and allowed to adsorb for 2 hours at room temperature. This component vaccine was created fresh for each vaccination and used immediately after adsorption.

All mice were challenged on day 28 of the experiment. For all challenge experiments except for the heterologous challenge, mice were inoculated intranasally with 3200 PFU of A/Netherlands/602/2009 (H1N1) (NL/09) virus and weighed daily for seven days. Similarly, the heterologous challenge mice were inoculated intranasally with 51 PFU of A/Singapore/GP1908/2015 (H1N1)(Sing/2015) virus and weighed for seven days (both viruses provided by Krammer Lab, Mount Sinai Hospital, New York, N.Y.). Mice that fell below 70% of starting weight were euthanized. On day seven mice were harvested for blood and lung tissue. While the NL/09 and Sing/2015 strains are both Influenza A viruses, phylogenetically there is a 14 AA substitution difference between the two strains. In (reference) Sing/15 has been shown to illicit no antibody response in a murine Cal/09 vaccination.

### HA Specific Antibody Titers by ELISA

Recombinant HA was prepared as previously described [22]. Briefly, rHA antigens derived from influenza virus A/California/04/2009 (pandemic H1N1) (Cal/09) were expressed from baculovirus vectors in High Five cells as soluble trimers, by utilizing the T4 phage fibritin natural trimerization domain and a C-terminal hexahistidine tag for purification. Protein was purified with Ni-nitrilotriacetic acid (NTA)-agarose beads. Cal/09 and NL/09 have previously been shown [25] to be phylogenetically matched HA proteins where antibody to the NL/09 virus has been shown to be reactive to Cal/09 protein. Sera collected on day 28 (pre-infection) of the vaccination schedule was used to evaluate antibody titer by ELISA. Cal/09 rHA coating protein (Krammer Lab) was diluted in coating buffer (Bio-Rad Indirect ELISA Protocol) at a concentration of 2 ug/mL and 50 uL of this solution was added to each well of a 96-well flat-bottomed plate overnight at 4° C. (Nunc Maxisorp #44240421). Plates were washed three times with PBS containing 0.1% Tween 20 (PBS-T) before blocking all wells for 1 hour at room temperature with 220 uL of PBS-T/3% goat serum/0.5% milk. Blocking buffer was then removed and 100 uL of serum, diluted in blocking buffer, was added in a ten point, two-fold dilution series starting with a 1:100 dilution. The first and last columns are left as serum-free negative controls and all serum specimens are run in duplicate. Once on the plate samples were incubated at room temperature for two hours. Plates are washed three times with PBS-T and 50 uL of 1:3000 dilution of secondary antibody for either total IgG (goat anti-mouse IgG, KPL-474-1802, Kirkegaard and Perry Laboratories, Gaithersburg, Md.), IgG1 (1070-05, Southern Biotech, Birmingham, Ala.) or IgG2a (1080-05, Southern Biotech, Birmingham, Ala.) is added to all wells for one hour at room temperature. Plates were then washed four times with PBS-T. 50 uL of room temperature BD OptEIA TMB substrate (BD Biosciences, San Jose, Calif.) was added and the plates were incubated for 10 minutes and the reaction stopped by adding 50 uL of 3M HCl. An OD₄₅₀ value was determined using a BioTek Instruments Synergy HTX plate reader using BioTek Instruments Gen5 software (BioTek Instruments, Winooski, Vt.). All analyses and graphs were made using Prism 8 for Mac OS X (GraphPad Software, Inc., La Jolla, Calif.). Antibody titer and Area Under the Curve (AUC) are averaged by vaccination group with the average plate blank subtracted from the readout.

### Lung Tissue Histology

Staining of lung tissue was performed by the UMB EM and Histology Laboratory to assess any histological signs and potential clearing of viral infection. 4% Paraformaldehyde-fixed (Sigma-Aldrich, St Louis, Mo.) lungs were embedded in paraffin and sectioned before staining with haematoxylin and eosin (H&E). Representative slides were selected to most accurately portray the condition of the lungs within each group of vaccinated mice. Pathology scoring was performed by Dr (LT) Robert Kitz, Anatomic Pathologist, Walter Reed National Military Medical Center, Bethesda, Md. Lung sections from each mouse were scored based on overall inflammation using the following scoring parameters (group scores averaged): 0=no inflammation, 1=scant inflammation (<5% multifocal or one small focus); 2=prominent inflammation, <25% parenchyma; 3=prominent inflammation, 25-50% parenchyma; 4=diffuse inflammation, 50-75% parenchyma; 5=diffuse inflammation, >75% parenchyma.

### Plaque Assays for Lung Virus Titer

Plaque assays were used to determine the lung IAV titers from mice sacrificed on day 7 post-infection. Madin-Darby Canine Kidney (MDCK) cells were grown to confluence overnight at 37° C. on 6-well plates and a dilution series from 1×10⁻¹-1×10⁻⁶ of lung homogenate were added to the wells and allowed to infect the cells for 1 hour with period rocking. After one hour, cells were washed with DMEM (Quality Biological, Gaithersburg, Md.) supplemented with 1% Penicillin/Streptomycin (Gemini Bio-Products, Sacramento, Calif.) and 1% L-glutamine (Gibco/Life Technologies, Grand Island, N.Y.). A 3-mL overlay of SeaKem LE Agarose (Lonza, Rockland, Me.) mixed with 1:500 dilution of TPCK-treated trypsin (Sigma-Aldrich, St Louis, Mo.) in 2×MEM (Quality Biological, Gaithersburg, Md.) was used to cover the cells in each well and the plates were incubated at 37° C. for three days. After this incubation period the agarose was removed, cells were fixed with 70% ethanol then stained with 0.05% crystal violet. Plaques were visualized and counted manually.

### Virus Neutralization Assay

Virus neutralization assays were used to determine the protective capacity of antibody to inhibit viral cell entry and replication. MDCK cells were grown to confluence overnight at 37° C. on a 96-well flat-bottomed plate. In a 96-well round-bottomed plate 25 uL of day 28 pooled serum from each vaccination group was diluted of 1:10 in the first column of the plate and then 1:2 across the remainder of the plate in DMEM supplemented with 1% Penicillin/Streptomycin and 1% L-glutamine then treated with an addition of 1:1000 dilution of TPCK-treated trypsin. This is the serum dilution plate. In a series of separate dilutions, Sing/2015 Influenza was diluted to 100 PFU/50 uL also in DMEM supplemented media with trypsin. 60 uL from each well of the serum dilution plate was moved row-by-row into rows A-F in a 96-well round-bottomed plate. 60 uL of diluted Sing/15 virus was added to all wells of rows A-G. 60 uL of DMEM supplemented media with trypsin was added to row G and 120 uL to row H. This is the serum/virus plate. The plate was then allowed to incubate at room temperature for one hour on a shaker. After one hour, growth media was removed from the cell plate and washed twice with 200 uL of PBS. 100 uL from the serum/virus plate was transferred row-by-row into rows A-H of the cell plate and allowed to incubate at 37° C. for one hour. Afterward, the supernatant was removed, and all wells were washed with 200 uL of PBS to stop the infection. 100 uL of diluted serum was added to rows A-F of the cell plate and 100 uL of DMEM supplemented media with trypsin was added to rows G & H. This plate was incubated at 37° C. for 72 hours then all wells were fixed for 10 minutes with 70% ethanol and stained for 10 minutes with 0.05% crystal violet. The plate was read manually for neutralization.

### LITERATURE (THIS EXAMPLE)

1. Molinari, N. A., et al., The annual impact of seasonal influenza in the US: measuring disease burden and costs. Vaccine, 2007. 25(27): p. 5086-96.
2. Taubenberger, J. K. and D. M. Morens, 1918 Influenza: the mother of all pandemics. Emerg Infect Dis, 2006. 12(1): p. 15-22.
3. Reid, A. H. and J. K. Taubenberger, The 1918 flu and other influenza pandemics: “over there” and back again. Lab Invest, 1999. 79(2): p. 95-101.
4. Suarez, D. L., et al., Recombination resulting in virulence shift in avian influenza outbreak, Chile. Emerg Infect Dis, 2004. 10(4): p. 693-9.
5. Treanor, J., Influenza vaccine—outmaneuvering antigenic shift and drift. N Engl J Med, 2004. 350(3): p. 218-20.
6. Xu, X., et al., Update: Influenza Activity in the United States During the 2018-19 Season and Composition of the 2019-20 Influenza Vaccine. MMWR Morb Mortal Wkly Rep, 2019. 68(24): p. 544-551.
7. Nelson, M. I. and E. C. Holmes, The evolution of epidemic influenza. Nat Rev Genet, 2007. 8(3): p. 196-205.
8. Wiley, S. K., Seasonal influenza vaccine guidelines, 2018-2019. Nursing, 2018. 48(11): p. 11-13.
9. Campos-Outcalt, D., CDC recommendations for the 2018-2019 influenza season. J Fam Pract, 2018. 67(9): p. 550-553.
10. Fluad—an adjuvanted seasonal influenza vaccine for older adults. Med Lett Drugs Ther, 2016. 58(1486): p. 8.
11. Cruz-Valdez, A., et al., MF59-adjuvanted influenza vaccine (FLUAD(R)) elicits higher immune responses than a non-adjuvanted influenza vaccine (Fluzone(R)): A randomized, multicenter, Phase III pediatric trial in Mexico. Hum Vaccin Immunother, 2018. 14(2): p. 386-395.
12. Ko, E. J. and S. M. Kang, Immunology and efficacy of MF59-adjuvanted vaccines. Hum Vaccin Immunother, 2018. 14(12): p. 3041-3045.
13. Ansaldi, F., et al., Cross protection by MF59-adjuvanted influenza vaccine: neutralizing and haemagglutination-inhibiting antibody activity against A(H3N2) drifted influenza viruses. Vaccine, 2008. 26(12): p. 1525-9.
14. Ko, E. J., et al., MPL and CpG combination adjuvants promote homologous and heterosubtypic cross protection of inactivated split influenza virus vaccine. Antiviral Res, 2018. 156: p. 107-115.
15. Gregg, K. A., et al., Rationally Designed TLR4 Ligands for Vaccine Adjuvant Discovery. MBio, 2017. 8(3).
16. Martinon, S., et al., Chemical and Immunological Characteristics of Aluminum-Based, Oil-Water Emulsion, and Bacterial-Origin Adjuvants. J Immunol Res, 2019. 2019: p. 3974127.
17. Hem, S. L. and J. L. White, Structure and properties of Aluminum-containing adjuvants. Pharm Biotechnol, 1995. 6: p. 249-76.
18. Ghimire, T. R., The mechanisms of action of vaccines containing Aluminum adjuvants: an in vitro vs in vivo paradigm. Springerplus, 2015. 4: p. 181.
19. Gao, J. and Z. Guo, Progress in the synthesis and biological evaluation of lipid A and its derivatives. Med Res Rev, 2018. 38(2): p. 556-601.
20. Hernandez, A., et al., Phosphorylated Hexa-Acyl Disaccharides Augment Host Resistance Against Common Nosocomial Pathogens. Crit Care Med, 2019. 47(11): p. e930-e938.
21. Gregg, K. A., et al., A lipid A-based TLR4 mimetic effectively adjuvants a Yersinia pestis rF-V1 subunit vaccine in a murine challenge model. Vaccine, 2018. 36(28): p. 4023-4031.
22. Goff, P. H., et al., Synthetic Toll-Like Receptor 4 (TLR4) and TLR7 Ligands Work Additively via MyD88 To Induce Protective Antiviral Immunity in Mice. J Virol, 2017. 91(19).
23. Cox, M. M. and J. R. Hollister, FluBlok, a next generation influenza vaccine manufactured in insect cells. Biologicals, 2009. 37(3): p. 182-9.
24. King, J. C., Jr., et al., Evaluation of the safety, reactogenicity and immunogenicity of FluBlok trivalent recombinant baculovirus-expressed hemagglutinin influenza vaccine administered intramuscularly to healthy children aged 6-59 months. Vaccine, 2009. 27(47): p. 6589-94.
25. Goff, P. H., et al., Synthetic Toll-like receptor 4 (TLR4) and TLR7 ligands as influenza virus vaccine adjuvants induce rapid, sustained, and broadly protective responses. J Virol, 2015. 89(6): p. 3221-35.

### Example 2: Generation of BECC438 and BECC470

For this example, we used BECC to exploit the normal bacterial LPS biosynthesis pathway present in all Gramnegative bacteria to synthesize the unique lipid A structure of our top candidates; BECC438 and BECC470 is shown in FIG. 2. Based on the ability of these molecules to induce a cytokine profile that is indicative of propagating protective immunity and in vivo data demonstrating the ability to successfully adjuvant known protective vaccine antigen (HA), we propose that BECC348 and BECC470 are excellent adjuvant candidate molecules for further development.

Characterization of BECC470. Novel BECC lipid A TLR4 molecules were made using BECC. This technique allows us to heterologously express specific enzymes (acyltransferase, deacylase, phosphatase and/or glycosyl-transferase) obtained from a wide variety of bacterial backgrounds to rapidly create unique lipid A-based structures allowing for the manipulation of the final immunostimulatory properties of the molecule. Ligand immunostimulatory properties were sequentially screened in vitro using HEK-Blue mTLR4, HEK-Blue hTLR4, THP-1, primary mouse splenocytes from both C57BL/6 and BALB/c backgrounds, primary human PBMC multiple donors, and human monocyte derived dendritic cells (DC). HEK and THP-1 cells were obtained from InvivoGen, mice from Jackson laboratories, and human primary cells from AllCells. BECC and the overall in vitro screening procedure are the topic of a manuscript from the co-P1 on this proposal, Dr. Ernst, and published in mBio¹⁷. The molecules that we present here, BECC438 and BECC470, and associated data were not published in the mBio manuscript. However, they have been screened in the same pipeline detailed in the paper.

The molecules presented in this example, BECC438 and BECC470, were generated using the attenuated Yersinia pestis (Yp), KIM6+ strain. This strain lacks the virulence plasmid pCD1 and contains a chromosomal deletion for the ping locus. Therefore, it is avirulent and exempt from NIH select agent guidelines (https://www.selectagents.gov/exclusions-hhs.html) and can be safely grown under biohazard safety level 2 conditions. This isolate of Yp has an identical lipid A structure as the Tier I Yp select agent stains, including C092. Specifically, BECC438 and BECC470 were engineered through the repair of the Yp PagP acyltransferase enzyme. The resulting hepta-acylated lipid A molecule is a hepta-acylated structure with three fatty acids (2-C16, 1-C12) attached to the four 3-OH C14 acyl groups on the diglucosamine backbone. Subsequently, LpxF, the 4′ position phosphatase from Francisel/a novicida, is expressed in BECC 470 resulting in the synthesis of a monophosphorylated lipid A. This is in contrast to MPL or PHAD, which has the 1 position phosphate removed. Similar to the GSK MPL, there is a small amount of heterogeneity in the lipid A structures present in any biologically made agent; the predicted structures represent the main molecule and were confirmed by mass spectometry and gas chromatography. When compared to hexa-acylated PHAD and E. coli lipid A structure (FIG. 2), these additional longer chain additions attenuate the pathogenic proinflammatory properties of the lipid A while maintaining adjuvanting immunostimulatory properties. This attenuation is thought to be due to alterations in binding and signaling through the TRL4 receptor complex.

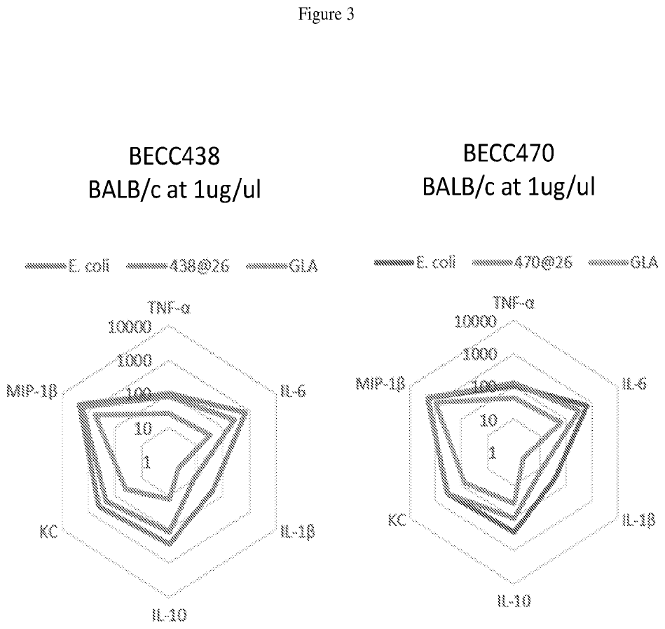

Once synthesized and highly purified to remove phospholipid and protein contamination, individual BECC molecules were screened using reporter cell lines for the ability to activate NFKB-driven innate immune responses. During this initial screen, BECC438 and BECC470 were identified as having strong adjuvant-like properities. They were capable of stimulating an innate immune response greater than PHAD, but less than the pyrogenic E. coli LPS. The initial description identified BECC438 and BECC470 as molecules that warranted further investigation including measuring the ability to initiate an immunogenic cytokine response in primary cell culture. When incubated with mouse primary splenocytes (BALB/c), as expected, it was found that BECC438 and BECC470 stimulated more cytokine release than PHAD but less than E. coli LPS (FIG. 3). It was particularly interesting that this differential cytokine production was observed in both the C57BL/6 (data not shown) and BALB/c mouse strains, which are known respectively to have a Th1 and Th2 immune response bias. The mouse splenocyte data provided further evidence that BECC438 and BECC470 are immunogenic across multiple genetic backgrounds, which is a necessary feature for any molecule proposed for use in the diverse human population.

To further characterize the immunogenic potential of BECC438 and BECC470, primary PBMC from three human donors were used and cytokine release measured by a Luminex Multiplex assay (FIG. 4). An expected heterogeneity in overall cytokine response profile was observed between donors, however BECC438 and BECC470 induced a cytokine response that is strikingly similar to that of PHAD. It is of particular importance that these molecule also induced less of the pyrogenic cytokine, IL-1β than E. coli LPS while still maintaining similar levels of immunogenic IL-10, IL-6, and MCP-1.

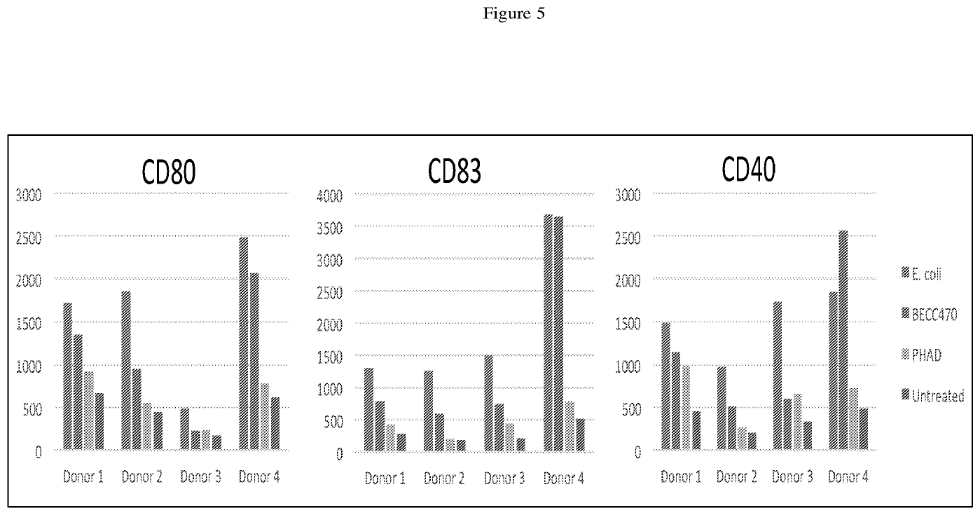

Costimulatory markers must be present on the membrane of APCs for a productive T-cell response to be initiated. Without costimulatory markers, T-cells will not recognize antigen. Finally, BECC470 was tested for the ability to stimulate upregulation of costimulatory markers on DCs, the APC population resident at site of vaccination, BECC438 analysis is in progress. Primary human monocyte derived DC from four separate donors were incubated with BECC470 and the presence of costimulatory molecules were measured by flow cytometry (FIG. 5). All DC assays were contracted to AIICells. We observe that the BECC molecules drove much higher costimulatory molecule surface expression than PHAD. This data, combined with the cytokine secretion data provides ample evidence that BECC438 and BECC470 will have attenuated reactogenecity while maintaining immunostimulatory properties.

Selection of Antigens and Model Systems. The objective of this proposal is to develop the adjuvant potential of BECC3438 and BECC470, both rationally designed and engineered TLR4-based ligands for evaluation as enhancers of an Influenza virus HA vaccine. BECC438 and BECC4 70 molecule will be evaluated for efficacy, immunogenicity, antigen sparing, and immune correlates of protection in adult and elderly mouse models when combined with HA. Both molecules will be studied in this proposal due to their potentially different responses in vivo. We feel it is critical to compare BECC molecules to standard adjuvants, Alum (alhydrogel) and PHAD in this work. These studies will allow development of these potential adjuvants through Influenza virus protection studies and compound optimization.

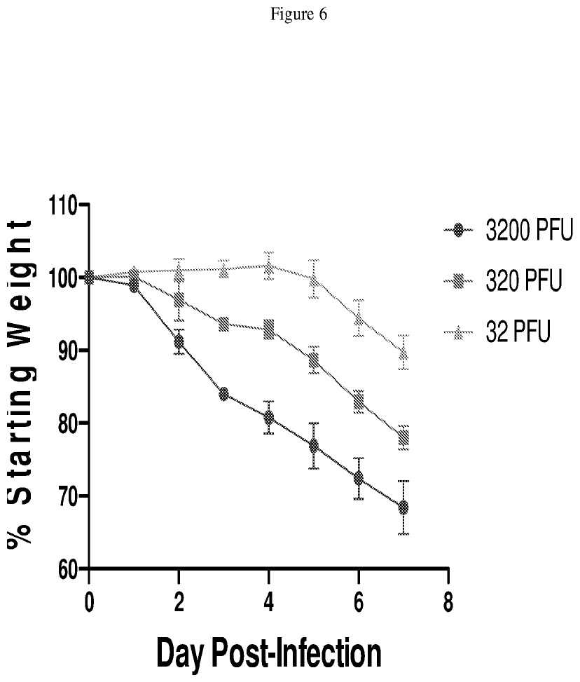

Influenza virus Challenge Model. Influenza virus is a human respiratory pathogen that has seasonal and pandemic potential. Seasonal infections occur around the world and are in response to the antigenic drift that occurs during replication of the virus in a host before it is transmitted to another host. The major antigenic determinants on the virus are the HA and NA proteins, with the HA being the dominant antigen targeted in current vaccine preparations. In 2009, a pandemic H1N1 Influenza virus (pH1N1) emerged, infecting an estimated 60.8 million cases (range: 43.3-89.3 million), 274,304 hospitalizations (195,086-402,719), and 12,469 deaths in the US alone (CDC). In the process of responding to the pandemic, it was determined that the planned vaccine in preparation in 2009 would not offer protection against the pH1N1 and a massive vaccine response was initiated to protect people across the world from the virus. The virus we will use in our challenge model is derived from the pH1N1 strain circulating in the Netherlands (strain A/Netherlands/602/2009, called NL/09 in this proposal (provided by Dr. Florian Kramer, Mt. Sinai Medical Center) and is identical to the A/California/04/2009 (Cal/09) strain circulating in the US. NUO9 causes significant weight loss, clinical symptoms and lung pathology in BALB/c mice (FIG. 6). Mice were intranasally inoculated with either 3200 pfu, 320 pfu or 32 pfu and their infection followed for 7 days. During this time, the mice inoculated with 3200 or 320 pfu lost >20% of their starting weight, had significant labored breathing and demonstrated significant viral load in their lungs. Due to the clinical, pathological, and viral growth phenotype, we will use 3200 pfu of NL/09 in our studies.

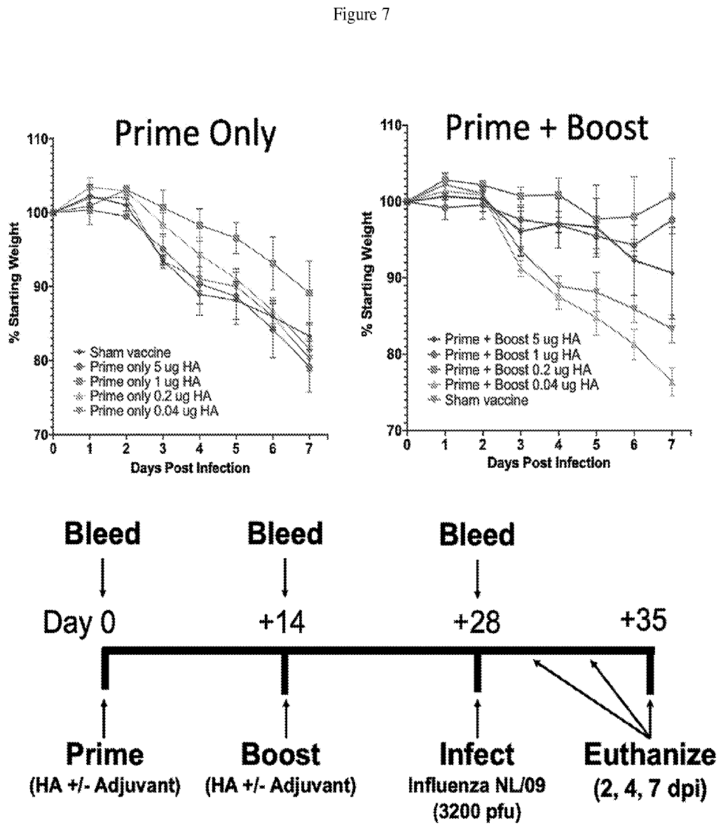

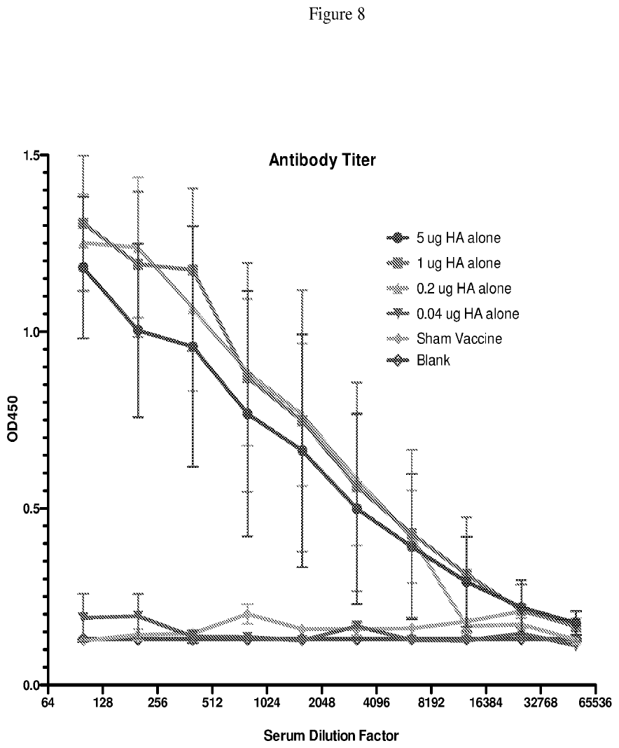

Influenza HA protein platform for vaccine: In our vaccination experiments, the HA protein, either alone or in combination with either Alum, PHAD, BECC348 or BECC470, is given as an intramuscular (i.m.) injection at 0 and 14 days of the vaccination timeline, with intranasal infection of the NL/09 or Sing/15 virus occurring at 28 days after the initial vaccination (FIG. 7). In preliminary experiments, the HA antigen was tested alone to determine the minimum non-protective dose for use in the studies. A dosing range of Sμg, 1 μg, 0.2 μg and 0.04 μg was used to initially evaluate the efficacy of protection by the HA protein alone. After either priming alone or a prime/boost timeline, we infected with mice with 320 pfu of NL/09 virus. We find that none of the prime alone vaccination groups were protected from weight loss and infection by the NL/09 virus. We do find that the 5 μg, 1 μg, 0.2 μg prime/boost dosing scheme does protect the mice from weight loss and viral infection and we find that the lowest 0.04 μg dose does not confer protection (FIG. 7). ELISA's were performed on sera from mice at day 28 before they were infected, and we find that the antibody titers corroborate with the protection data (FIG. 8). The prime alone groups demonstrate significantly lower antibody levels while the prime/boost groups are significantly higher. Importantly, the 0.04 g prime boost group does induce low level antibody but to unprotective levels. This identifies a lower limit of protection in this model where between the 0.2 μg dose and the 0.04 μg dose, protection is lost with protein alone.

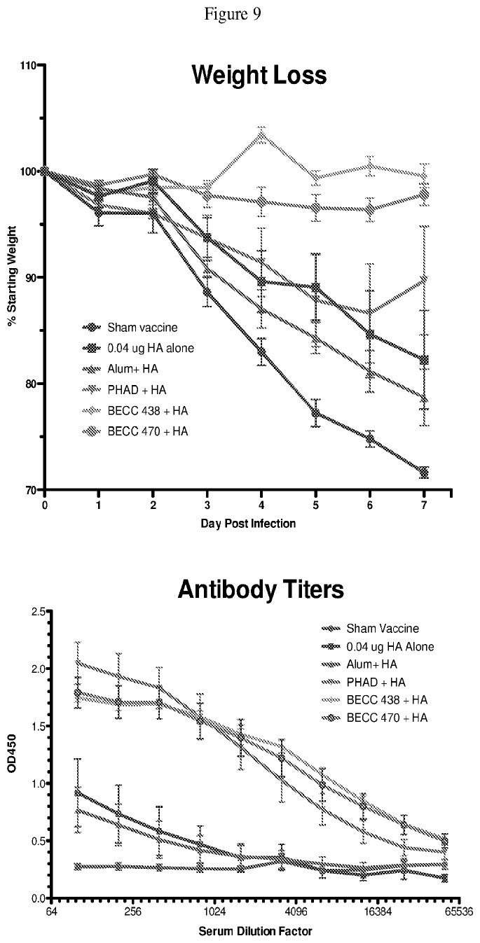

Adjuvanted HA vaccine with BECC438 and 470 protects mice from Influenza virus. We proceeded to test the HA protein at 0.04 μg with either Alum, PHAD, BECC438 or BECC470. At 0.04 μg, we find no protection from NL/09 infection by weight loss (FIG. 7) or virus titer (data not shown). In this experiment, all adjuvants were used at 50 μg per mouse. The identical prime alone or prime/boost strategy was used to determine if the BECC adjuvants conferred differential protection to Influenza virus challenge compared to the adjuvants Alum or PHAD. After challenge with 320 pfu of NL/09 by intranasal inoculation, we find significant differences in protection from clinical disease and weight loss between the groups (FIG. 9). The weight loss curves demonstrate that sham/HA, Alum/HA and PHAD/HA vaccinated groups had significant weight loss and clinical disease throughout the 7 days post infection. These groups lost −20% of their starting weight and had severe clinical symptoms including ruffled fur, hunched back and labored breathing. In comparison, the BECC348/HA and BECC470/HA groups showed only minimal weight loss and no signs of clinical disease. Prime alone for either of the groups showed no protection (data not shown). Antibody titers were evaluated on sera at day 28, from before the inoculation with virus. This ELISA data demonstrates that like the infection results, the sham/HA, Alum/HA and PHAD/HA vaccinated groups had significantly lower anti-HA antibody titer while the BECC348/HA and BECC470/HA groups demonstrated significantly higher antibody titer. The antibody data correlates directly with the level of protection seen via weight loss and clinical disease.

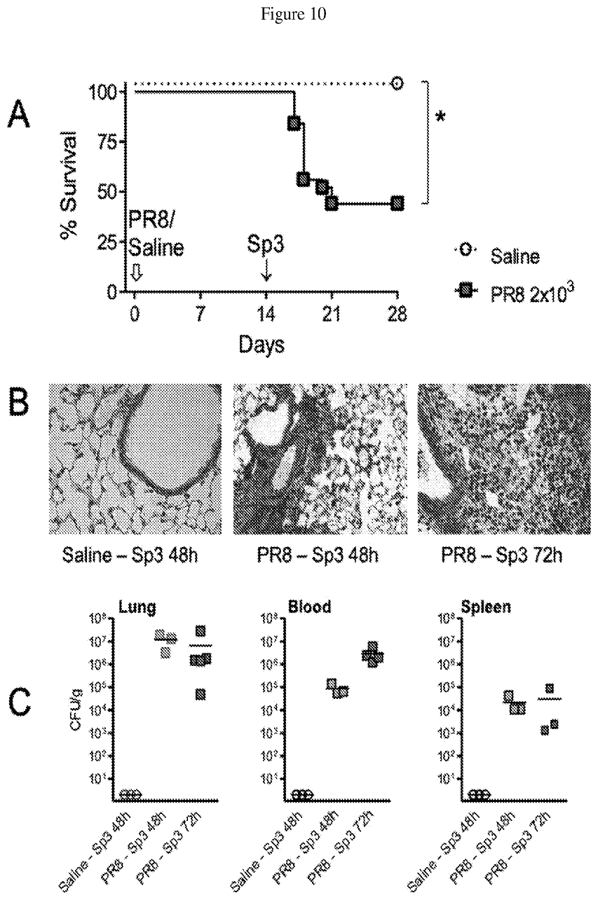

Sublethal secondary infection can become lethal after primary Influenza virus infection. The development of lethal disease in humans from Influenza virus infection is often due to a secondary bacterial infection rather than directly from the Influenza virus. We have previously modeled a secondary Streptococcus pneumoniae strain SP3 infection in mice either with or without prior Influenza infection (FIG. 10). We find that when given a normally sublethal dose of SP3, mice without previous Influenza infection show no weight loss, can control the bacterial load and have minimal inflammation. However, those mice that had been previously infected with Influenza virus have severe responses to the SP3, infection including substantial inflammation, increased CFU counts of the S. pneumoniae and increased death²⁶. Innate immune host defenses appear to be impaired following influenza, leading to susceptibility to subsequent bacterial infections via qPCR detection and analysis²⁶. We have shown that specific induction of IL-13 and IL-4 lead to alternatively activated macrophages (AAM) in the lungs that play a critical role in developing the hyper-susceptibility to secondary bacterial pneumonia. These alterations in lung development seem to be epigenetic and stable.

### Research Design and Methods

1: Formulate and evaluate dose sparing, adjuvant sparing, and cross protective immunity in an Influenza virus HA protein vaccine using BECC438 and BECC470 as an adjuvant in an Influenza virus model. Our preliminary experiments have shown that BECC438 and BECC470 are able to protect mice from Influenza virus infection when formulated with 0.04 μg of HA protein and 50 ug of each BECC adjuvant (formulated by vortexing BECC with antigen). In this aim, we will determine the minimal adjuvant and antigen capable of protecting mice from Influenza virus pathogenesis while producing significant anti-HA antibodies. We will also determine if BECC438 or BECC470 with HA produce protection against a heterologous challenge strain of Influenza, Sing/15.

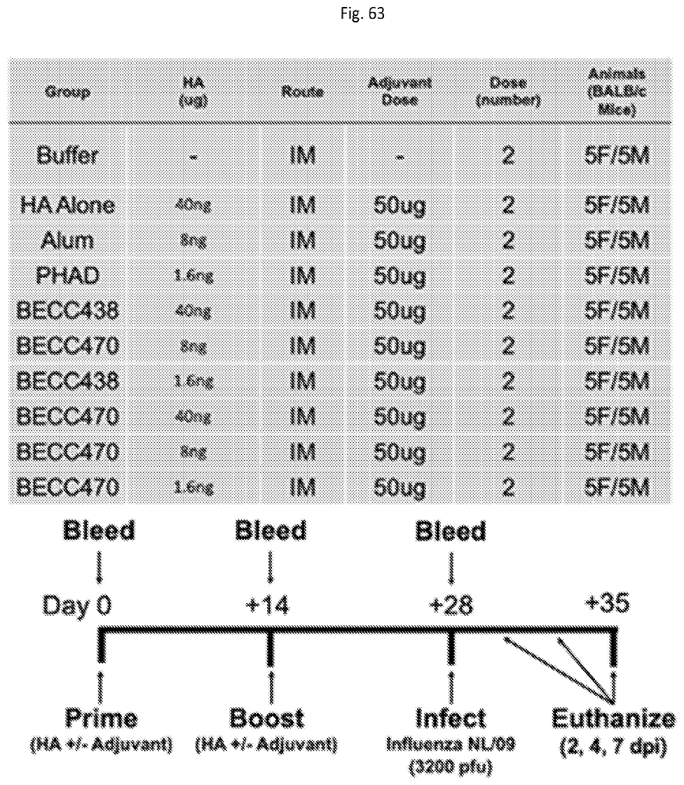

1A. Perform antigen-sparing experiments to identify the minimal amount of antigen needed for protection. In this subaim, formulations of the HA protein and TLR4 ligands BECC438 and BECC470 will be analyzed to determine the minimal amount of adjuvant required to achieve protection in an Influenza virus murine model. Formulations, as described previously¹⁷, will be tested for their immunogenicity and immune activation metrics when used in combination with the HA antigen (40 ng, 8 ng, and 1.6 ng) using a prime-boost administration as shown in FIG. 7. In this experiment, groups of eight 6-week-old female BALB/c mice will be vaccinated intramuscularly (IM) in the right caudal thigh with up to a 50 μL volume, depending on experiment according to the ten experimental groups described in FIG. 63. The vaccination and sample collection strategy are as follows: at day −2, all mice will be bled for pre-immune blood analysis (up to 200 μL of blood will be harvested from the right or left saphenous vein). At day 0, mice will be vaccinated with IM injections with the noted formulations. At day 14, mice will be bled and then boosted with IM injections. All mice will be bled 14 days later on day 28. On day 28, mice will be intranasally challenged with 320 pfu of Influenza virus NL/09. Mice will then be monitored for weight loss and clinical symptoms for the next seven days (day 28-35 from initial vaccination). Efficacy of vaccine will be evaluated as weight loss of challenged mice and immune correlates of protection will be measured in the pre-challenged sera by ELISA for HA specific antibody titers and neutralizing antibody titers by Influenza virus a hemagglutination assay. Additionally, mouse lungs will be harvested at day 2, 4 and 7 post infection for virus titering on MDCK cells and lungs will be fixed and stained with H&E to evaluate lung pathology. This experiment (n=10) will be repeated four times for a total of 40 mice per group, which will allow for statistical conclusions to be made about the efficacy of each adjuvant/formulation. From these experimental readouts, we will be able to determine the minimum amount of antigen necessary to induce high-level immunogenicity.

1B. Perform adjuvant sparing experiments to identify the minimal amount of adjuvant needed for protection. In this subaim, formulations of the HA protein and TLR4 ligands BECC438 and BECC470 will be analyzed to determine the minimal amount of adjuvant required to achieve protection in an Influenza virus murine model. Formulations will be tested for their immunogenicity and immune activation metrics when used in combination with the HA antigen (0.04 μg and the minimum dose identified in Aim 1A, shown in Table 1 as 1A dose) using a prime-boost administration as shown in FIG. 7. In this experiment, groups of ten 6-week-old female BALB/c mice will be vaccinated intramuscularly (IM) in the right caudal thigh with up to a 50 μL volume, depending on experiment according to the ⋅ten experimental groups described in Table 2. For this experiment, 1° F. and 10M mice will be used in each adjuvant dose group with 10 mice receiving the 40 ng HA vaccine dose used in our preliminary data and the other 10 mice dosed with the minimal protective HA vaccine dose identified in Aim 1A. If the 40 ng HA vaccine dose was shown to be the minimum effective dose in 1 A, it will then be used in this subaim. The vaccination and sample collection strategy will be identical to that described in Aim 1A. On day 28, mice will be intranasally challenged with 320 pfu of Influenza virus NL/09. Mice will then be monitored for weight loss and clinical symptoms for the next seven days (day 28-35 from initial vaccination). Efficacy of vaccine will be evaluated as weight loss of challenged mice and immune correlates of protection will be measured in the pre-challenged sera by ELISA for HA specific antibody titers and neutralizing antibody titers by Influenza virus a hemagglutination assay. Additionally, mouse lungs will be harvested at day 2, 4 and 7 post infection for virus titering on MOCK cells and lungs will be fixed and stained with H&E to evaluate lung pathology. This experiment (n=10) will be repeated four times for a total of 40 mice per group, which will allow for statistical conclusions to be made about the efficacy of each adjuvant/formulation. From these experimental readouts, we will be able to determine the minimum adjuvant combined with. minimal antigen necessary to induce immunogenicity.

TABLE 1
Adjuvant Sparing Study

| | | | | | | Animals |
|---|---|---|---|---|---|---|
| | HA | | Adjuvant | | Dose | (BALB/c |
| Group | (ug) | Route | Dose | | (number) | Mice) |
| Buffer Alone | — | IM | — | | 2 | 5F/5M |
| HA Alone | 40 ng/1A dose | IM | 50 | ug | 2 | 10F/10M |
| HA Alone | 40 ng/1A dose | IM | 5 | ug | 2 | 10F/10M |
| HA Alone | 40 ng/1A dose | IM | 0.5 | ug | 2 | 10F/10M |
| BECC438 | 40 ng/1A dose | IM | 50 | ug | 2 | 10F/10M |
| BECC438 | 40 ng/1A dose | IM | 5 | ug | 2 | 10F/10M |
| BECC438 | 40 ng/1A dose | IM | 0.5 | ug | 2 | 10F/10M |
| BECC470 | 40 ng/1A dose | IM | 50 | ug | 2 | 10F/10M |
| BECC470 | 40 ng/1A dose | IM | 5 | ug | 2 | 10F/10M |
| BECC470 | 40 ng/1A dose | IM | 0.5 | ug | 2 | 10F/10M |

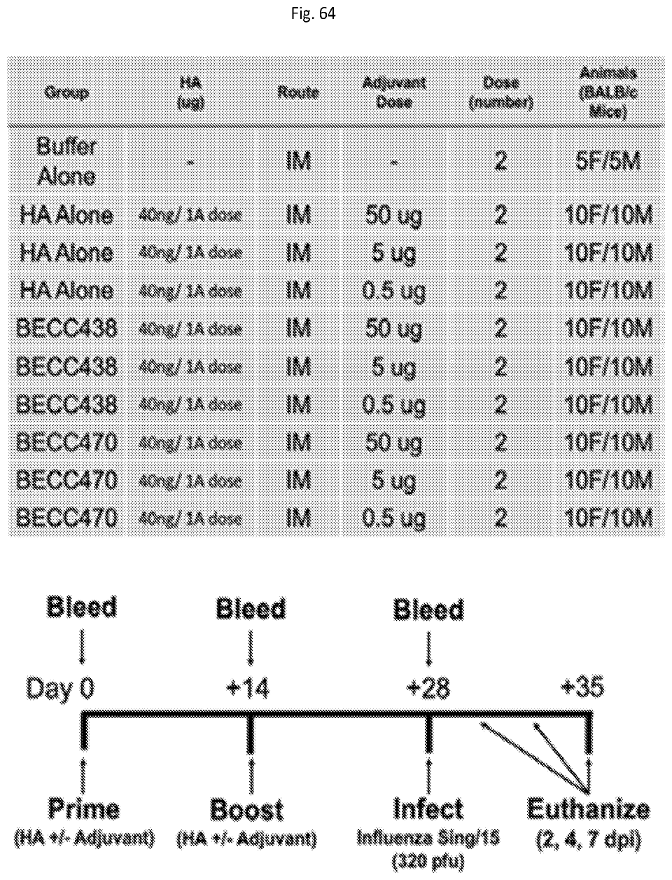

1C. Perform challenge experiments with a divergent Influenza virus (Sing/15) to determine if BECC/HA vaccination protects against heterologous challenge. In this subaim, formulations of the HA protein and TLR4 ligands BECC438 and BECC470 will be analyzed to determine if they confer cross protection against a divergent H1N1 Influenza virus strain, A/Singapore/GP1908/2015 (called Sing/15 in this proposal). The Sing/15 virus is a recombinant virus with the Influenza virus PR8 backbone segments with the HA segment containing the HA from the Sing/15 isolate, provided by Dr. Florian Kramer. It has been demonstrated that NL/09 does not cross protect against Sing/15 in mice (Dr. Florian Kramer, personal communication), however we hypothesize that the antigenic regions are potentially cross-reactive if antibody levels are of higher quantity and quality. The Sing/15 strain is highly pathogenic in BALB/c mice with preliminary data showing that the LD50 for the Sing/15 strain is 51pfu. Formulations will be tested for their immunogenicity and immune activation metrics when used in combination with the HA antigen using a prime-boost administration as shown in FIG. 7. We will use Cal/09 HA protein at 0.04 μg and the minimal dose found in Aim 1A in combination with either no adjuvant, Alum, PHAD, BECC438 or BECC470 adjuvants at 50 μg as shown in FIG. 64. The vaccination scheme will be identical to that in Aim 1A except challenge will be conducted with the Sing/15 strain at 102 pfu/mouse (2 times the LD50). Mice will then be monitored for weight loss and clinical symptoms for seven days post infection (day 28-35 from initial vaccination). Efficacy of vaccine to cross-protect from heterologous challenge will be evaluated as weight loss of challenged mice and immune correlates of protection will be measured in the pre-challenged sera by ELISA for HA specific antibody titers and neutralizing antibody titers by Influenza virus a hemagglutination assay. Additionally, mouse lungs will be harvested at day 7 post infection for virus titering on MDCK cells and lungs will be fixed and stained with H&E to evaluate lung pathology. This experiment (n=10) will be repeated four times for a total of 40 mice per group, which will allow for statistical conclusions to be made about the efficacy of each adjuvant/formulation. From these experimental readouts, we will be able to determine whether BECC adjuvants produce cross-reactive antibodies that protect against a heterologous challenge strain of Influenza virus. Should cross protection be found in this experiment, future experiments on dose and adjuvant reduction will be performed to compare required dosages of antigen and adjuvant for cross protection compared to homologous challenge.

Data analysis. We will compare the immunogenicity of the vaccines by comparison of the mean total IgG antibody levels (nonparametric Kruskal-Wallis 1-way analysis of variance with Dunn's multiple comparisons test for multiple groups or the Mann-Whitney U test) at day 14 (to assess speed of serum conversion), and at day 28 (final antibody levels after primary series). The primary consideration for selecting a vaccine for future study will be the ability to induce significant reduction mortality in murine intranasal Influenza virus challenge.

Rigor and reproducibility. In previous studies, when the challenge inoculum induces 25-30% weight loss (approved via UMB IACUC) with clinical morbidity, mice in the vaccine group provided protection in infection models at a p<0.01 level and this number of mice was sufficient to detect differences among different immunization regimens. We will be performing 4 independent experiments with 10 mice/group with equal number of males and females in each experiment and rigorous unbiased statistical analysis will be applied to compare outcomes within and between groups. The rigor and reproducibility rationale introduced here in Aim 1 and will be applied to all subsequent aims.

2: Determine efficacy of BECC438 and BECC470 adjuvanted Influenza vaccine in an immunosenescent/elderly mouse model of Influenza virus infection. This Aim will evaluate the protective capability of an adjuvanted Influenza HA vaccine in a lethal elderly Influenza virus model.

2A. Determine the lethal dose of NL/09 and Sing/15 in 12-month-old mice. We will determine the dosage of NL/09 virus that will lead to clinical disease, weight loss and potentially death in a 12-month-old mouse as a model of elderly immunosenescent individuals. The dosage determined will be used in Aim 2B for vaccination studies. We will intranasally inoculate 12-month-old BALB/c mice with the same strain used in the homologous challenge studies in Aim 1, (strain A/Netherlands/602/2009, called NL/09 in this proposal (provided by Dr. Florian Kramer, Mt. Sinai Medical Center). We have shown in FIG. 6, that NL/09 causes significant weight loss, clinical symptoms and lung pathology in 8-10-week-old BALB/c mice at either 320 or 3200 pfu/mouse. In Aim 2B, we will intranasally inoculate 12-month-old mice with either 3200, 320, 32 or 3.2 pfu/mouse of NL/09 virus (n=15 mice per group, 5 mice per timepoint). We will also will intranasally inoculate 12-month-old mice with either 500, 50, 5 or 1 pfu/mouse of Sing/15 virus (n=15 mice per group) for establishment of the dosing for future heterologous challenge experiments in Aim 2B. Lower doses of Sing/15 will be used due to its lower LD50 in adult BALB/c mice compared to NL/09. Mice will be observed for 7 days with weight and clinical score recorded for each mouse. Mice will be euthanized at day 2, 4 and 7 post infection (n=5 mice per timepoint) to analyze lung titer, viral RNA levels and lung pathology by H&E. The minimal dosage of each virus that causes weight loss, clinical disease and lung pathology will be used in Aim 2B for vaccine challenge studies.

2B. Determine the efficacy of vaccine formulations with escalating antigen dosing in 12-month-old mice. In this subaim, we will determine the amount of antigen needed to protect 12-month-oldmice. We hypothesize that higher amounts of antigen will be needed for protection of an agedmouse. Using a vaccination scheme similar to what is described in Aim 1, we will vaccinate and challenge mice with the adjuvants at the same concentration but with an increase in HA protein antigen.

HA protein will be combined with Alum, PHAD, BECC438, or BECC470 will be analyzed to determine the minimal amount of adjuvant required to achieve protection in an aged Influenza virus mouse model. Combinations will be tested for their immunogenicity and immune activation metrics when used in combination with the HA antigen (0.04 μg, 1 μg, 5 μg, and 25 μg) using a prime-boost administration as shown in FIG. 7. In this experiment, groups of ten 12-month-old male and female BALB/c mice will be vaccinated intramuscularly (IM) in the right caudal thigh with up to a 50 μL volume, according to the experimental groups described in Table 2. The vaccination and sample collection strategy are shown in FIG. 7 and are identical to Aim 1A. On day 28, mice will be intranasally challenged with the pfu dose identified in Aim 2A for the amount of Influenza virus NL/09 that is minimally lethal to the 12-month-old mice. Mice will then be monitored for weight loss and clinical symptoms for the next seven days (day 28-35 from initial vaccination). Efficacy of vaccine will be evaluated as weight loss of challenged mice and immune correlates of protection will be measured in the pre-challenged sera by ELISA for HA specific antibody titers and neutralizing antibody titers by Influenza virus a hemagglutination assay. Additionally, mouse lungs will be harvested at day 2, 4 and 7 post infection for virus titering on MDCK cells and lungs will be fixed and stained with H&E to evaluate lung pathology. This experiment (n=10) will be repeated four times for a total of 40 mice per adjuvant/HA group, which will allow for statistical conclusions to be made about the efficacy of each adjuvant/formulation. From these experimental readouts, we will be able to determine the minimum amount of antigen necessary to induce high-level immunogenicity.

TABLE 2
Vaccination with escalating doses of
HA protein + adjuvants in aged mice.

| | | | | | Animals |
|---|---|---|---|---|---|
| | HA | | Adjuvant | Dose | (BALB/c |
| Group | (ug) | Route | Dose | (number) | Mice) |
| Buffer | — | IM | — | 2 | 5F/5M |
| HA Alone | 0.04 ug, 1 ug, | IM | 50 ug | 2 | 40F/40M |
| | 5 ug, and 25ug | | | | |
| Alum | 0.04 ug, 1 ug, | IM | 50 ug | 2 | 40F/40M |
| | 5 ug, and 25 ug | | | | |
| PHAD | 0.04 ug, 1 ug, | IM | 50 ug | 2 | 40F/40M |
| | 5 ug, and 25 ug | | | | |
| BECC438 | 0.04 ug, 1 ug, | IM | 50 ug | 2 | 40F/40M |
| | 5 ug, and 25 ug | | | | |
| BECC470 | 0.04 ug, 1 ug, | IM | 50 ug | 2 | 40F/40M |
| | 5 ug, and 25 ug | | | | |

2C. Perform BECC/HA dose escalation experiment to determine an antigen:adjuvant combination that can protect with a single vaccination. In this set of experiments, we will determine if a single prime vaccination with an escalating dose of HA protein vaccine combined with adjuvant is able to protect mice from Influenza virus infection. A significant challenge in Influenza vaccination during a pandemic response is that often multiple vaccinations are required to respond to a novel Influenza virus strain. This was observed during the 2009 H1N1 Influenza virus outbreak where suboptimal antibody responses were seen across various formulations of the vaccine. We propose that with a highly immunologically active adjuvant that produces a balanced immune response, a single vaccination can achieve protection from Influenza virus challenge.

Similar to the vaccination in Aim 2B, HA protein will be combined with either Alum, PHAD, BECC438, or BECC470 will be analyzed to determine the amount of antigen/adjuvant combination required to protect mice from Influenza virus infection with a single dose of vaccine. Combinations will be tested for immunogenicity and immune activation when used in combination with the HA antigen (0.04 μg, 1 μg, 5 μg, and 25 μg) using a primeboost regimen, shown in FIG. 7. In this experiment, groups of 10 8-10-week-old male and female BALB/c mice will be vaccinated intramuscularly (IM) in the right caudal thigh with up to a 504 volume, according to the experimental groups described in Table 3.

TABLE 3
Vaccination with escalating doses of HA protein +
adjuvants in Prime only experiment.

| | | | | | Animals |
|---|---|---|---|---|---|
| | HA | | Adjuvant | Dose | (BALB/c |
| Group | (ug) | Route | Dose | (number) | Mice) |
| Suffer | — | IM | — | — | 5F/5M |
| HA Alone | 0.04 ug, 1 ug, | IM | 50 ug | 1 | 40F/40M |
| | 5 ug, and 25 ug | | | | |
| Alum | 0.04 ug, 1 ug, | IM | 50 ug | 1 | 40F/40M |
| | 5 ug, and 25 ug | | | | |
| PHAD | 0.04 ug, 1ug, | IM | 50 ug | 1 | 40F/40M |
| | 5 ug, and 25 ug | | | | |
| BECC438 | 0.04 ug, 1 ug, | IM | 50 ug | 1 | 40F/40M |
| | 5 ug, and 25 ug | | | | |
| BECC470 | 0.04 ug, 1 ug, | IM | 50 ug | 1 | 40F/40M |
| | 5 ug, and 25 ug | | | | |

Combinations will be tested for their immunogenicity and immune activation metrics when used in combination with the HA antigen (0.04 μg, 5 μg, and 25 μg) using a single prime administration as shown in FIG. 7 and challenged 28 days later. On day 28, mice will be intranasally challenged with 320pfu of Influenza virus NL/09.

Mice will then be monitored for weight loss and clinical symptoms for the next seven days (day 28-35 from initial vaccination). Efficacy of vaccine will be evaluated as weight loss of challenged mice and immune correlates of protection will be measured in the pre-challenged sera by ELISA for HA specific antibody titers and neutralizing antibody titers by Influenza virus a hemagglutination assay. This experiment (n=10) will be repeated four times for a total of 40 mice per group, which will allow for statistical conclusions to be made about the efficacy of each adjuvant/formulation. From these experimental readouts, we will be able to determine the minimum amount of antigen necessary to induce high-level immunogenicity.

3: Determine efficacy of BECC438 and BECC470 adjuvanted Influenza vaccine in a protecting mice from a sublethal secondary bacterial infection after Influenza virus infection followed by Streptococcus pneumonia (Sp) infection. This Aim will evaluate the protective capability of an adjuvanted Influenza HA vaccine in a secondary infection model. A severe bacterial infection is commonly associated with influenza and is a significant contributor to the excess morbidity and mortality of influenza. During interpandemic influenza, secondary bacterial infections may account for ˜0.25% of influenza-associated hospitalizations²⁷.

3A. Determination of sublethal and lethal Sp dosing in BALB/c mice. We will identify the sublethal and lethal doses of Streptococcus pneumoniae in BALB/c mice for use in the post vaccination and Influenza virus infection experiments in Aim 3B. The Streptococcus pneumoniae serotype 3 (Sp3) will be used due to its use as a model of secondary infection, especially in the lungs, and is the bacteria isolate that causes significant human disease in US patients.²⁷ The isolate (ATCC #6303) will be grown in Brain Heart Infusion Broth at 37° C. plus 5% CO₂ overnight, aliquoted into equal volumes of glycerol, and stored in −80° C. to be used for all studies. All strains required for this application are present in the Ernst laboratory stain bank.

We will use 6-8-week-old female, BALB/C mice (Jackson Laboratories, Bar Harbor, Me., USA) for the SP3 challenge experiments. Within one hour prior to challenge, frozen stocks of Sp3 will be diluted to the desired challenge concentration, in sterile, endotoxin-free phosphate buffered saline. Mice will be anesthetized with ketamine/xylazine prior to the deposition of 50 μl of challenge inoculum intranas ally (IN). In previous experiments, 5×10³ CFU of SP3 was shown to be sublethal in BALB/c mice. We will reconfirm this dosage by comparing 5×10², 1×10³, 5×10³ and 1×10⁴ CFU to identify which dosage provides the optimal sublethal dose. Each mouse will be followed daily for weight loss and clinical symptoms. Mice in groups at days 2, 4 and 7 post infection are will be harvested for lung, kidney and brain for CFU counts. We will choose the dose that results in minimal weight loss and clinical symptoms with no detected spread to other organs.

3B. Investigate if BECC438 and BECC470 plus HA antigen protects mice from sublethal Sp3 infection. We hypothesize that the lethality of Influenza virus infection followed by a normally sublethal secondary Streptococcus pneumoniae infection can be prevented by vaccination from the initial Influenza virus infection. We will determine whether an Influenza HA vaccine formulated with BECC438 or BECC470 is able to protect mice from a lethal secondary infection compared to other adjuvants. We will use the vaccination scheme in Table 6 where HA protein (0.04 μg) will be combined with Alum, PHAD, BECC438, or BECC470 (50 ug each). If lower amounts of HA protein and adjuvant are identified in Aim 1, those will be substituted for these concentrations in this experiment. with will be analyzed to determine the minimal amount of adjuvant required to achieve protection in an aged Influenza virus mouse model. Mice will be challenged with NL/09 after the primeboost regimen. At day 14 post NL/09 infection, mice will be challenged with S. pneumoniae SP3 at the dose identified in Subaim 3A. Mice will be weighted and scored for 7 additional days to follow the course of S. pneumoniae infection, as we have previously done for SARS-CoV and MERS-CoV mouse models.²⁸⁻³² In our previous experiments, ˜50% of mice that are challenged in this model without vaccination, succumb to infection within 4 days of S. pneumoniae challenge. At day 2 and 3 post S. pneumoniae infection, mice in each group will be euthanized and dissected for lungs, spleen and blood samples. Lungs and spleen will be fixed in 4% PFA for H&E staining for histology and with blood, CFU in each sample will be quantified as above. The remaining mice (n=10 per group) will be weighed and scored for clinical disease for a total of 14 days post S. pneumoniae infection to determine survival. This experiment will determine whether protection from Influenza virus leads to increased survival and decreased pathogenesis from a secondary S. pneumoniae challenge.

3C. Determine if alternatively activated macrophage induction is reduced in Influenza HA:BECC vaccinated mice. When the host is infected with influenza, an effective pulmonary response should result in sterilizing immunity while avoiding excessive bystander tissue damage and preventing “cytokine storm” to maintain oxygenation. The balance and regulation of inflammation during infection and the promotion of resolution during recovery from influenza remain underappreciated. However, alternatively activated macrophages (AAM) are increasingly recognized as important contributors to wound healing and dampening of pro-inflammatory responses as well as eliciting pathologic Th2 (allergy) responses²⁶. We will characterize the induction of AAMs before, during and after vaccination and challenge with SP3 infection. We have previously seen that in unvaccinated mice, after challenge with Influenza and then SP3, a large level of AAMs are induced²⁶. Lungs from Subaim 3B will be analyzed in this subaim for the levels of AAMs present in each stage of the infection. We will quantify the levels of IL-4 and IL-13, the key chemokines that drive AAM proliferation^{26,33}. We will also perform immunohistochemistry against FIZZ1, YM1, and ARG1, which are the key markers of AAM proliferation³³. We will compare unvaccinated and vaccinated mice after Influenza challenge and subsequent SP3 infection. We hypothesize that vaccination with BECC438 or BECC470 will quell the Influenza virus infection before lung damage occurs. Then once SP3 is inoculated, the bacterial infection will remain sublethal. Should the mice succumb to the infection, that suggests that protection from Influenza virus infection with a BECC:HA vaccine still allows for remodeling of the lung such that SP3 infection is lethal and potentially AAMs are still produced. Should the SP3 infection remain sublethal in the vaccinated mice, this will tell us that the strong immune response evoked by the BECC:HA vaccine is able to inhibit Influenza infection enough that minimal lung damage and immune alterations have occurred.

### LITERATURE (THIS EXAMPLE)

1. Krammer, F. et al. Influenza. Nat Rev Dis Primers 4, 3, doi:10.1038/s41572-018-0002-y (2018).
2. Palese, P. Influenza: old and new threats. Nat Med 10, 882-87, doi:10.1038/nm1141 (2004).
3. Wiwanitkit, V. Risk for worldwide pandemic of the new H7N9 influenza infection. J Biomed Res 27, 339, doi: 10. 7555/JBR.27 0.20130087 (2013).
4. Storms, A. D. et al. Worldwide transmission and seasonal variation of pandemic influenza A(H1 N1)2009 virus activity during the 2009-2010 pandemic. Influenza Other Respir Viruses 7, 1328-1335, doi:10.1 11 1/irv.12106 (2013).
5. Colizza, V., Barrat, A., Barthelemy, M., Valleron, A. J. & Vespignani, A. Modeling the worldwide spread of pandemic influenza: baseline case and containment interventions. PLoS medicine 4, e13, doi: 10.1371/journal.pmed.0040013 (2007).
6. Suarez, D. L. et al. Recombination resulting in virulence shift in avian influenza outbreak, Chile. Emerging infectious diseases 10, 693-699, doi:10.3201/eid1004.030396 (2004).
7. Treanor, J. Influenza vaccine—outmaneuvering antigenic shift and drift. The New England journal of medicine 350, 218-220, doi:10.1056/NEJMp038238 (2004).
8. Taubenberger, J. K. & Morens, D. M. 1918 Influenza: the mother of all pandemics. Emerging infectious diseases 12, 15-22, doi:10.3201/eid1201.050979 {2006).
9. Reid, A. H. & Taubenberger, J. K. The 1918 flu and other influenza pandemics: “over there” and back again. Laboratory investigation; a journal of technical methods and pathology 79, 95-101 11999).
10. Miller, M. S. & Palese, P. Peering into the crystal ball: influenza pandemics and vaccine efficacy. Cell 157, 294-299, doi:10.1016/j.cell.2014.03.023 (2014).
11. Surichan, S. et al. Development of influenza vaccine production capacity by the Government Pharmaceutical Organization of Thailand: addressing the threat of an influenza pandemic. Vaccine 29 Suppl 1, A29-33, doi: 10.1016/j. vaccine.2011.04.120 (2011).
12. Stephenson, I., Nicholson, K. G., Wood, J. M., Zambon, M. C. & Katz, J. M. Confronting the avian influenza threat: vaccine development for a potential pandemic. Lancet Infect Dis 4, 499-508, doi: 10.1016/S 1473-3099(04)01105-3 (2004).
13. Pittman, P. R. et al. Long-term duration of detectable neutralizing antibodies after administration of liveattenuated VEE vaccine and following booster vaccination with inactivated VEE vaccine. Vaccine 14, 337-343 (1996).
14. Tan, G. S. et al. Characterization of a broadly neutralizing monoclonal antibody that targets the fusion domain of group 2 influenza A virus hemagglutinin. J Viral 88, 13580-13592, doi:10.1128/JV1.02289-14 (2014).
15. Hiatt, A. et al. Glycan variants of a respiratory syncytial virus antibody with enhanced effector function and in vivo efficacy. Proc Natl Acad Sci USA 111, 5992-5997, doi:10.1073/pnas.1402458111 {2014).
16. Casella, C. R. & Mitchell, T. C. Putting endotoxin to work for us: monophosphoryl lipid A as a safe and effective vaccine adjuvant. Cellular and molecular life sciences: CMLS 65, 3231-3240, doi: 10.1007/s0001 8-008-8228-6 (2008).
17. Gregg, K. A. et al. Rationally Designed TLR4 Ligands for Vaccine Adjuvant Discovery. mBio 8, doi:10.1128/mBio.00492-17 {2017).
18. Gregg, K. A. et al. A lipid A-based TLR4 mimetic effectively adjuvants a Yersinia pestis rF-V1 subunit vaccine in a murine challenge model. Vaccine 36, 4023-4031, doi:10.1016/j.vaccine.2018.05.101 (2018).
19. Goff, P. H. et al. Synthetic Toll-Like Receptor 4 (TLR4) and TLR7 Ligands Work Additively via MyD88 To Induce Protective Antiviral Immunity in Mice. J Viro/ 91, doi:10.1128/JV1.01050-17 (2017).
20. Wirblich, C. et al. One-Health: A Safe, Efficient Dual-use Vaccine for Humans and Animals against MERS□CoV and Rabies Virus. J Viral, doi:10.1128/JV1.02040-16 (2016).
21. Coleman, C. M. et al. MERS-CoV spike nanoparticles protect mice from MERS-CoV infection. Vaccine 35, 1586-1589, doi:10.1016/j.vaccine.2017.02.012 (2017).
22. Taubenberger, J. K. & Kash, J. C. Influenza virus evolution, host adaptation, and pandemic formation. Cell host & microbe 7, 440-451, doi:10.1016/j.chom.2010.05.009 {2010).
23. Kilbourne, E. D. What are the prospects for a universal influenza vaccine? Nat Med 5, 1119-1120, doi:10.1038/13436 (1999).
24. Butler, D. The ghost of influenza past and the hunt for a universal vaccine. Nature 560, 158-160, doi:10.1038/d41586-018-05889-1 (2018).
25. Krammer, F. et al. A carboxy-terminal trimerization domain stabilizes conformational epitopes on the stalk domain of soluble recombinant hemagglutinin substrates. PloS one 7, e43603, doi:10.1371/journal.pone.0043603 (2012).
26. Chen, W. H. et al. Potential role for allernatively activated macrophages in the secondary bacterial infection during recovery from influenza. Immunology letters 141, 227-234, doi:10.1016/j.imlet.2011.10.009 (2012).
27. MacIntyre, C. R. et al. The role of pneumonia and secondary bacterial infection in fatal and serious outcomes of pandemic influenza a(H1N1)pdm09. BMC Infect Dis 18, 637, doi:10.1186/s12879-018-3548-0 (2018).
28. Frieman, M. B. et al. SARS-CoV pathogenesis is regulated by a STAT1 dependent but a type I, II and III interferon receptor independent mechanism. PLoS Pathog 6, e1000849, doi: 10.1371/journal.ppat.1000849 (2010).
29. Coleman, C. M. et al. Purified coronavirus spike protein nanoparticles induce coronavirus neutralizing antibodies in mice. Vaccine 32, 3169-3174, doi:10.1016/j.vaccine.2014.04.016 (2014).
30. Coleman, C. M., Matthews, K. L., Goicochea, L. & Frieman, M. B. Wild-type and innate immune-deficient mice are not susceptible to the Middle East respiratory syndrome coronavirus. The Journal of general virology 95, 408-412, doi:10.1099/vir.0.060640-0 (2014).
31. Pascal, K. E. et al. Pre- and postexposure efficacy of fully human antibodies against Spike protein in a novel humanized mouse model of MERS-CoV infection. Proc Natl Acad Sci USA 112, 8738-8743, doi:10.1073/pnas.1510830112 (2015).
32. Coleman, C. M. et al. CDS+ T Cells and Macrophages Regulate Pathogenesis in a Mouse Model of Middle East Respiratory Syndrome. J Virol 91, doi:10.1128/JV1.01825-16 (2017).
33. Page, C. et al. Induction of alternatively activated macrophages enhances pathogenesis during severe acute respiratory syndrome coronavirus infection. J Viral 86, 13334-13349, doi: 10.1128/JV1.01689-12 (2012).

### Example 3: BECC438 Successfully Adjuvants RSV F Protein Antigen

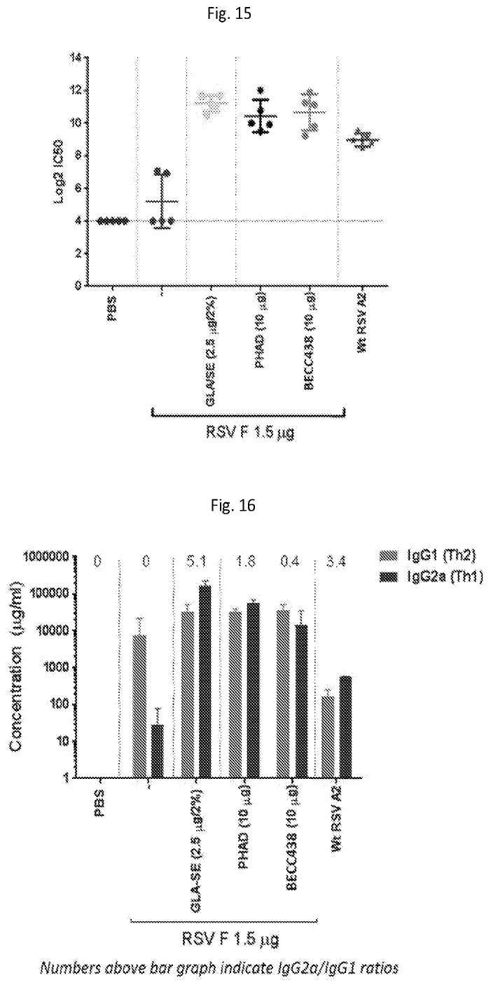

Our data demonstrate the ability of formulated BECC438 to adjuvant the post-fusion RSV F protein and provide protection to mice in an RSV challenge model. For all studies, the postfusion form of the protein was used at a concentration of 1.5 μg. Briefly, Balb/c mice were vaccinated intramuscularly (IM) on days 0 and 21 with PBS alone, RSV F alone, RSV F+GLA-SE (a reference TLR4 agonist formulated in an emulsion), RSV F+PHAD (a reference unformulated TLR4 agonist), RSV F+BECC438, then given a wild type RSV (A2) challenge. Serum was collected from mice on day 35 for measurement of RSV neutralizing antibody titers before infection, followed by challenged with RSV A2. Similar levels of neutralizing antibodies were detected in mice vaccinated with RSV F+GLA/SE, RSV F+PHAD, and RSV F+BECC438 (FIG. 15). These levels of antibodies are significantly greater than antibodies detected in the group that received RSV F protein alone, showing that PHAD and BECC438 both are able to boost immunity conveyed by the antigenic protein and are successful adjuvant molecules.

Antibody isotype was also quantified from the day 35 serum using an ELISA assay. It was found that mice vaccinated with RSV F+GLA/SE, RSV F+PHAD, and RSV F+BECC438 had relatively balanced IgG1 and IgG2a levels while mice that received the RSV F protein alone produced a much higher level of IgG1 than IgG2a (FIG. 16). These data indicate that all three adjuvanted vaccine solutions resulted in a more balanced Th1 vs. Th2 response than when mice are vaccinated with antigenic protein alone, with BECC438 initiating the most balanced response of the adjuvants tested.

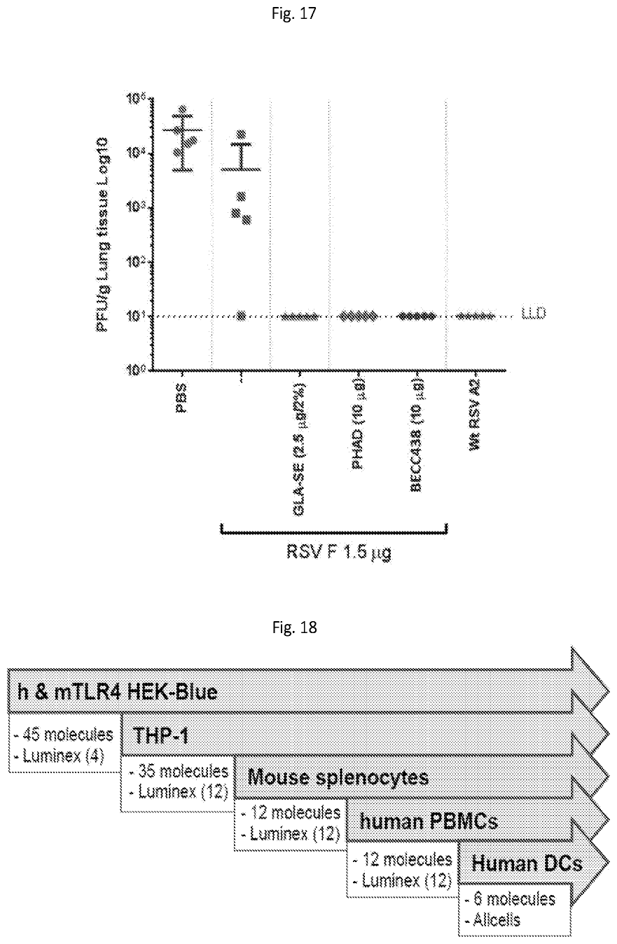

For quantification of viral titers, mice were euthanized four days after challenge (day 39) and tissue was collected as a measure of protection. Viral titers were measured in the lung and it was found that BECC438, as well as GLA-SE and PHAD, combined with RSV F protein were able to confer full protection in the lower respiratory tract (FIG. 17). Although these vaccines only have one antigenic virus protein, a similar level of protection is observed as when the mice have previously been exposed to a full live, replicating virus. The inability of the un-adjuvanted RSV-F protein to provide protection highlights both the necessity for an adjuvant, and the effectiveness of BECC438.

The data using an RSV F vaccination model are proof-of-principle for the efficacy of BECC438 as an adjuvant. In these initial studies, BECC438 was formulated into emulsions with the antigen before injection, although the dose of adjuvant used has not been optimized yet. The further RSV vaccine investigations proposed in this document will strive to develop an effective RSV vaccine adjuvanted with BECC438. Because a different formulation of RSV F protein, the antigen used in our preliminary data, has recently undergone vaccine clinical trials and shown to be not protective, we will use the significantly more antigenic form, the RSV pre-fusion F protein in our formulations. This antigen will be provided by Dr. Jason McLellan, Dartmouth University through a pay for server contract in sufficient quantities for all proposed experiments.

### Example 4: Characterization of BECC438

Novel lipid A TLR4 ligands were made using bacterial enzyme combinatorial chemistry (BECC). This technique allows us to heterologously express specific enzymes (acyltransferase, deacylases, phosphatase and/or glycosyl-transferases) obtained from a wide variety of bacterial backgrounds to create unique lipid A-based structures (TLR4 ligands) thus allowing rapid manipulation of the final immunostimulatory properties of the molecule. To date, over 70 novel ligands have been tested in vitro for the ability to initiate an immune response. Ligands were screened sequentially using HEK-Blue mTLR4, HEK-Blue hTLR4, THP-1; primary mouse splenocytes from both C57BL6 and Balb/c backgrounds, and multiple donors for primary human PBMC, and human monocyte derived dendritic cells (DC) (FIG. 18). BECC and this in vitro screening procedure are the topic of a manuscript from our lab recently published in mBio.

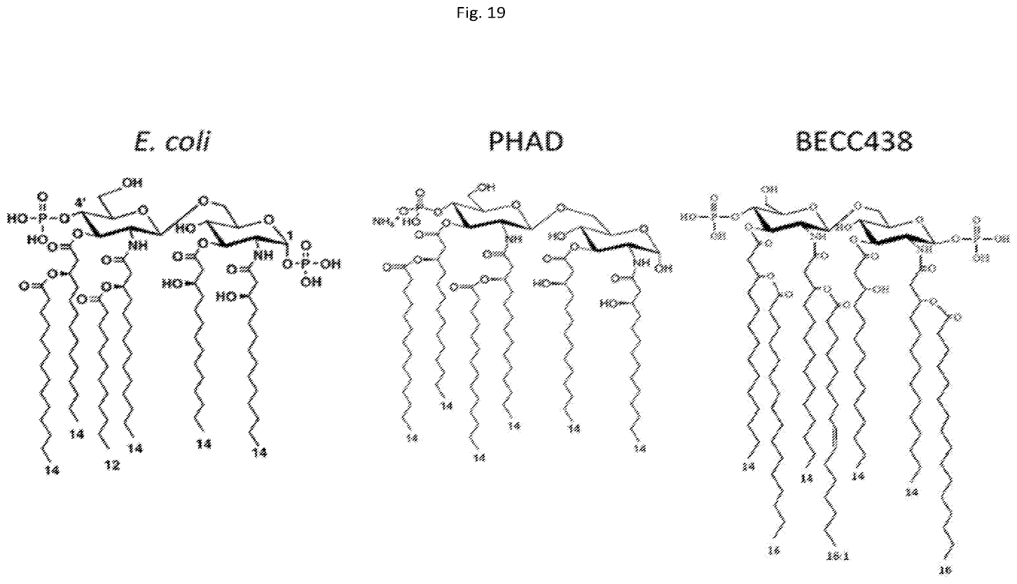

The molecule presented herein, BECC438 was generated using the attenuated Yersinia pestis (Yp), KIM6+ strain. This strain lacks the virulence plasmid pCD1 and contains a chromosomal deletion for the pmg locus. Therefore it is avirulent and exempt from NIH select agent guidelines (https://www.selectagents.gov/exclusions-hhs.html), and can be grown under biohazard safety level 2 (BSL2) conditions. This isolate of Yp has an identical lipid A structure as the Tier I Yp select agent stains, including C092. The Ernst laboratory is fully approved to work with all strains of virulent Yp under BSL-3 and aBSL-3 biosafety conditions (UMB IBC #0000133). Specifically, BECC438 was engineered through the deletion of the MsbB/LpxM enzyme, an acyltransferase for C14 addition, and repair of the PagP enzyme, a C16 acyltransferase. Mutation of the PagP enzymatic activity is one of the earliest known adaptations of Yp from Y. pseudotuberculosis (unpublished, Ernst laboratory) thereby blunting the host innate immune response. The resulting hepta-acylated lipid A molecule has three C16 additions, one with an unsaturation (C16:19) in additional to four 3-OH C14 fatty acids attached to the Y. pestis base lipid A (FIG. 19). Although there is a small amount of heterogeneity in the lipid A structures present of any biologically made agent, this predicted structure represents the main structure which was confirmed by mass spectometry. The structure has been further confirmed by gas chromotography. When compared to PHAD and E. coli lipid A (FIG. 19), this is a unique structure with C16 additions rather than C14 or C12. These longer chain additions attenuate the proinflammatory properties of the lipid A while maintaining immunostimulatory properties. This attenuation is thought to be due to inefficient binding and signaling through the MD-2 component of the TRL4 receptor complex. Once synthesized and purified, individual BECC molecules (TLR4 ligands) were screened using reporter cell lines for the ability to activate NFκB. During this initial screen, BECC438 was identified as having strong adjuvant-like properities. It was capable of stimulating an innate immune response greater than PHAD but less than the pyrogenic E. coli LPS. This initial description identified BECC438 as a molecule that warranted further investigation including measuring the ability to initiate an immunogenic cytokine response in primary cell culture. When incubated with mouse primary splenocytes (C57BL6 or BALBc), as expected, it was found that BECC438 stimulated more cytokine release than PHAD but less than E. coli LPS (FIG. 20). It was particularly interesting that this differential cytokine production was observed in both the C57BL6 and Balb/c mouse strains, which are known respectively to have a Th1 and Th2 immune response bias. The mouse splenocyte data provided evidence that BECC438 is immunogenic across multiple genetic backgrounds, which is a necessary feature for any molecule proposed for use in the diverse human population.

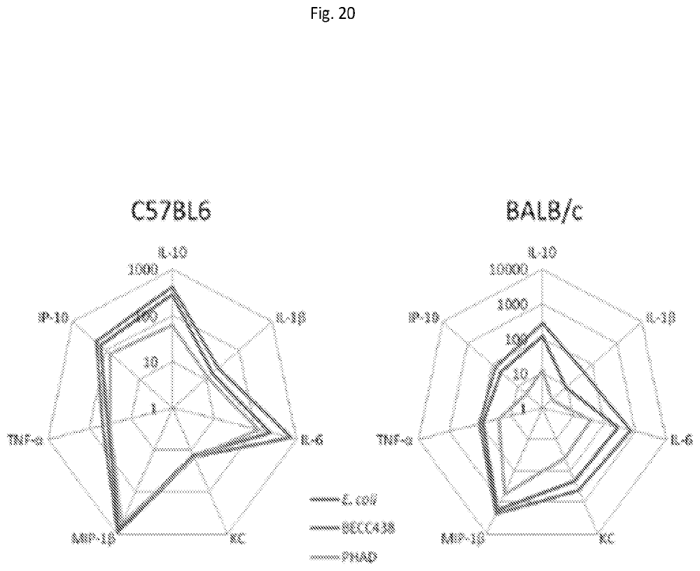

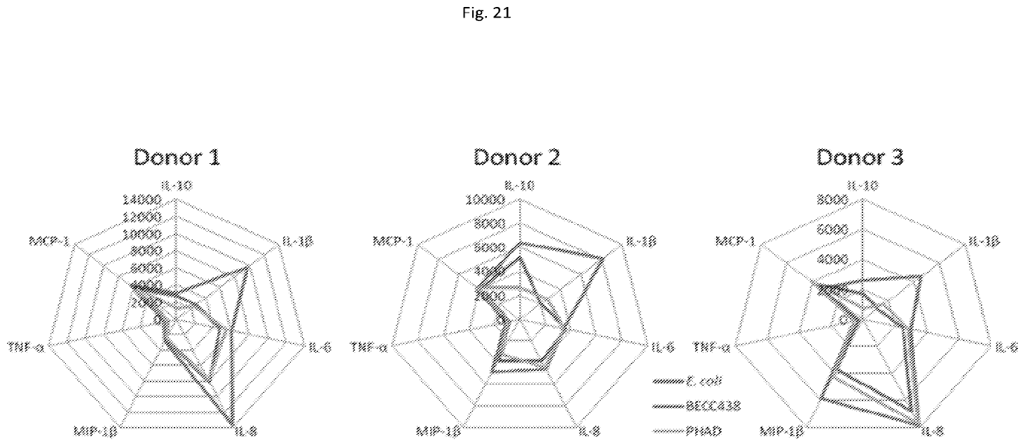

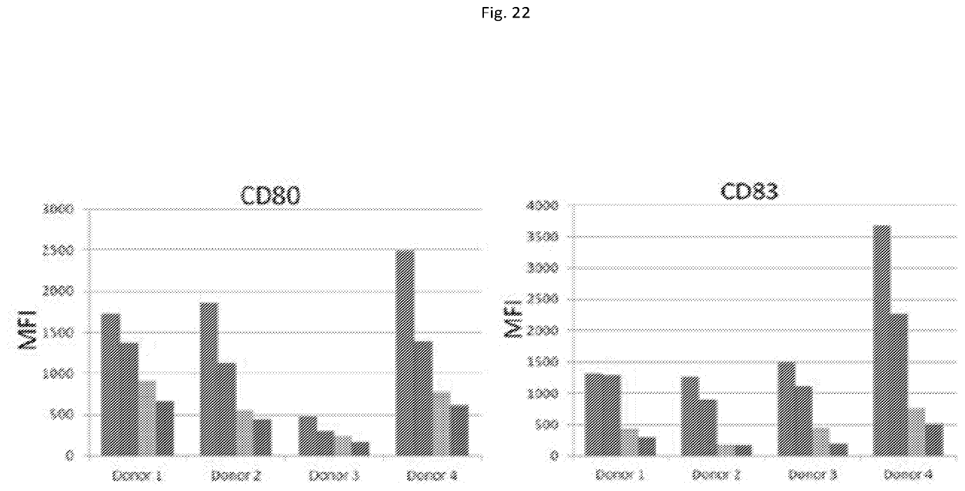

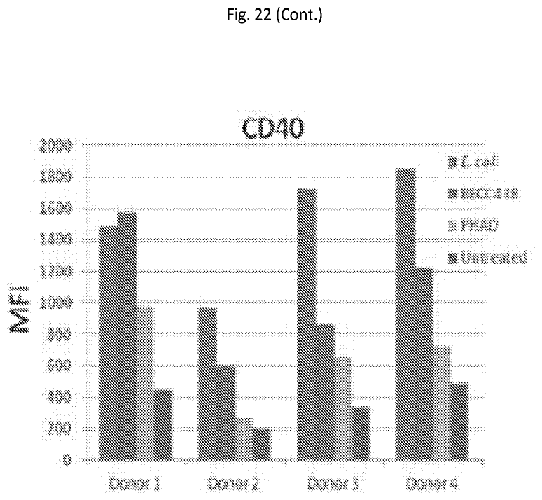

To further characterize the immunogenic potential of BECC438, primary PBMC from three human donors were used and cytokine release measured by a Luminex Multiplex assay (FIG. 21). Although an expected heterogeneity in overall cytokine response profile was observed between donors, BECC438 induced a cytokine response that is strikingly similar to that of the known adjuvant molecule PHAD. It is of particular importance that BECC438 induced less of the pyrogenic IL-1β than E. coli LPS while still maintaining similar levels of immunogenic IL-10, IL-6, and MCP-1. (FIG. 20). Activation primary mouse splenocytes by BECC438. Primary mouse splenocytes from C57BL6 and BALBc strains were incubated for 36 hours with 1000 ng/mL of E. coli LPS, BECC438, or PHAD. Cytokine secretion data, as measured by Luminex assay, is shown in pg/mL. 23 Costimulatory markers must be present on the membrane of antigen presenting cells (APC) for a productive T-cell response to be initiated. Without costimulatory markers, T-cells will not recognize antigen in the context of the need to respond. We tested the ability of BECC438 to stimulate upregulation of costimulatory markers on dendritic cells (DC), which are the resident APC population at site of vaccination. Primary human monocyte derived DCs from four separate donors were incubated with BECC438 and the presence of costimulatory molecules was measured by flow cytometry (FIG. 22). We observe that in most cases, BECC438 drove much higher costimulatory molecule surface expression than PHAD. This data, combined with the cytokine secretion data, provide ample evidence that BECC438 will have attenuated toxicity while maintaining immunostimulatory properties.

To create a synthetic version of the major structure of BECC438 (FIG. 19), we have established an NDA-covered relationship with Avanti Polar Lipids. Using their latest technology, they will be able to synthesize a pilot proof of concept lot (˜1 gm) of BECC438 (See Task 1 below). Additionally, they will have the ability to scale up to clinically relevant cGMP production (˜250 gms) as needed. This synthetic lipid, BECC438s, will undergo accelerated preliminary testing (as described below) and its efficacy will be compared to the biologic version, BECC438b. Although both molecules will have strong merit for use, once screening data is obtained, we will use defined criteria to decide to move into extensive vaccine studies with either BECC438b or BECC438s.

Formulation and Toxicity of BECC438

Formulation experiments have been completed and provide evidence that BECC438 can be successfully formulated into a vaccine and be injected into rodents without adverse reactions. BECC438 was formulated into four different systems, MEPC (4% Squalene, 1% DMPC (phosphatidylcholine)), MEPC-E (4% Squalene, 1% DMPC, 0.1% Vitamin E), MEPS (4% Squalene, 1% PS80 (Polysorbate 80)), or MEPS-E (4% Squalene, 1% PS80, 0.1% Vitamin E) using the RSV F-protein antigen in collaboration with MedImmune. Each formulation was sonicated at 50° C. and mixed in a high shear mixer with the aqueous phase (20 mM Histidine, 10% Sucrose, pH 6). This course emulsion was then microfluidized at 30,000 PSI for 10 passes to yield a nanoemulsion. This initial formulation experiment determined that BECC438 was successfully driven into the oil phase in both nanoemulsions containing Polysorbate 80. Similar formulation trials will have to be completed for each adjuvant/antigen combination to determine which combination of components results in the most complete delivery system (Task 2), thus allowing go/no-go decisions about which formulation to use in large scale studies.

During studies, BECC438 was injected unformulated into mice via intraperitoneally (IP) and intramuscularly (IM) dosing of up to 50 μg without observing signs of acute toxicity. We expect, similarly to MPL, that the effective dose of formulated BECC438 will be in the range of 0.5-10 μg per mouse. It is not anticipated that the effective formulated dose of BECC438 will have toxic effects though each vaccine antigen/adjuvant formulation must undergo extensive toxicity studies to move toward licensure. For this analysis, we are proposing to use the current gold standard rabbit model for full toxicity studies on each vaccine once it is properly formulated.

### Example 5: Optimization of RG1-VLP Vaccine Performance in Mice with Novel TLR4 Agonists

### Materials and Methods

BECC Compound Synthesis

The previously described BECC438 and BECC470 Yp KIM6+ strains (18-19) were grown in shaking culture at 26° C. for 18 h, within which time the culture reached an OD600 of 1.0-1.4. After pelleting bacteria from the liquid culture, lipooligosaccharide was extracted from the pellet as previously described using a double hot phenol method followed by three 2:1 (vol/vol) chloroform-methanol washes (18). Mass spectrometry was used to confirm the extracted lipid A structures (Bruker Microflex MALDI-TOF).

Physicochemical Analysis of BECC/Alum Formulations

The size distribution of Alhydrogel was determined using a Malvern Mastersizer 3000 (Malvern Instruments). First, 6 mL of dH₂O was added to the sample chamber and a background measurement was taken. 300 μL of Alhydrogel sample was injected for each measurement (Alhydrogel at 1 g/L) for a total light obscuration of ˜4%. A refractive index of 1.57 was used for Alhydrogel in the Mie scattering calculations for the determination of size. Size distributions were reported by number. Each sample is an average of ten instrumental replicates. Three independent sample measurements were performed.

Zeta potential measurements were performed using a Malvern Helix (Malvern Instruments). One mL of sample was placed in a plastic disposable capillary cell and zeta potential measured with a 632 nm laser in a 173° backscatter configuration. Each sample is an average of six instrumental replicates. The measurement was performed for three independent samples.

Dynamic light scattering (DLS) was performed using a Malvern Helix (Malvern Instruments). A 632 nm laser in a 173° backscatter configuration was used to measure autocorrelation functions. The method of cumulants was used to determine the average size. Custom aluminum cuvettes with quartz windows containing 50 μL of sample held at 25° C. were employed during the sample measurement. Three independent samples were analyzed with five instrumental replicates (10 s collection time) for each sample.

Vaccine Preparation

RG1-VLPs were manufactured by Paragon Bioservices and were combined with Alhydrogel (InvivoGen) at 40 μg/ml RG1-VLPs and 1 mg/ml Alhydrogel and incubated on a rocking platform for 1 h at 4° C. BECC438, BECC470, and PHAD (Avanti Polar Lipids) were solubilized in 1× Dulbecco's PBS then sonicated for 15 min and added to VLP/Alhydrogel formulations before 1 h 4° C. rocking. Gardasil-9 (Merck, recombinant 9-valent human papillomavirus vaccine) was used as a positive control comparator. After 1 h 4° C. rocking, immunizations were equilibrated to room temperature and administered to mice.

In Vivo Mouse Vaccination Studies

8-10-week-old female BALB/c mice (Jackson) were randomized into groups of 8-10 animals and immunized on days 0, 14, and 28 (2-week intervals) or 0, 21, 42 (3-week intervals). Mice were anesthetized with isoflurane before i.m. (intramuscular) injection into the quadriceps muscle with 50 μl dose volumes. In some cases, submandibular bleeds were performed on days 13/14 and 27/28 and terminal bleeds were conducted via cardiac puncture on isoflurane-anesthetized mice at day 42 or day 56. In some cases, spleens were also harvested on day 42. In order to measure longer-term Ab responses, some mice were not terminated on day 42 but were monitored with submandibular bleeds at days 42, 70, 98, and 125, before receiving a 4^{th} immunization on day 126, followed by terminal bleeds and spleen harvest 1 week later.

Mouse Blood Sera and Splenocyte Preparation

Terminal bleeds were collected in serum separator tubes (Fisher Scientific) at r.t. and centrifuged at 6000 g for 1.5 min. Cell-free sera was collected and stored at −80° C. Spleens and popliteal lymph nodes were dissociated using the GentleMACS Dissociator (Miltenyi). Cell suspensions were subsequently washed with HBSS (Gibco)+5% FBS (Gibco), lysed to remove red blood cells with ACK Lysing Buffer (Thermo-Fisher) and filtered through 70 μm strainers (BD Biosciences). Cells were counted with the Vi-Cell analyzer (Beckman Coulter) and resuspended in RPMI-1640 (Thermo-Fisher)+10% FBS.

HPV16-L1 and HPV16-L2 RG1 Epitope ELISAs

Mouse sera were subjected to quantitation by ELISA of antibodies specific to HPV16-L1 VLPs and to the HPV16-L2 RG1 epitope. For HPV16-L1 Ab quantitation, Maxisorp 96-well plates (Thomas Scientific) were coated with HPV16-L1 VLPs at 2.7 μg/ml in coating buffer (1×PBS+0.2% Proclin 300 (Sigma)) and used within 3-5 days after incubation at 4° C. ELISA plates were incubated with blocking buffer 1.5 h then washed 5 times with wash buffer using a BioTek EL405 plate washer. Sera samples were diluted in blocking buffer (4% skim milk, 0.2% Tween 20 in 1×PBS) at 1:2500 dilution and then serially diluted 1:2 on the plate for 7 more wells for a final volume of 100 μl/well.

Sera used for standards and positive controls were generated from mice vaccinated with RG1-VLPs+Alhydrogel, and BALB/c naïve mouse sera (Innovative Research) was used for negative controls. Incubation of sera samples was for 1 h at r.t., gently shaking (300 rpm), followed by plate washing. The secondary antibody conjugate goat anti-mouse IgG-horseradish peroxidase (HRP) (Sigma) was added to the plates at a dilution of 1:10,000 at a volume of 100 μl/well and plates were incubated again 1 h, r.t., gently shaking. After washing, freshly prepared TMB solution (KPL), according to manufacturer's instructions, was added at 100 μl/well and plates were incubated 25 min at r.t. protected from light. Reactions were stopped by the addition of 100 μl/well of 0.36N H₂SO₄. Plate optical density (OD) values were measured at 450/620 nm with a SpectraMax M5 (Molecular Devices) instrument and data processed by SoftMax Pro 6.3 (Molecular Devices). Antibody levels, expressed as ELISA units (EU/ml), were then calculated by interpolation of OD values from the standard curve by averaging the calculated concentrations from all dilutions which fell within the range of the standard curve.

For HPV16-L2 RG1 epitope-specific Ab quantitation, the ELISA procedure is virtually the same as for the L1 ELISA except for the use of NUNC streptavidin-coated 96-well plates (Thermo-Fisher) that were coated with 250 ng/ml N-terminal-biotinylated L2 peptide (L2 a.a. 17-36) (JPT) in coating buffer (0.1 M Tris buffer, 0.15 M NaCl, 0.1% Tween 20) at 100 μl/well. Plates were used after 1-day incubation at 4° C. Sera samples were diluted in blocking buffer 1:5,000 and then serially diluted 1:2. The secondary antibody conjugate was diluted 1:20,000 before being added to plates. All other procedural steps were as in the L1 ELISA protocol.

VLP and PsV Production

293-TTF cells (293T cells expressing a second large T antigen and furin provided by R. Roden) were plated at approximately 60-80% confluence in flasks and incubated at 37° C. for 24 h. Cells were co-transfected with codon-optimized HPV6-L1/L2 p6shell, HPV16-L1/L2 p16shell, HPV18-L1/L2 p18shell plasmids (kindly provided by J. Schiller, NCI, NIH), HPV39-L1/L2 p39Vitro plasmid (R. Roden), reporter plasmid pYSEAP (J. Schiller), or reporter plasmid Luciferase (AddGene) using Lipofectamine 2000 (Thermo Fisher) prepared in serum-free Opti-MEM media (Thermo Fisher). This transfection mixture was added to the cells which were incubated for 48 h and followed by trypsin-mediated cell harvest. Both cells and media were centrifuged, media was decanted, and cells resuspended in lysis buffer 1 (DPBS with 1 g/L D-glucose and 36 mg/L sodium pyruvate) for transfer to 1.5 mL siliconized tubes. Cells were pelleted and resuspended in lysis buffer 2 (DPBS+10 mM MgCl₂, 10% Brij58 (Sigma), and RNase A/T1 cocktail (Ambion)), then incubated for 48 h. Lysates were clarified by centrifugation at 10,000 g for 10 min at 4° C., then layered on top of an Optiprep (STEMCELL) gradient, underlaying 27%, 33%, and 39% Optiprep in a 5 mL thin-wall polyallomer tube. Gradients were ultracentrifuged using a SW55 Ti rotor (Beckman Coulter) at 50,000 rpm for 3.5 h at 16° C. with an acceleration of 5 and deceleration of 7. Nine fractions were collected by puncturing tube bottoms with a 18G needle. 500 μl were collected in tube 1 followed by 250 μl in 8 more tubes. Fraction volumes of 20 μl were electrophoresed on a Coomassie gel to assure purity, and fractions with substantial bands at the predetermined sizes were titrated on LoVo-T cells beginning at 1:125 dilution to assess alkaline phosphatase activity. All fractions that yielded a single band by Coomassie and also demonstrated high activity were pooled before use in fc-PBNA. The dilution factor for PsVs used in the neutralization assay was chosen by identifying the dilution that provided a signal 100-200-fold higher than background signal (no-virus negative control). All fractions that yielded single PsV bands by Coomassie and could demonstrate a signal 100-200 above background were then combined to make a final PsV pool for each HPV type.

Furin-Cleaved Pseudovirion-Based Neutralization Assay (fc-PBNA)

As described in Wang et al. (20), LoVo-T cells (ATCC CCL-229, human colorectal adenocarcinoma line) grown to 70-90% confluency were removed by Trypsin/EDTA treatment and seeded at 7500 cells/well in a 96-well flat-bottom plate and incubated for 24 h at 37° C., 5% CO₂. Pre-diluted (1:25) mouse sera samples were serially diluted 4-fold in DMEM+10% FBS media in another 96-well plate, including positive and negative control samples derived from RG1-VLP/Alhydrogel-vaccinated mice and naïve mice, respectively. Furin-cleaved pseudovirion (fc-PsV) particles (from HPV types 16, 18, 39, 6) were also diluted to pre-determined concentrations (1:1500 for HPV16/39, 1:500 for HPV6, 1:125 for HPV18 based on titration assays) and added to 96-well round-bottom plates followed by equal volume of serially diluted serum samples, and the plates were then incubated for 2 h at 37° C. After incubation, the serum/fcPsV particle mixtures were added to the 96-well flat-bottom plates previously seeded with LoVo T cells, and the plates were then incubated at 37° C. for 72 h, after which cell supernatants were transferred to 96-well Optiplates (Perkin-Elmer) and incubated at 70° C. for 45 min. Optiplates were then incubated on ice for 5 min and centrifuged briefly, before SEAP (secreted alkaline phosphatase) substrate (Caymen Chemical) was added, followed by 30 min incubation at r.t., protected from light. Plates were read on a SpectraMax M5 microplate reader. The PBNA titers are reported as the reciprocal of the dilution that caused a 50% reduction in SEAP activity in comparison to the fcPsV-infected cells without added sera.

### ELISPOT

Freshly isolated splenocytes were resuspended in culture medium (RPMI-1640+10% FBS) at 5e6/ml. The R&D Systems mouse IFN-γ ELISPOT kit was performed according to manufacturer's specifications. After blocking plates with culture media, stimulations were added to plates including 5 μg/ml HPV16-L1 VLPs, 0.5 μg/ml anti-CD3/anti-CD28 (BD Biosciences), and 1:2000 Cell Stimulation Cocktail (eBioscience) in volumes of 100 μl. Lastly, 2.5e5 splenocytes were added per well in triplicate in a volume of 50 μl and plates were incubated in a 37° C., 5% CO₂ incubator for 42-48 h. ELISPOT plates were then developed according to manufacturer's protocol. Plates were washed with the BioTek EL405 plate washer. After a developing step, plates were air-dried for 24 h and then imaged and spots counted on an ImmunoSpot Analyzer (C.T.L.) using ImmunoCapture software.

Flow Cytometry Analysis

Popliteal lymph nodes were harvested from mice 2 weeks post 3^{rd} immunization and processed by GentleMACS instrument. Cells were stained with LIVE-DEAD Fixable Viability Stain 510 (BD Biosciences) for cell viability and then blocked with Fc Block (BD Biosciences). Surface staining was conducted with a cocktail including CD4-PerCP-Cy5.5, B220-APC-Cy7, CXCR5-biotin, (all BD Biosciences) and PD-1-BV421 (BioLegend). Cells were washed and resuspended with APC-streptavidin (BD Biosciences), then permeabilized using the Mouse Foxp3 Buffer Set (BD Biosciences), followed by intracellular staining with Bcl-6-PE (BD Biosciences). Fluorescent signals were detected by flow cytometry using FACSCelesta instrument and FACSDiva (BD Biosciences), and gated populations were further analyzed with FCS Express software (De Novo). CD4+ B220− CXCR5+ PD-1+ cells were quantified as well as Bcl-6 expression of that population.

In Vitro Characterization of BECC Compounds

In vitro characterization of BECC470 was performed as previously described for BECC438 (19). Briefly, immortalized cells and ex vivo primary cells were cultured in the presence of BECC compounds or other known TLR4 agonists. Cell culture supernatants were measured for levels of cytokine secretion using a Milliplex MAP assay (Millipore). Human monocyte-derived dendritic cells were also cultured in the presence of BECC470 or TLR4 agonists and the resulting upregulation of surface co-stimulatory markers was measured by flow cytometry (AllCells).

Statistical Analysis

Statistical analyses were conducted with GraphPad Prism 7 software using one-way ANOVA nonparametric analysis with the Kruskal-Wallis multiple comparisons test. p<0.05 was considered significant.

### Results

BECC compounds tightly associate with aluminum hydroxide without disrupting particle size

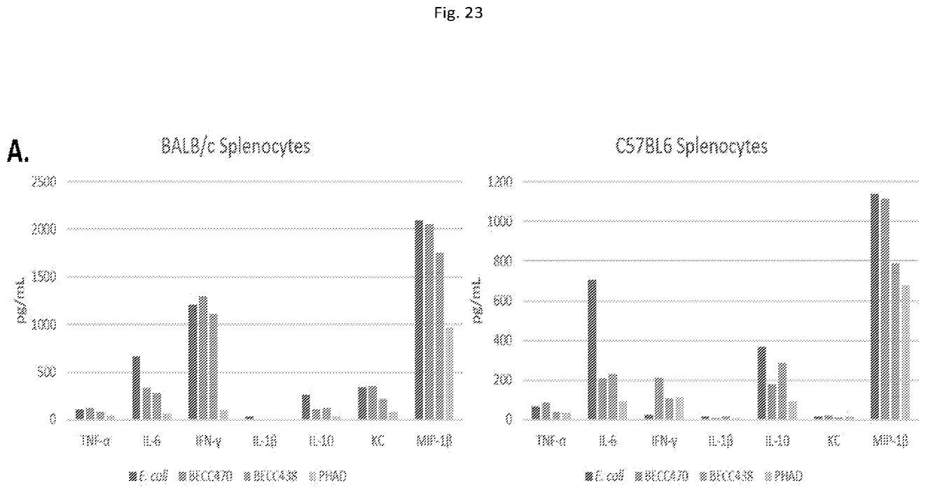

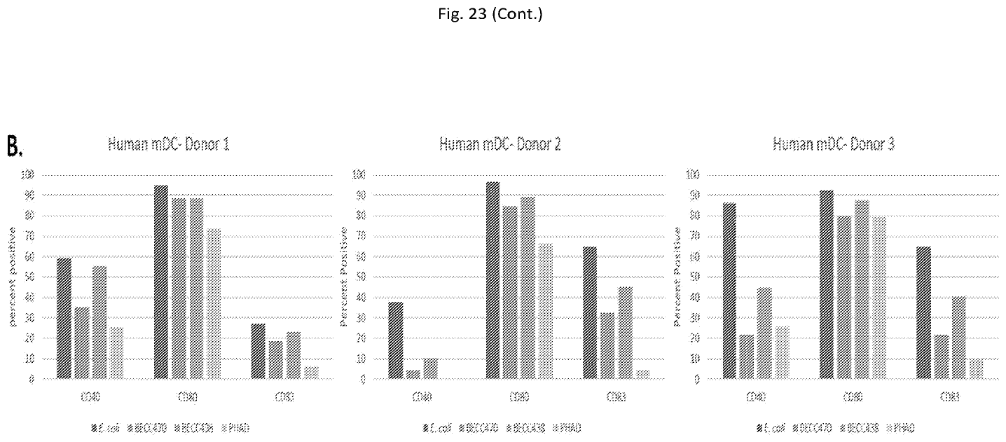

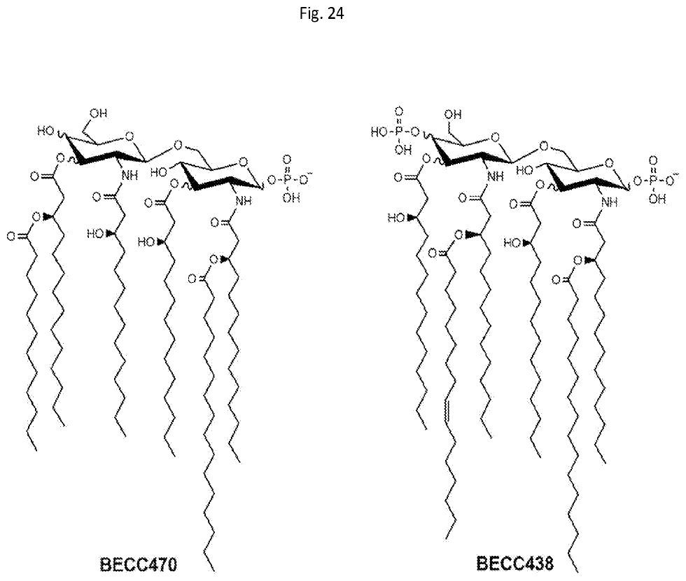

The compounds BEC438 and BECC470 were chosen for this study on the basis of robust in vitro activity profiles on HEK cell lines expressing human and mouse versions of TLR4 (FIG. 23) and demonstration of in vivo adjuvant activity in mice in the context of an antigen subunit-based vaccine with Alhydrogel (19). To quantify the interaction between BECC compounds and Alhydrogel, we constructed binding isotherms of BECC470 bound to 1 g/L and 0.2 g/L Alhydrogel. The isotherm at 0.2 g/L Alhydrogel reached a plateau at approximately 0.16 g/L BECC470 (FIG. 2A), indicating that Alhydrogel will become saturated with BECC470 at a 5:4 mass ratio under these solution conditions. Since the isotherm at 1 g/L Alhydrogel did not reach a plateau, a saturation mass ratio of 5:4 would predict a plateau at 0.8 g/L of bound BECC470. Using the slope of the first five points of each isotherm as a measure of binding affinity, 0.62 g and 0.95 g BECC470 is predicted to be bound per 1 g BECC470 added, respectively, for the 0.2 g/L and 1 g/L binding isotherms. Since ˜95% of the BECC470 is bound to Alhydrogel at the 1 g/L concentration, and a 10:1 mass of Alhydrogel:BECC470 is pharmaceutically relevant due to similarity to AS04 formulations (also a TLR4 agonist MPLA mixed with aluminum hydroxide), we chose a concentration of 1 g/L Alhydrogel and 0.1 g/L BECC470 to determine particle size distribution.

The size distribution of Alhydrogel in dH₂O spanned from 0.5 to 10 μm (FIG. 2B). The average size of Alhydrogel in dH₂O was found to be 1.05 μm, with a marginal increase to 1.66 μm for Alhydrogel in PBS and an intermediate size of 1.44 μm for Alhydrogel with BECC470 (Table 1). Alhydrogel is known to exchange with phosphate groups from other molecules which results in a general increase in negative charge. The zeta potential of Alhydrogel shifted from 12.3 mV in water to −13.7 mV in PBS. BECC470 formed particles with a diameter of ˜120 nm, similar to the 90 nm size reported for the aqueous formulation of a MPLA synthetic derivative glucopyranosyl lipid-A (GLA) (21). The BECC470 particles had a zeta potential of −7.9 mV, with the negative charge coming from the phosphate head-groups. When BECC470 was added to Alhydrogel in PBS, the zeta potential decreased further to −16.6 mV. These analyses demonstrate that BECC470 strongly associates with Alhydrogel particles in solution, has a negligible effect on alum particle size, and retains the negative charge of both molecules.

Enhanced Anti-L1/L2 Ab Levels and Cross-Neutralization Titers with BECC Compounds as Adjuvants

To investigate the adjuvant properties of Alhydrogel/BECC combination, we utilized RG1-VLPs, the novel chimeric VLP-based HPV vaccine which is comprised of 72 HPV16-L1 pentamers, each L1 subunit engineered to express a 20 a.a. sequence from the HPV16-L2 capsid protein termed RG1 (15). Unlike L1 capsid sequences, the RG1 epitope of the L2 sequence is well-conserved among disparate HPV strains and is known to provide a high-affinity B cell epitope for the generation of cross-neutralization antibodies in vivo in mice, and when adjuvanted with aluminum salt formulations such as Alhydrogel (15), is designed to provide protection to a broad repertoire of HPV strains. We immunized BALB/c mice intramuscularly three times with 2-week intervals between injections with RG1-VLPs and 50 μg Alhydrogel (alum) alone or with the synthetic TLR4 agonist PHAD, or the BECC compounds BECC438 and BECC470.

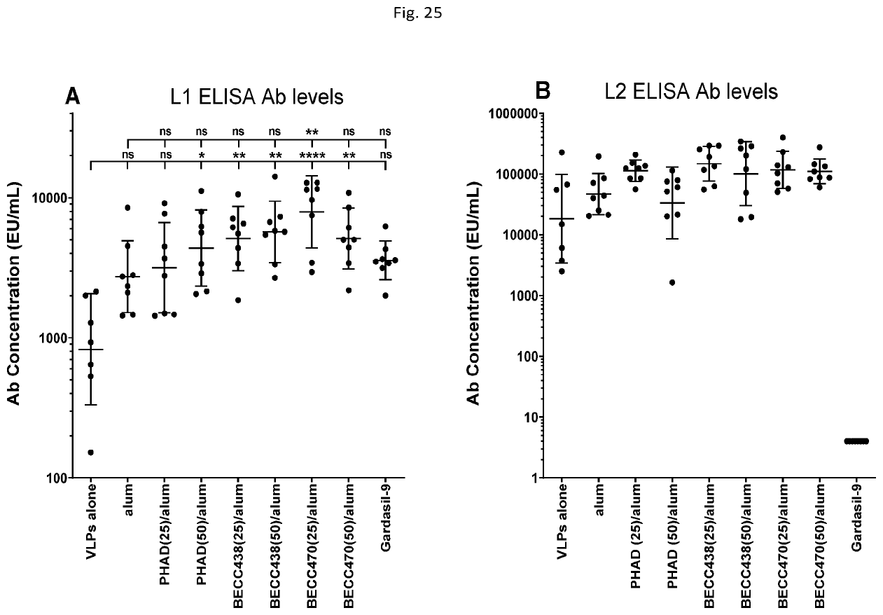

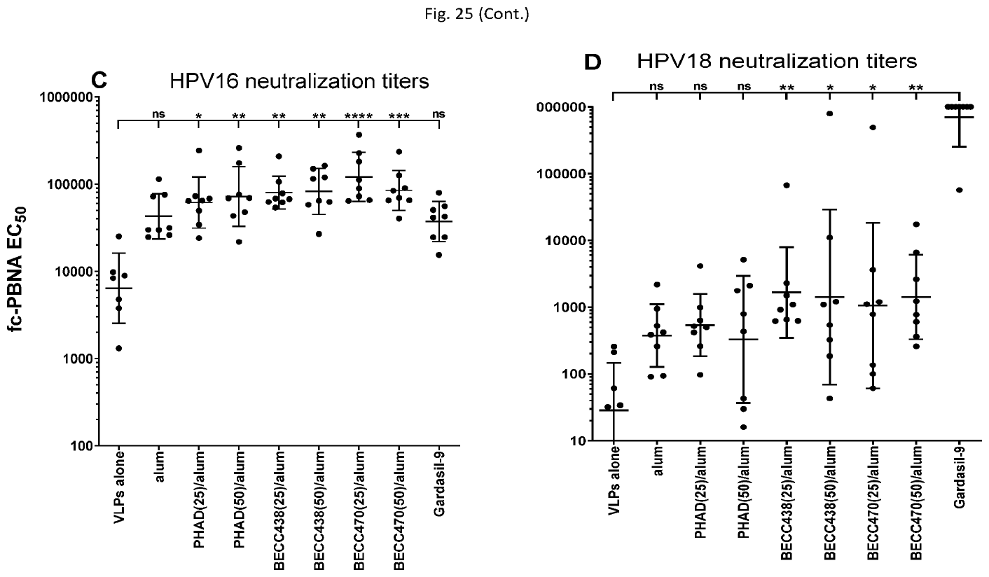

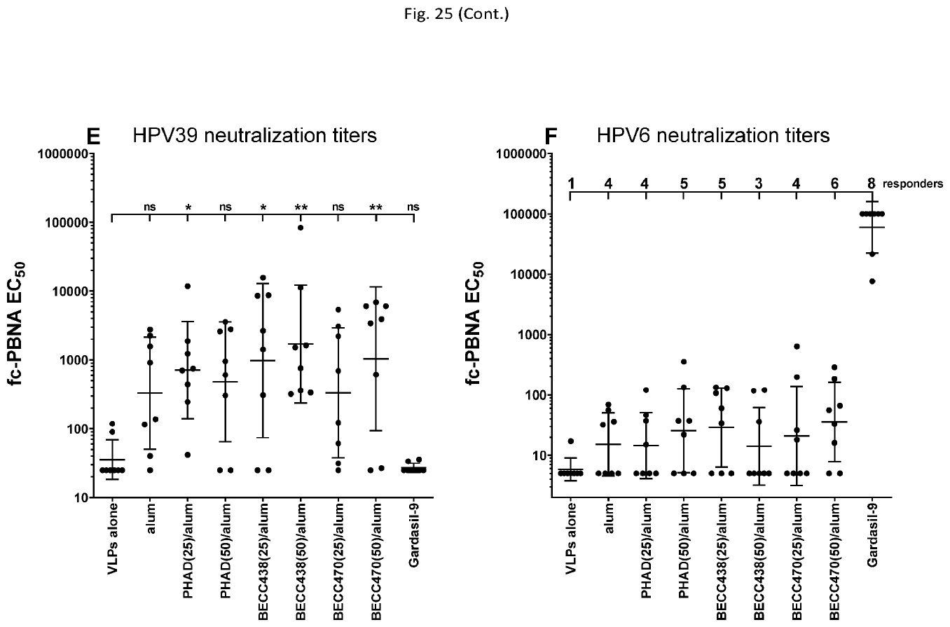

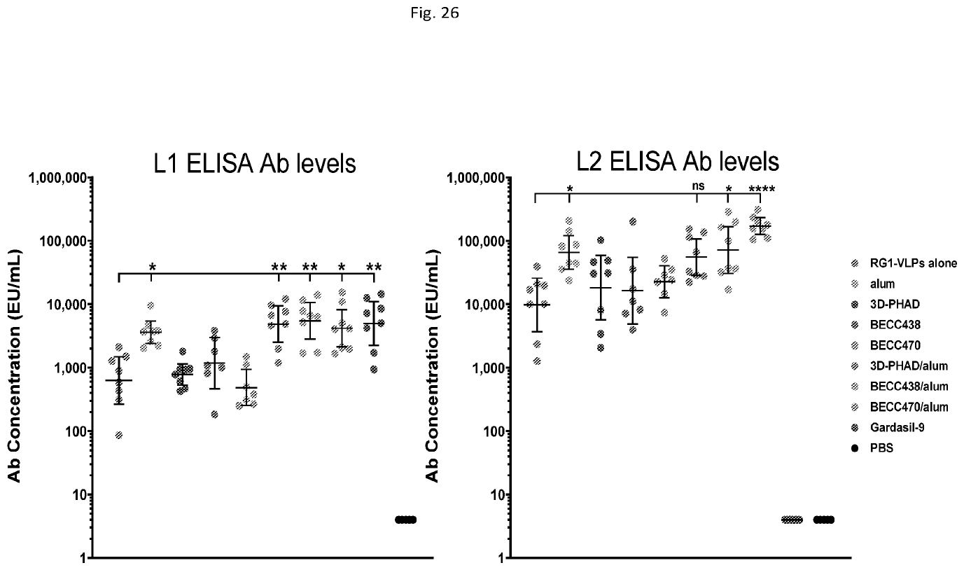

Supplementing alum with PHAD yielded greater L1 ELISA Ab levels compared to alum alone, but the additions of BECC438 and BECC470 achieved higher L1 geomeans and statistical differences over VLPs alone compared to PHAD, and BECC470 was the only agonist to achieve statistical relevance compared to VLPs+alum (FIG. 25A). Addition of BECC438 and BECC470 also elevated levels of Abs specific to the L2 RG1 epitope (FIG. 25B). BECC compounds utilized without an alum vehicle were substantially less active and did not significantly boost responses over VLPs alone (FIG. 26). An in vitro furin-dependent cell-based neutralization assay was developed to more closely resemble in vivo infectivity (22). Titers of neutralization Abs that prevent the in vitro infection of LoVo-T cells by SEAP-expressing HPV16 pseudovirions were substantially higher when the RG1-VLP vaccine had been adjuvanted by alum+BECC compounds and in particular by BECC470 (FIG. 25C). Cross-neutralization Abs elicited by the RG1-VLP vaccine were demonstrated by the presence of titers specific to HPV18 and HPV39, since the RG1-VLP is comprised of HPV16 L1 capsid subunits only. Adjuvantation with Alhydrogel raised the levels of HPV18- and HP39-neutralizing titers compared to VLPs alone, but the increase did not reach statistical significance (FIG. 25D-E). However, the presence of BECC compounds further increased the titers of both Ab species and achieved statistical relevance. Finally, cross-neutralizing titers to HPV6 proved the most difficult HPV substrain for TLR4L (TLR4 ligand)-augmented Alhydrogel to generate substantial boosting of responses (FIG. 25F).

Optimization of RG1-VLP Vaccine with BECC470 Allows for VLP Dose-Sparing and Decreased Number of Immunizations

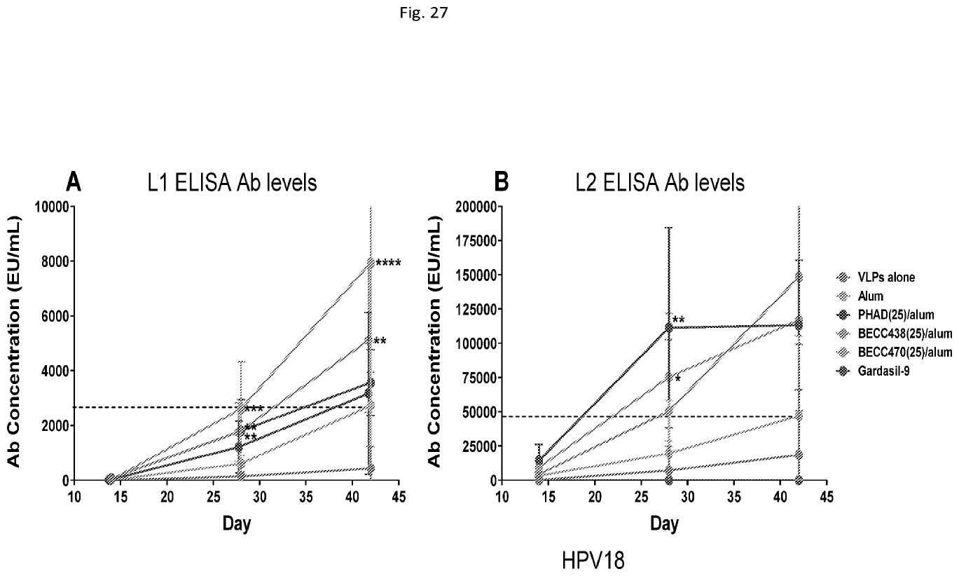

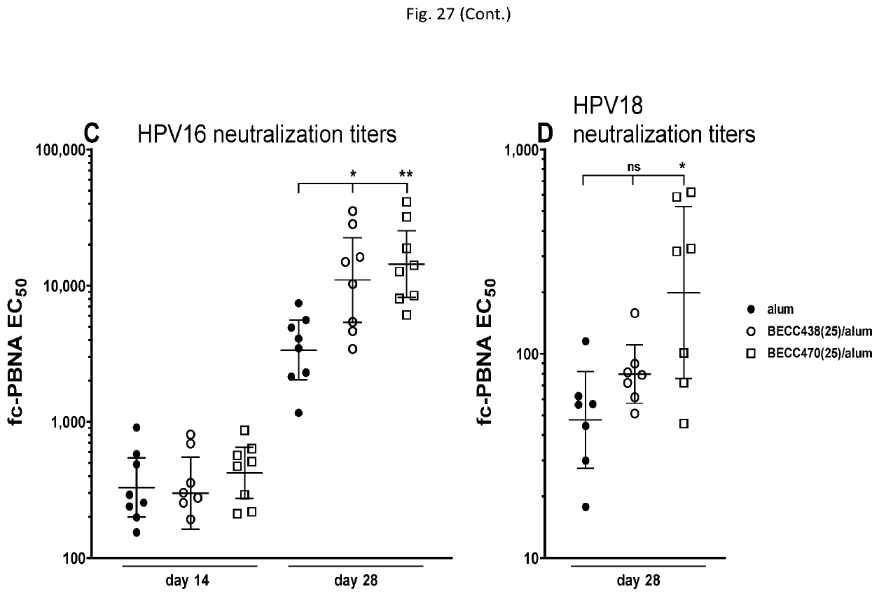

The RG1-VLP vaccine requires two additional boosts after the initial prime vaccination to yield substantial levels of L1 and L2-specific Abs when adjuvanted with Alhydrogel alone (14). To determine whether the addition of BECC438 and BECC470 could accelerate the kinetics of the humoral response to RG1-VLP vaccine, we measured Ab levels to both L1 and L2 at earlier timepoints, 2 weeks after first immunization (day 14) and 2 weeks after the second immunization (day 28) as well as 2 weeks after the third immunization (day 42). Both BECC438 and BECC470 adjuvants substantially elevated L1 and L2 ELISA Ab levels over alum alone after only 2 immunizations (FIG. 27A-B). Additionally, the presence of BECC470 enabled the RG1-VLP vaccine with 2 vaccinations to achieve L1 and L2 Ab levels equivalent to or superior to L1 and L2 Ab levels achieved by 3 vaccinations with alum alone. BECC470 also accelerated the appearance of neutralizing Ab titers to HPV16/18 PsVs, achieving superiority to alum alone by day 28 (FIG. 27C-D). These data emphasize the potential of a combined alum/BECC compound adjuvant allowing for fewer booster shots of the RG1-VLP vaccine.

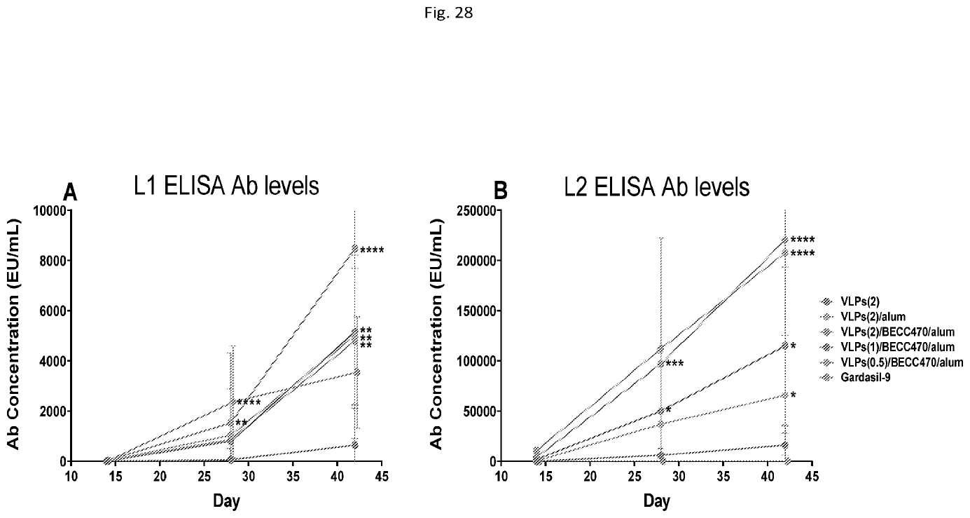

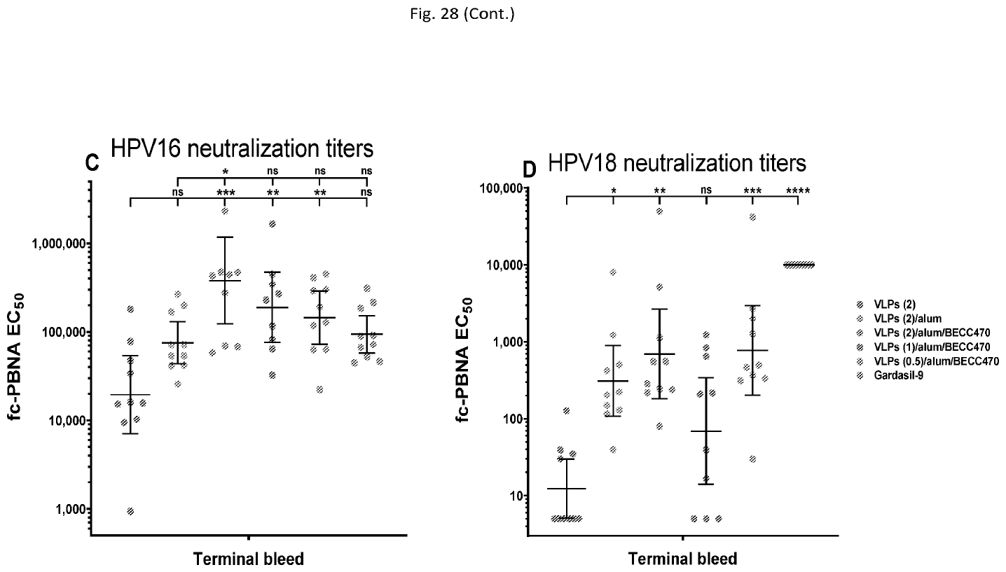

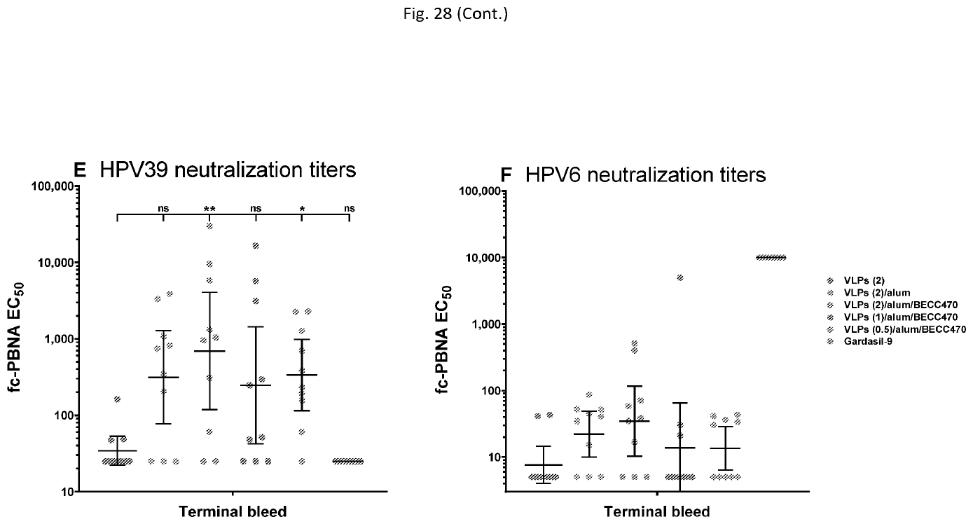

As the manufacture of various strains of VLPs is one of the main components of the high cost of commercial HPV vaccines Cervarix and Gardasil, VLP dose reduction would increase cost-effectiveness and might be achieved with proper adjuvant formulation. We investigated whether adjuvanting with BECC470/alum would allow dose-sparing of the VLPs in the murine vaccine model. (FIG. 28A-B) indicates that dropping the RG1-VLP dose to 1 μg or even 0.5 μg while adjuvanted with BECC470/alum still resulted in L1 and L2 ELISA Ab levels that were comparable (L1) or superior (L2) to levels achieved by alum alone with the full 2 μg VLP dose, indicating that BECC470 does allow for VLP dose-sparing. Neutralization assays run on the same sera samples revealed that induction of HPV16, HPV18, HPV39, but not HPV6, neutralization titers by 0.5 μg RG1-VLPs+BECC470/alum was also comparable to titers achieved by 2 μg RG1-VLPs+alum alone (FIG. 28C-F). Such robust humoral responses arising from only 25% of the standard mouse VLP dose underlines the benefits in dose-sparing and COG-savings that could be achieved with an appropriately potent adjuvant combination.

BECC470 Enables Long-Lasting Enhanced Ab Responses as Well as Robust Memory L1-Specific T Cell Responses

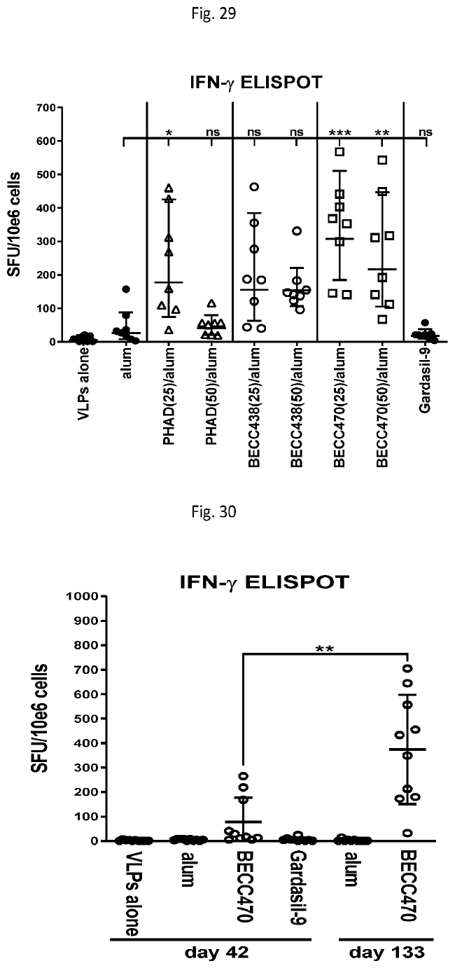

Although it is clear that neutralizing Abs that inhibit HPV infection are critical for a protective response, it is less clear what role HPV-specific T cell responses may play. Alum-formulated vaccines are historically poor for induction of T cell responses due to the activation of the inflammasome pathway which does not optimally prime DCs to activate T cells. Combining either BECC compound with alum in our vaccine led to robust HPV16-L1 VLP-specific T cell responses as measured by ELISPOT (FIG. 29), while alum alone was typically poor. An additional boosting effect on this T cell response could be observed in mice that received a 3rd boost approximately 3 months after the 2nd boost (on day 42) (FIG. 30), indicating the presence of long-term memory T cells. Weaponizing this additional arm of the immune response could lead to higher vaccine efficacy by developing a cytotoxic T cell response specific for HPV-infected cells.

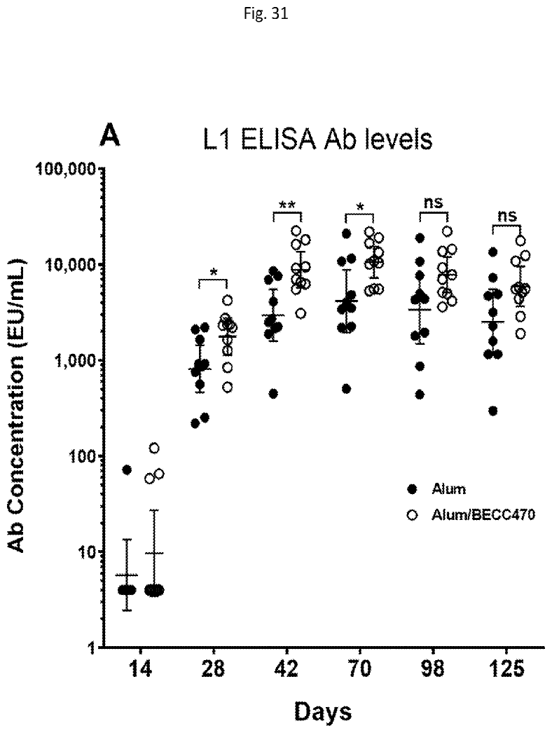

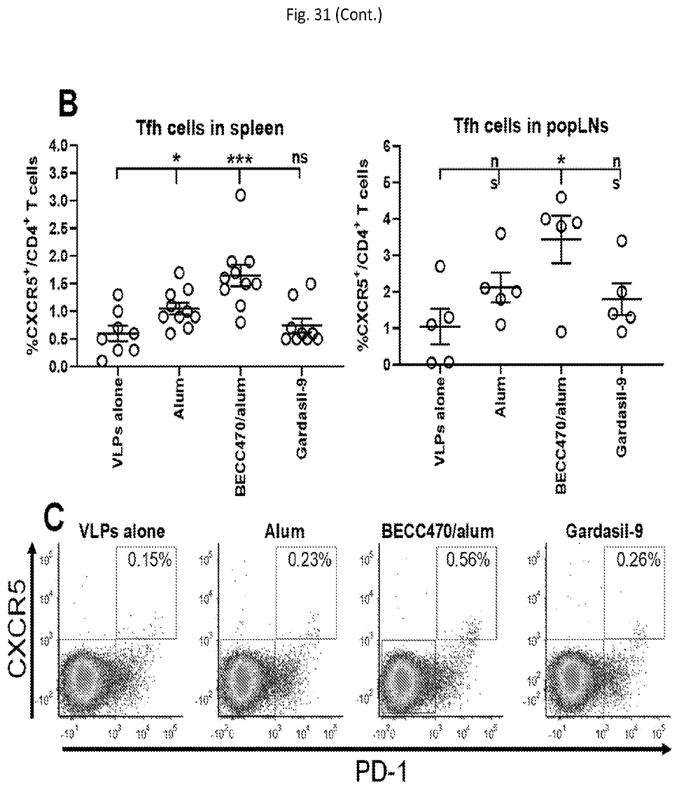

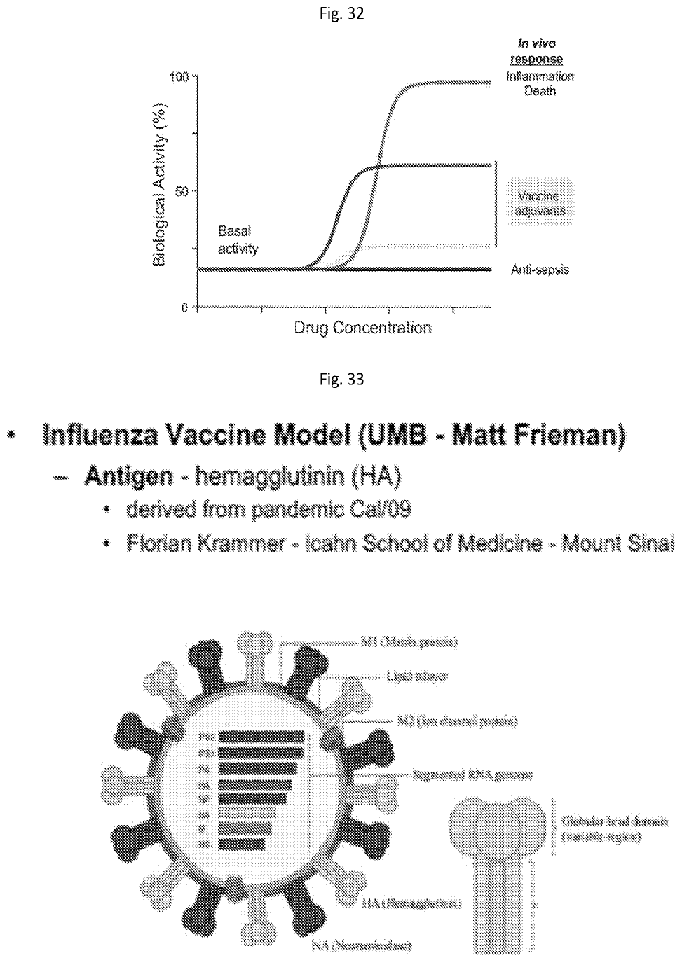

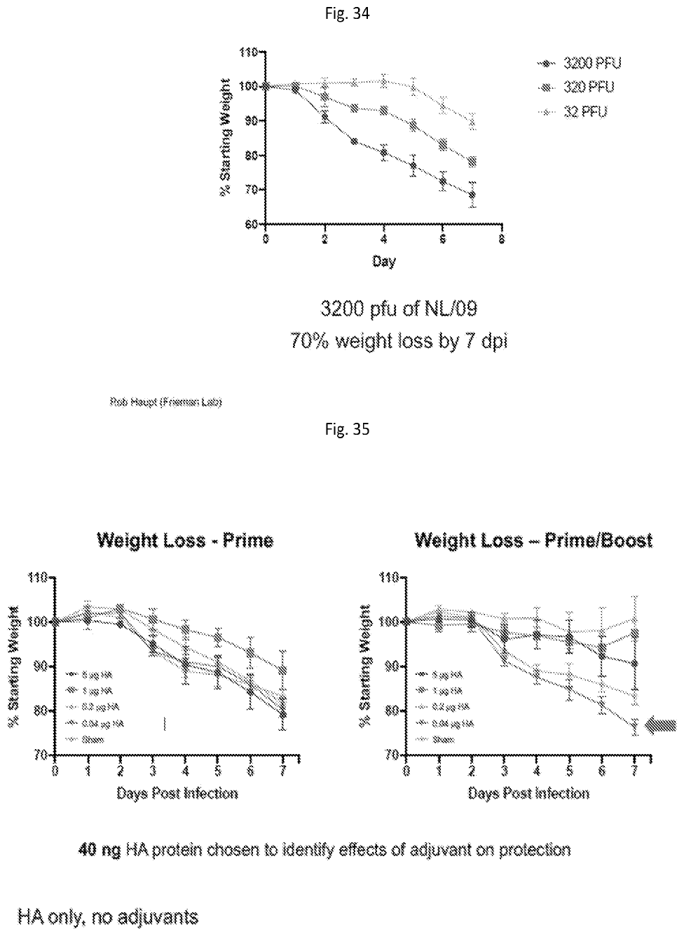

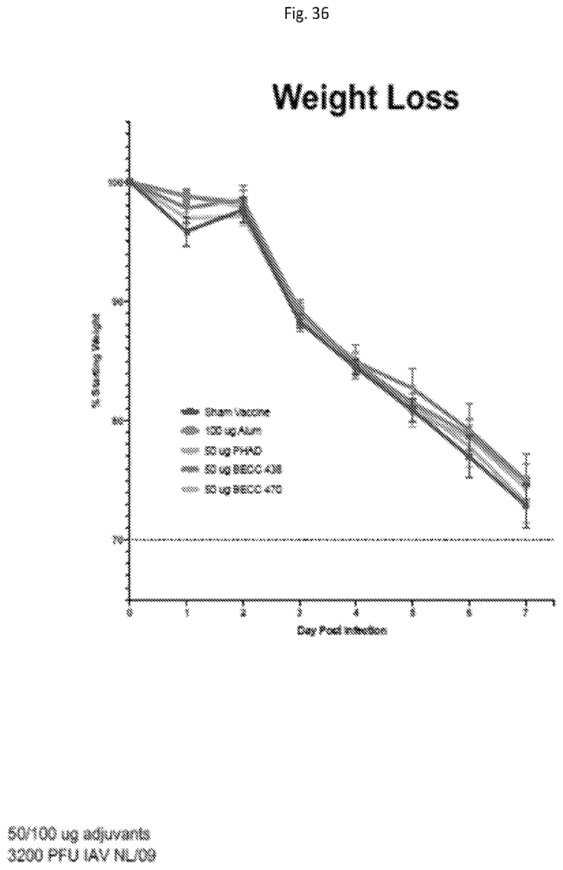

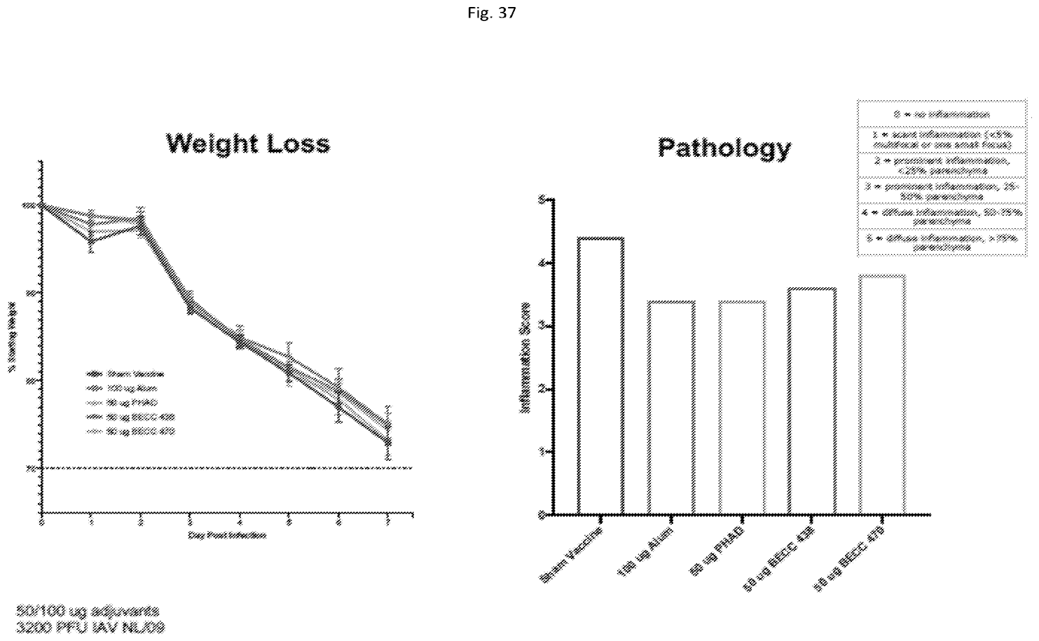

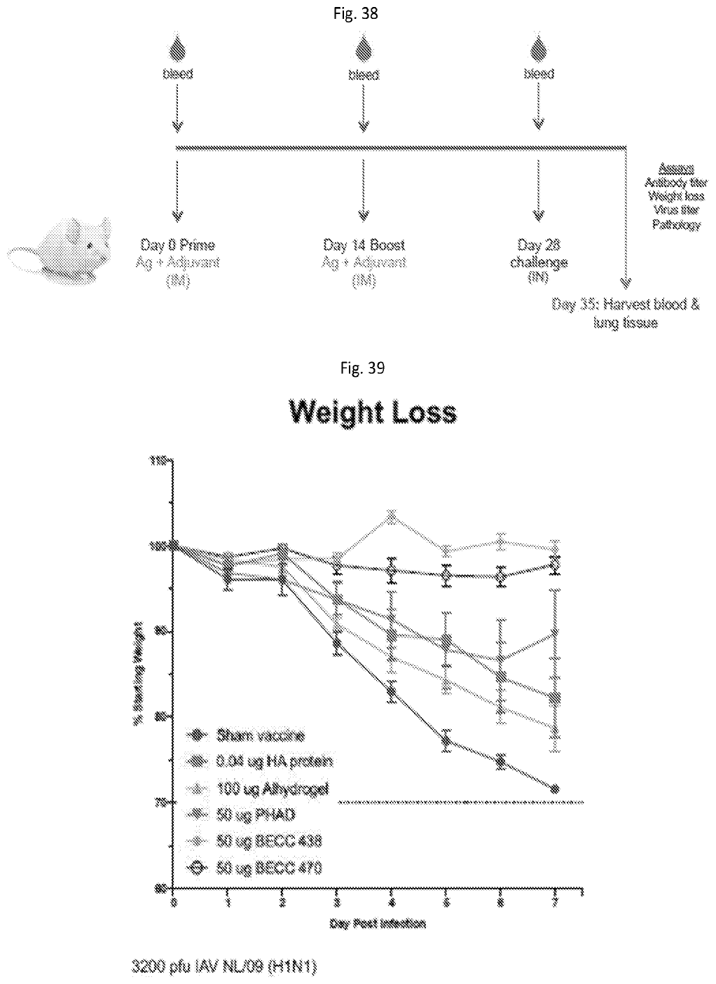

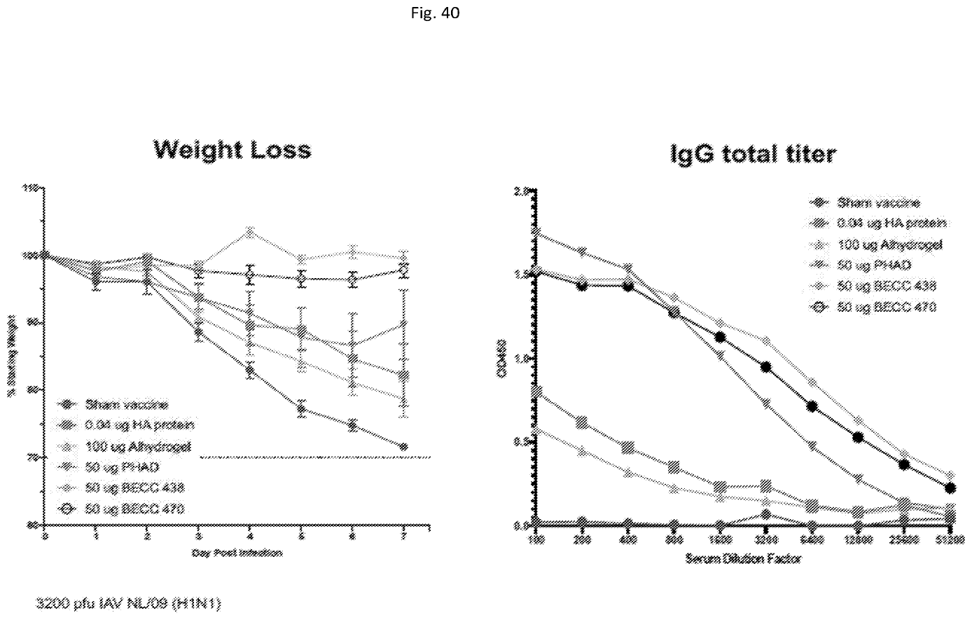

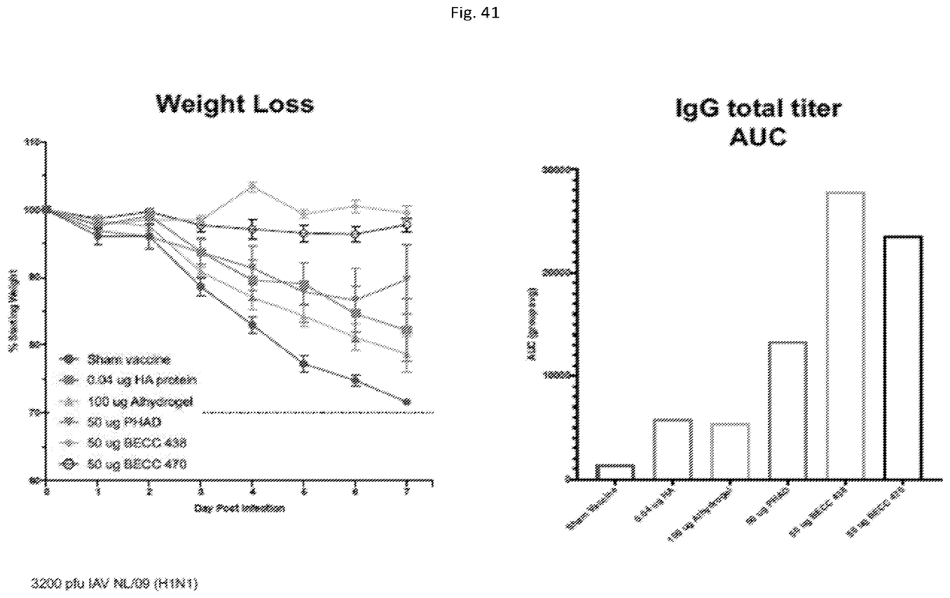

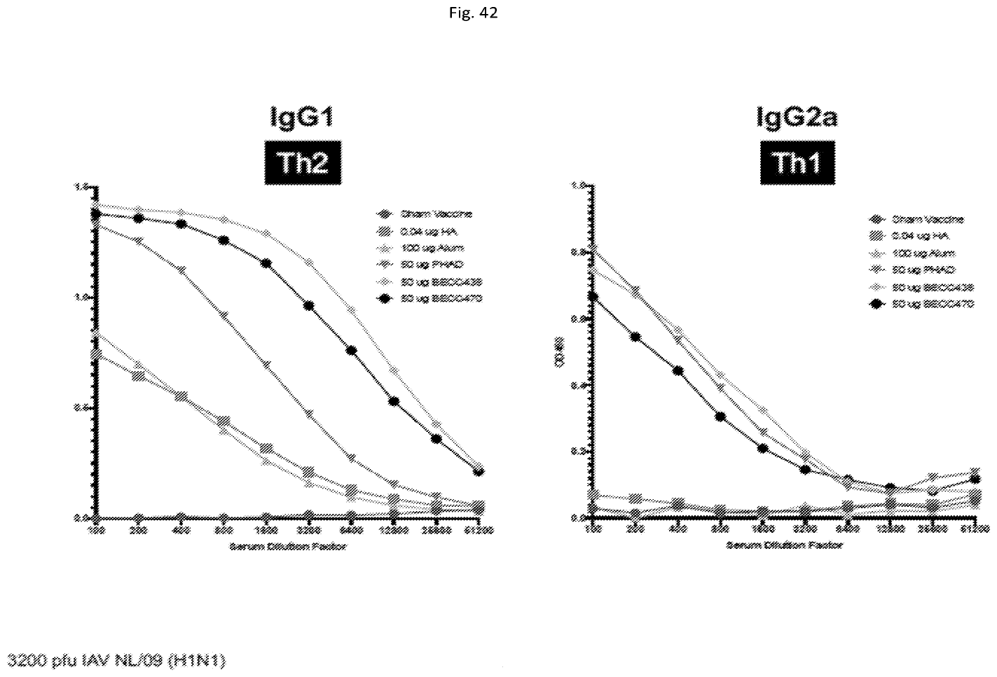

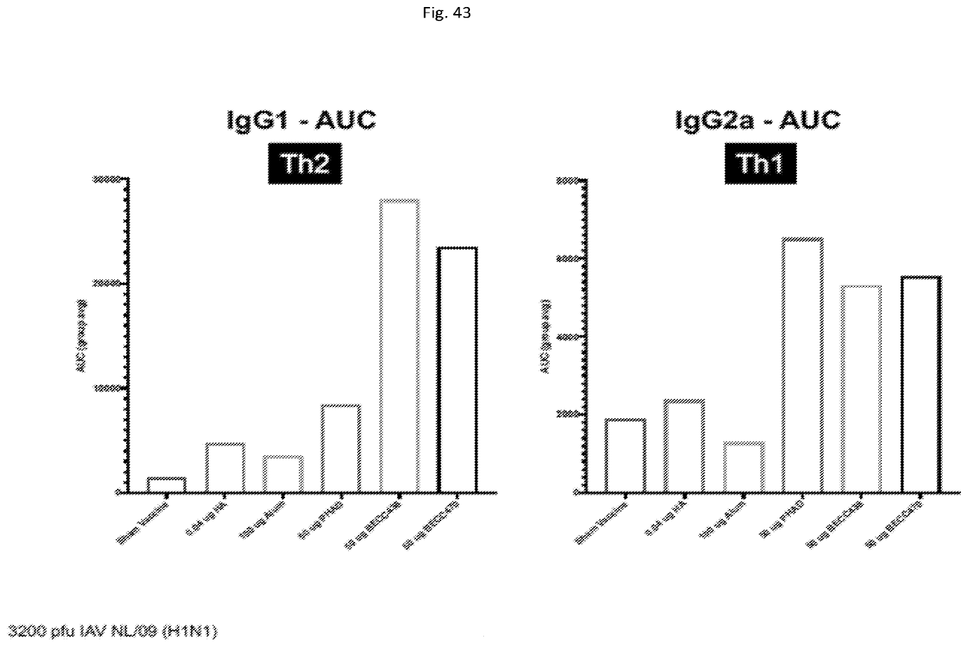

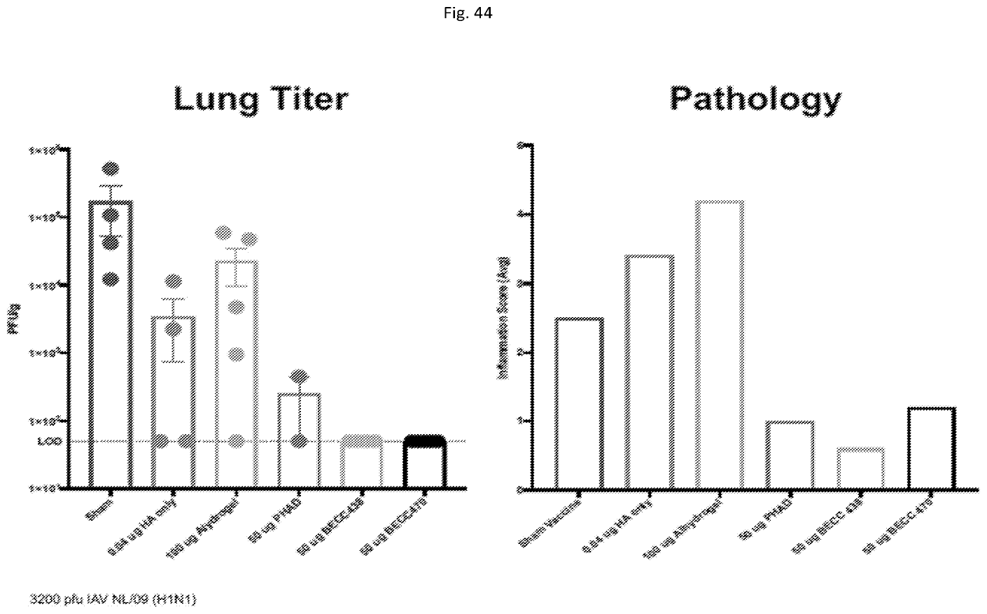

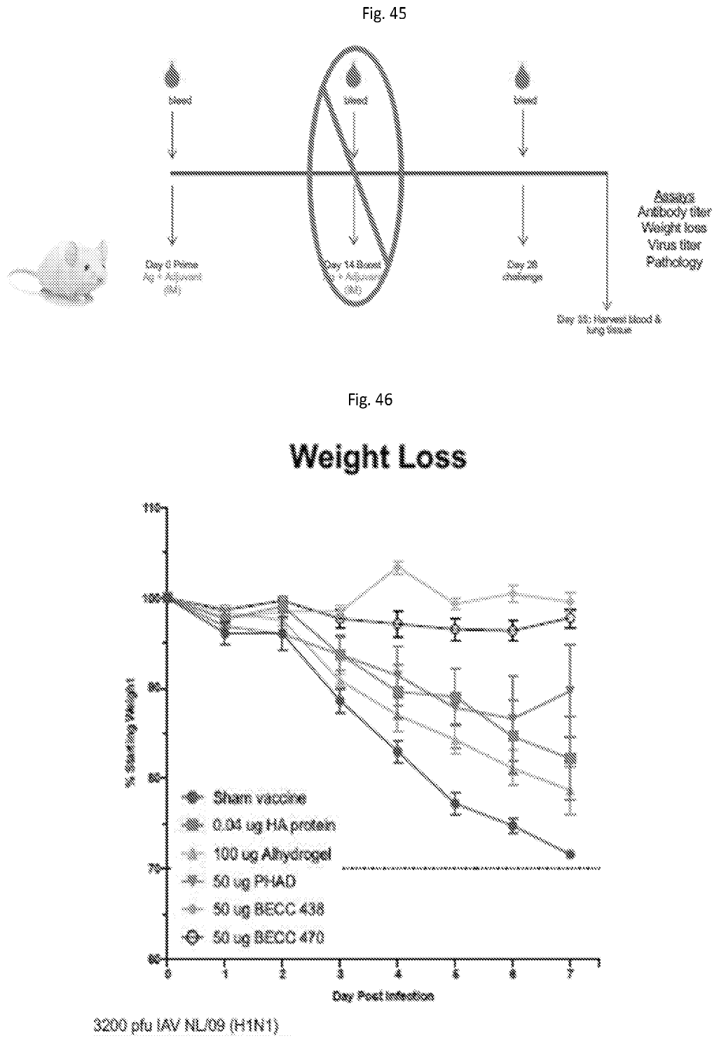

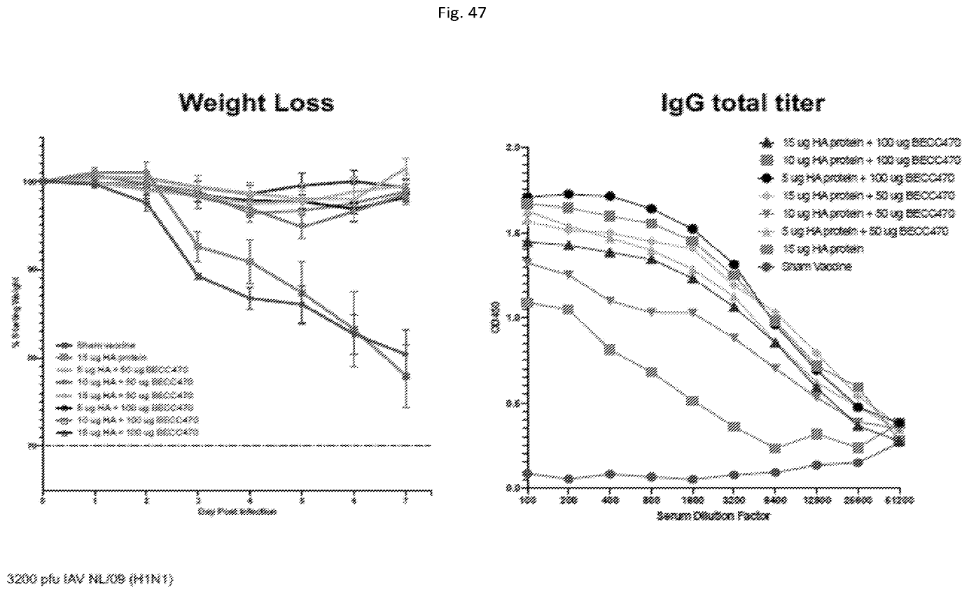

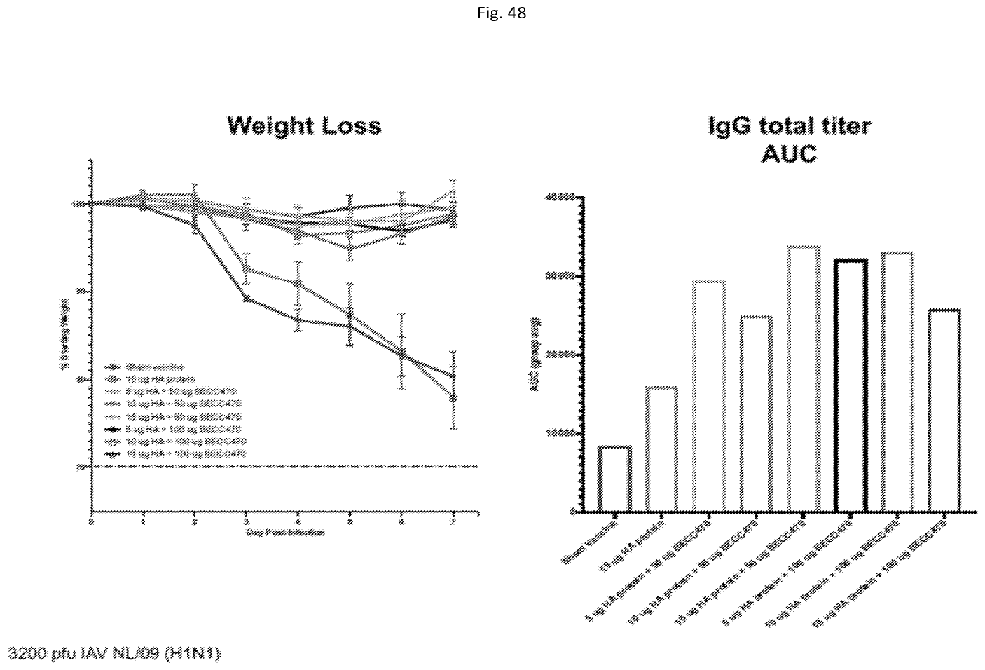

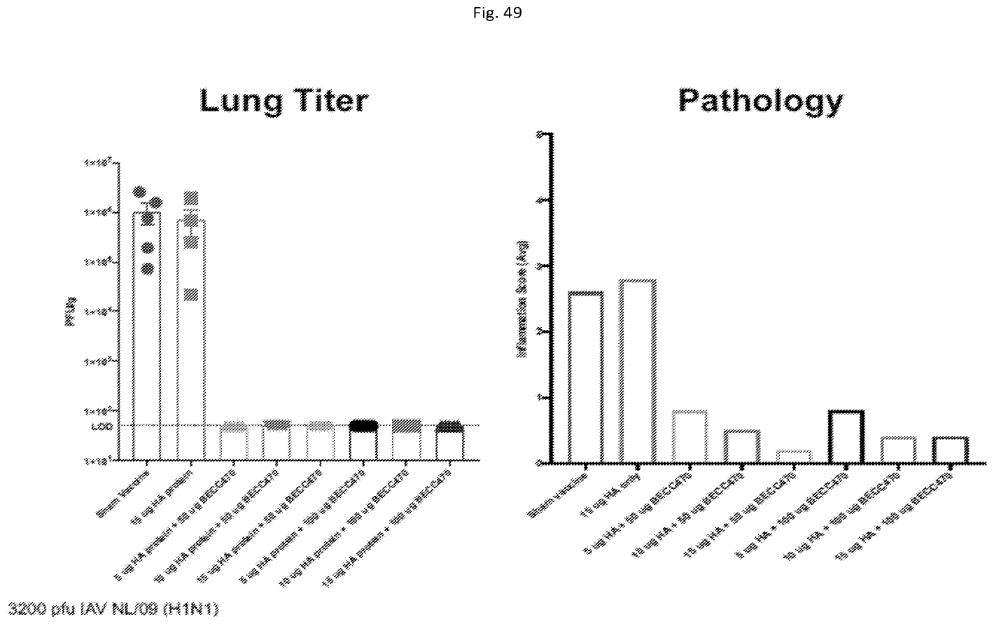

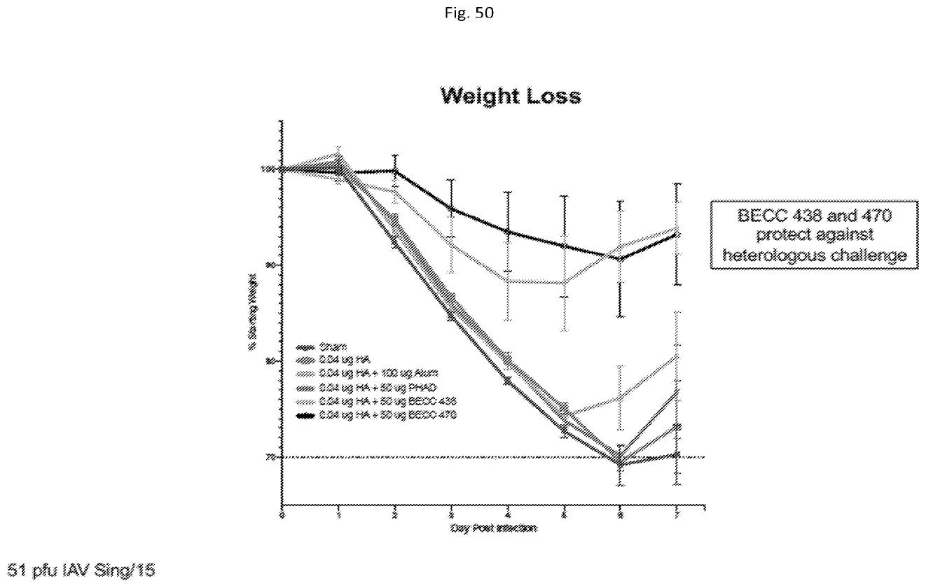

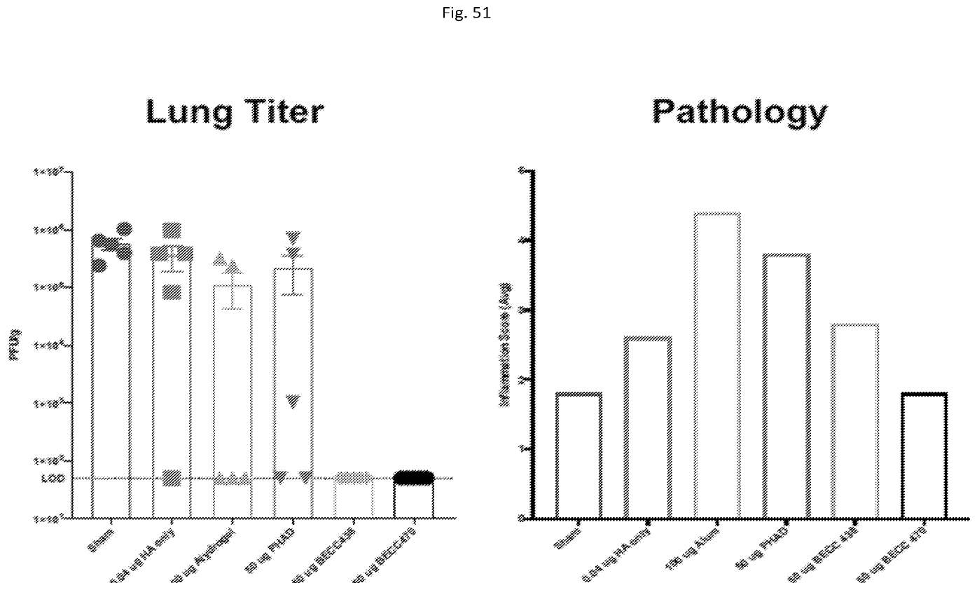

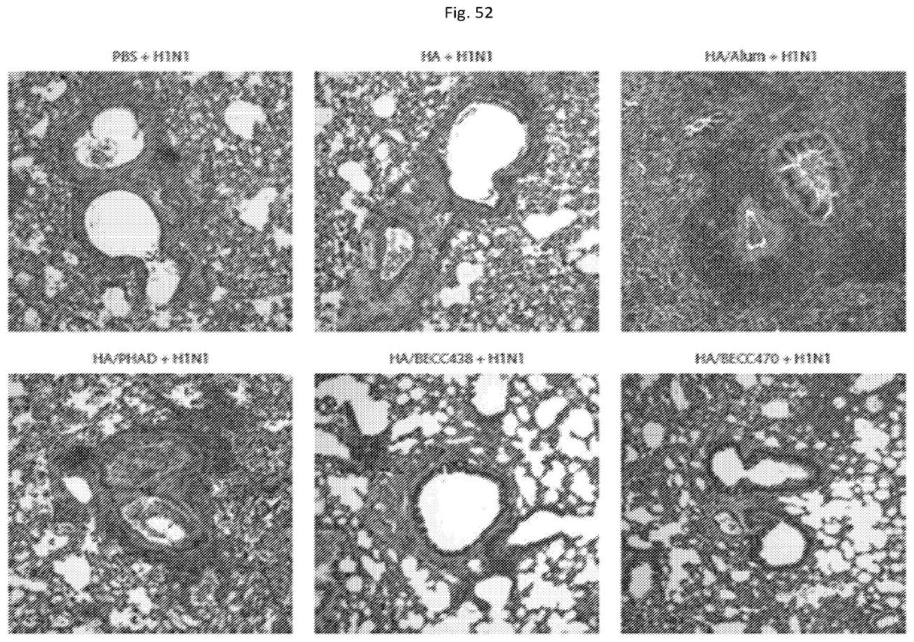

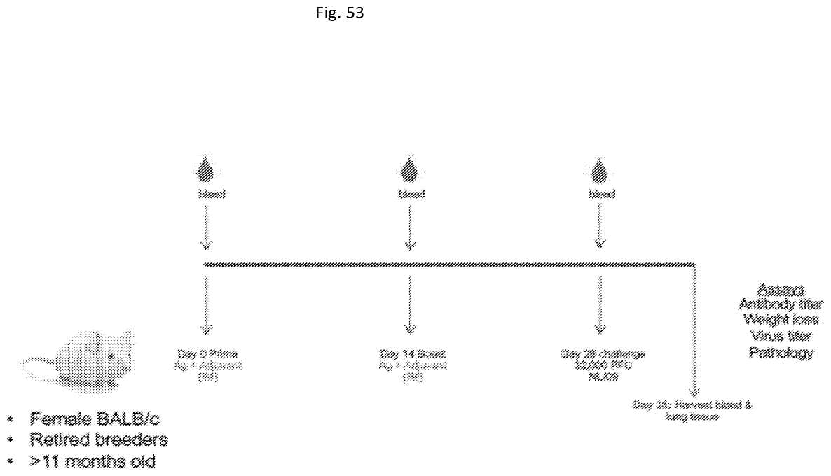

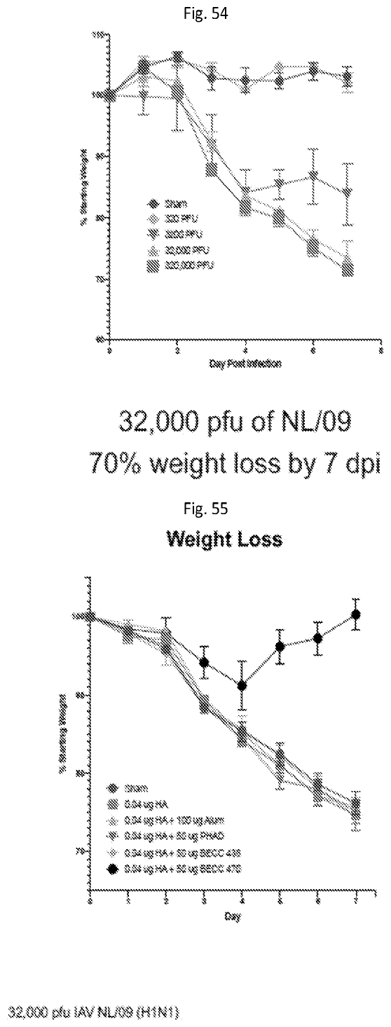

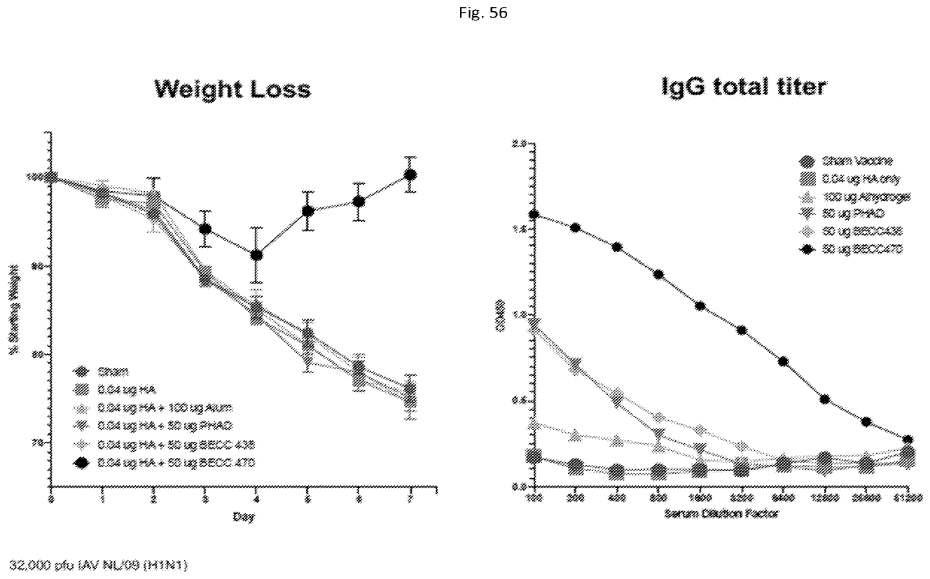

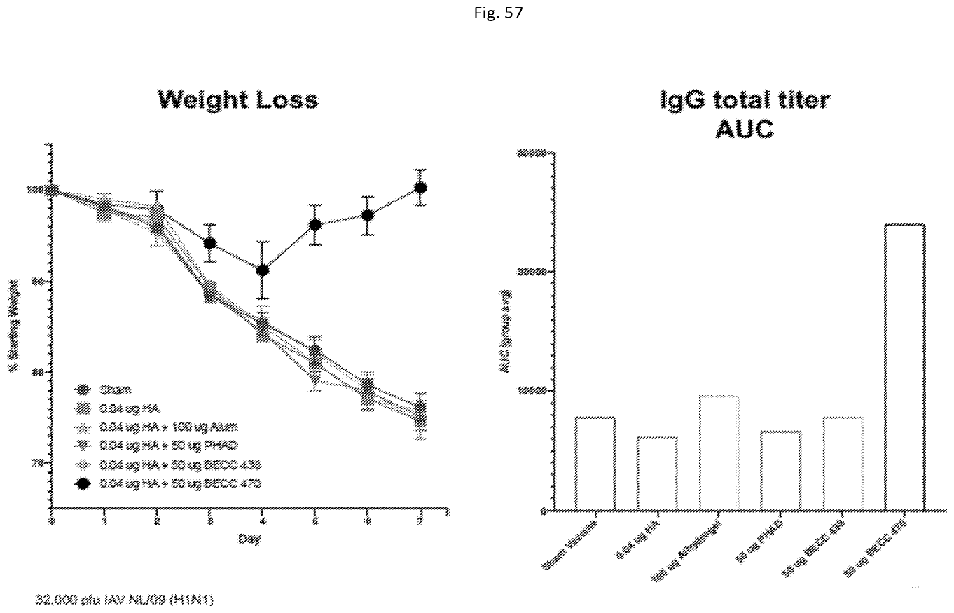

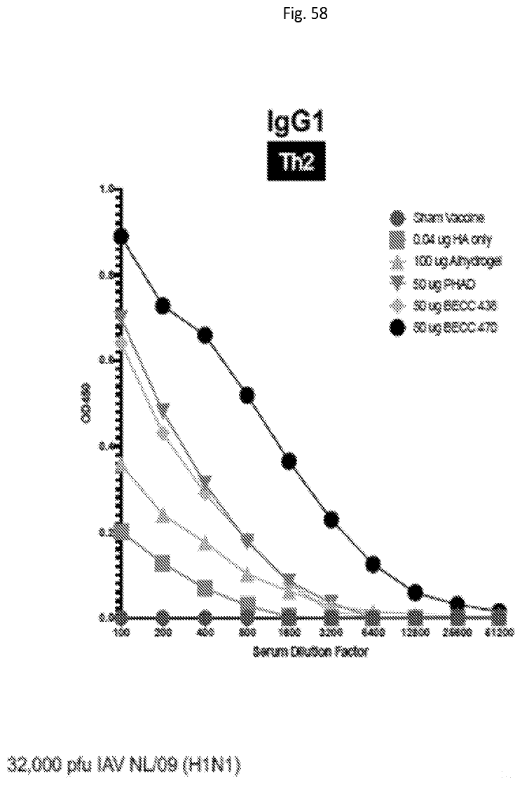

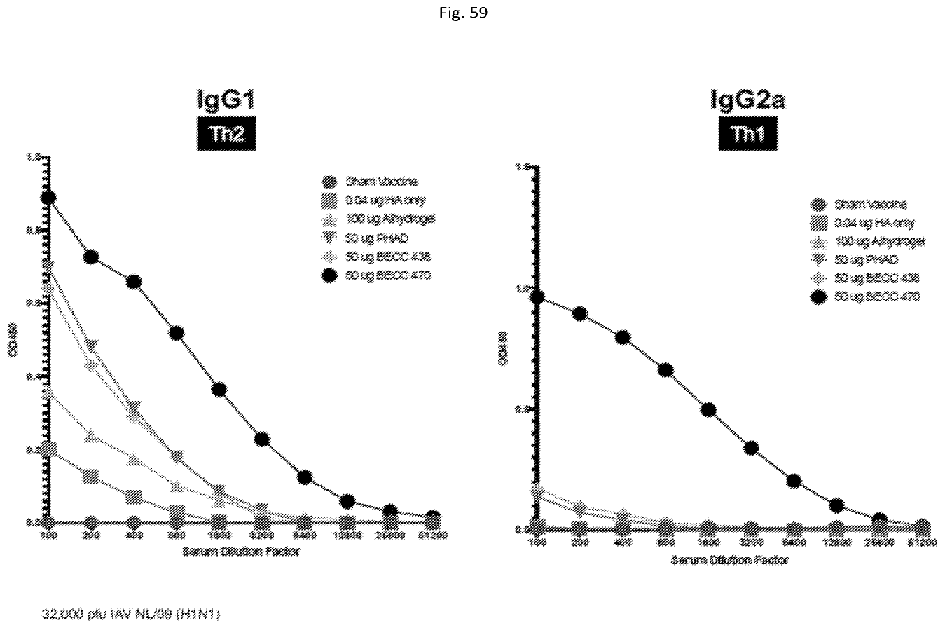

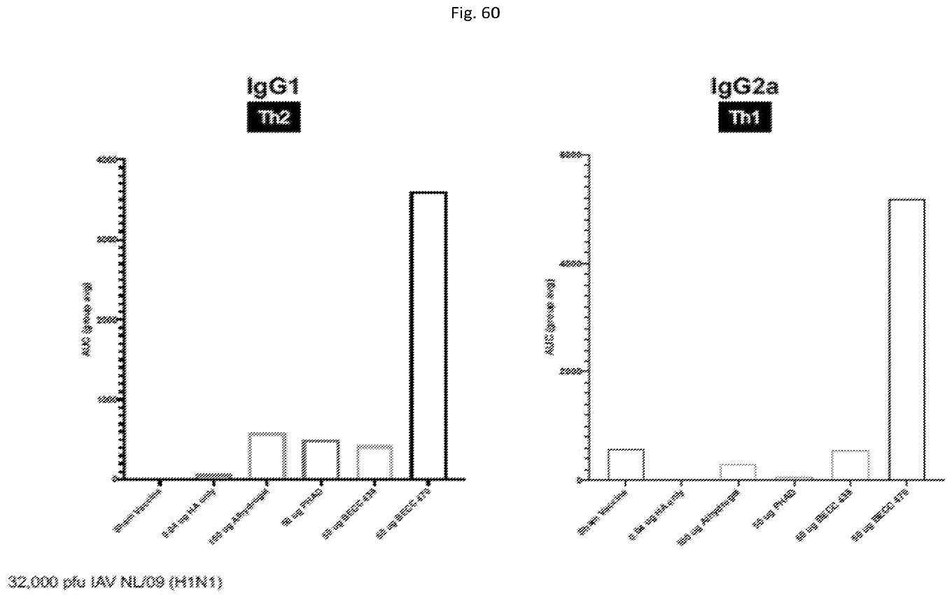

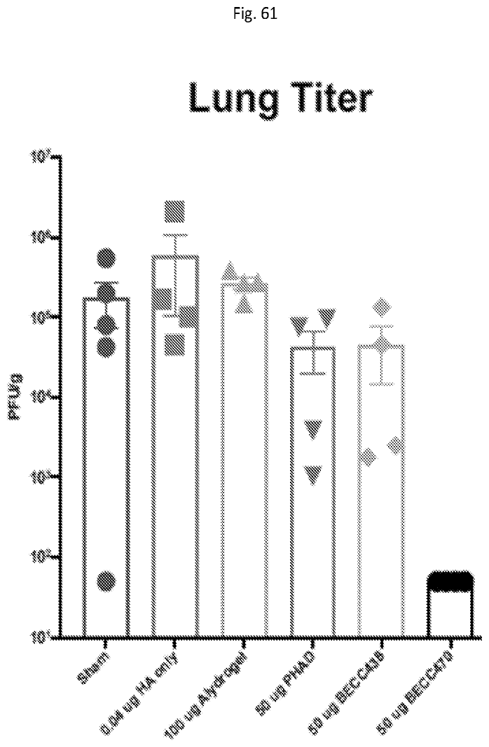

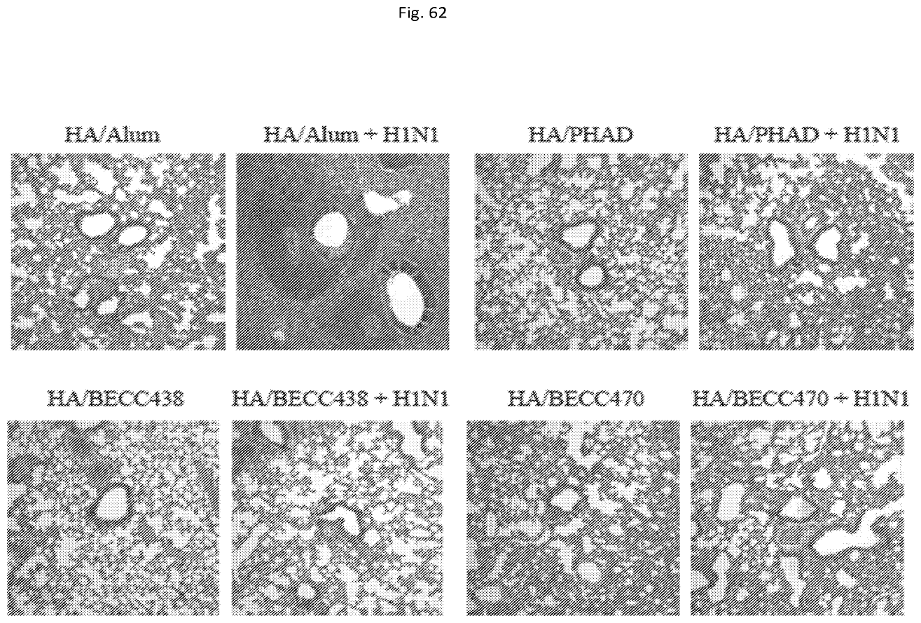

The most effective vaccines are able to induce long-lasting immunity that is characterized by strong memory T and/or B cell responses and are protective long after the initial pathogen insult. To determine whether the addition of BECC470 resulted in such long-lasting memory Ab responses, we monitored L1 ELISA Ab levels for 3 months after the third immunization at day 42. (FIG. 31A) shows that although total L1 Ab levels slowly wane over time, the boosted magnitude delivered by the presence of BECC470 compared to alum alone also continues over time, albeit gradually losing statistical significance. To further pursue the question of establishment of memory, the presence of T follicular helper (Tfh) cells in the spleen and popliteal lymph nodes (popLNs) was investigated, as Tfh cells are known to be critical drivers of memory B cell and plasma cell differentiation (23-24). At day 42, 2 weeks post third immunization, a substantial enhancement of the % of Tfh cells within the CD4 T cell compartment of both spleens and mLNs was observed in the BECC470/alum-adjuvanted mice, while the increase in Tfh cells in the alum alone group was significantly less robust (FIG. 31B-C). Therefore, the presence of BECC470 clearly improved the L1-specific B cell memory response, probably through robust proliferation of the Tfh subset within lymphoid centers.

TABLE 2
L2 RG1 epitope sequences compared between HPV
types. The amino acid sequences at positions
20-31 of L2 capsid for HPV16/18/39/6
were analyzed by BLOSUM62
comparison matrix for alignment.

| | RG1 20-31 | % identity | Score to |
|---|---|---|---|
| HPV type | a.a. sequence | to HPV16 | HPV16 |
| HPV16 | KTCKQAGTCPPD | n/a | n/a |
| HPV18 | KTCKQSGTCPPD | 91.70% | 70 |
| HPV39 | RTCKQSGTCPPD | 83.30% | 67 |
| HPV6 | QTCKLTGTCPPD | 75.00% | 58 |

In this study, both BECC compounds induced more robust humoral immune responses than did PHAD or Alhydrogel alone. Aluminum salt-based formulations like Alhydrogel are effective adjuvants widely approved for use in numerous vaccines including many delivered during childhood that target hepatitis A, hepatitis B, meningococcus B, pneumococcus, and DTaP (Diphtheria, tetanus, acellular pertussis) (reviewed in (45). The mechanism of action of alum-based preparations has long been unclear but recent findings point to the activation of the NLRP inflammasome in dendritic cells by uric acid crystals which then activates caspase-1 to cleave pro-IL-1β into active IL-1β for potential involvement (46-47) as well as the activation of the alternative pathway of the complement cascade (48). The combination of several TLR agonist adjuvant classes with aluminum salts has resulted in synergistic adjuvant activity, culminating in ASO4, a combination of Alhydrogel or Adju-Phos with the TLR4L MPLA, approved for the Cervarix and Fendrix vaccines (45). Comparison studies between Cervarix or the quadrivalent HPV vaccine Gardasil adjuvanted with AAHS have revealed that the presence of MPLA in Cervarix appears to lead to higher frequencies and magnitude of cross-neutralizing Abs (vs. HPV31/33/45/52/58) than does Gardasil (17), illuminating the benefit derived from the presence of a TLR4 agonist. In our study, we observed a similar potentiation of the adjuvant effects of Alhydrogel by the addition of BECC compounds with strong synergistic effects on several humoral immune response readouts. Efforts at vaccine optimization require novel adjuvant formulations to establish long-term protective immunity, exhibit little reactogenicity, demonstrate the capacity for large-scope scalability, and be relatively inexpensive (49). BECC lipid A is extracted from a biological source and is capable of rapid manufacturing scale-up in a more affordable method than that required for the chemical synthesis of compounds from the same TLR4L adjuvant class such as PHAD. Furthermore, unlike the original biologically derived MPLA, BECC molecules do not require post-extraction chemical modifications and can be lyophilized, which contributes to the high structural reliability and durability of this compound class.

Interestingly, in this murine model of HPV vaccination, BECC470 consistently outperformed BECC438 in adjuvant capabilities. This observed difference highlights the importance of structural changes of TLR4Ls to their ability to trigger signaling downstream of the TLR4/MD2 receptor complex. Although both MPLA and BECC470 are mono-phosphorylated, they differ by position of phosphate removal: 1′-position for MPLA and 4′-position for BECC470. And instead of the 2′ secondary 16-C acyl-chain addition with one unsaturation of BECC438, BECC470 has a 3′ secondary 12-C acyl-chain addition. This combination of phosphate removal and relative asymmetry of the BECC470 molecule may allow for formation and stabilization of a TLR4 receptor signaling complex that is particularly advantageous to promoting the induction of protective immunity without an overabundance of reactogenic responses (50).

While current HPV vaccines have enjoyed remarkable success at reducing rates of HPV infection and subsequent incidence of HPV-related cancers, there remains a need for improved vaccines that will lower COG, reduce the vaccine schedule, and amplify the humoral response. Our data indicate that supplementation of Alhydrogel with a BECC compound can optimize the activity and potency of the novel RG1-VLP vaccine by enhancing the magnitude of the Ab response, by allowing for VLP dose sparing, by accelerating the appearance of protective levels of neutralizing antibodies and thus allowing for a shorter vaccine schedule, and by promotion of longer-lasting Ab responses. Given the superiority displayed by BECC compounds over the standard TLR4L MPLA in terms of adjuvant activity, scale of synthesis, purity, and chemical stability, further investigation of adjuvanting RG1-VLPs with a combination adjuvant preparation of aluminum hydroxide+BECC compounds is warranted.

### LITERATURE (THIS EXAMPLE)

1. Serrano, B., L. Alemany, S. Tous, L. Bruni, G. M. Clifford, T. Weiss, F. X. Bosch, S. de Sanjose. 2012. Potential impact of a nine-valent vaccine in human papillomavirus related cervical disease. Infect Agent Cancer. 7:38.
2. Nguyen, H. P., M. K. Ramirez-Fort, P. L. Rady. 2014. The biology of human papillomaviruses. Curr Probl Dermatol. 45:19.
3. Jagu, S., K. Kwak, J. T. Schiller, D. R. Lowy, H. Kleanthous, K. Kalnin, C. Wang, H. K. Wang, L. T. Chow, W. K. Huh, K. S. Jaganathan, S. V. Chivukula, R. B. Roden. 2013. Phylogenetic considerations in designing a broadly protective multimeric L2 vaccine. J Virol. 87:6127.
4. Roden, R. B., W. H. t. Yutzy, R. Fallon, S. Inglis, D. R. Lowy, J. T. Schiller. 2000. Minor capsid protein of human genital papillomaviruses contains subdominant, cross-neutralizing epitopes. Virology. 270:254.
5. Roden, R. B., H. L. Greenstone, R. Kirnbauer, F. P. Booy, J. Jessie, D. R. Lowy, J. T. Schiller. 1996. In vitro generation and type-specific neutralization of a human papillomavirus type 16 virion pseudotype. J Virol. 70:5875.
6. Chen, X. S., R. L. Garcea, I. Goldberg, G. Casini, S. C. Harrison. 2000. Structure of small virus-like particles assembled from the L1 protein of human papillomavirus 16. Mol Cell. 5:557.
7. El Aliani, A., H. El Abid, Y. Kassal, M. Khyatti, M. Attaleb, M. M. Ennaji, M. El Mzibri. 2020. HPV16 L1 diversity and its potential impact on the vaccination-induced immunity. Gene. 747:144682.
8. Gambhira, R., B. Karanam, S. Jagu, J. N. Roberts, C. B. Buck, I. Bossis, H. Alphs, T. Culp, N. D. Christensen, R. B. Roden. 2007. A protective and broadly cross-neutralizing epitope of human papillomavirus L2. J Virol. 81:13927.
9. Chen, X., H. Liu, T. Zhang, Y. Liu, X. Xie, Z. Wang, X. Xu. 2014. A vaccine of L2 epitope repeats fused with a modified IgG1 Fc induced cross-neutralizing antibodies and protective immunity against divergent human papillomavirus types. PLoS One. 9:e95448.
10. Motavalli Khiavi, F., A. Arashkia, M. Golkar, M. Nasimi, F. Roohvand, K. Azadmanesh. 2018. A Dual-Type L2 11-88 Peptide from HPV Types 16/18 Formulated in Montanide ISA 720 Induced Strong and Balanced Th1/Th2 Immune Responses, Associated with High Titers of Broad Spectrum Cross-Reactive Antibodies in Vaccinated Mice. J Immunol Res. 2018:9464186.
11. Vujadinovic, M., S. Khan, K. Oosterhuis, T. G. Uil, K. Wunderlich, S. Damman, S. Boedhoe, A. Verwilligen, J. Knibbe, J. Serroyen, H. Schuitemaker, R. Zahn, G. Scheper, J. Custers, J. Vellinga. 2018. Adenovirus based HPV L2 vaccine induces broad cross-reactive humoral immune responses. Vaccine. 36:4462.
12. Nieto, K., M. Weghofer, P. Sehr, M. Ritter, S. Sedlmeier, B. Karanam, H. Seitz, M. Muller, M. Kellner, M. Horer, U. Michaelis, R. B. Roden, L. Gissmann, J. A. Kleinschmidt. 2012. Development of AAVLP(HPV16/31L2) particles as broadly protective HPV vaccine candidate. PLoS One. 7:e39741.
13. Jiang, R. T., C. Schellenbacher, B. Chackerian, R. B. Roden. 2016. Progress and prospects for L2-based human papillomavirus vaccines. Expert Rev Vaccines. 15:853.
14. Schellenbacher, C., R. Roden, R. Kirnbauer. 2009. Chimeric L1-L2 virus-like particles as potential broad-spectrum human papillomavirus vaccines. J Virol. 83:10085.
15. Schellenbacher, C., K. Kwak, D. Fink, S. Shafti-Keramat, B. Huber, C. Jindra, H. Faust, J. Dillner, R. B. S. Roden, R. Kirnbauer. 2013. Efficacy of RG1-VLP vaccination against infections with genital and cutaneous human papillomaviruses. J Invest Dermatol. 133:2706.
16. Nicoli, F., B. Mantelli, E. Gallerani, V. Telatin, I. Bonazzi, P. Marconi, R. Gavioli, L. Gabrielli, T. Lazzarotto, L. Barzon, G. Palu, A. A. Caputo. 2020. HPV-Specific Systemic Antibody Responses and Memory B Cells are Independently Maintained up to 6 Years and in a Vaccine-Specific Manner Following Immunization with Cervarix and Gardasil in Adolescent and Young Adult Women in Vaccination Programs in Italy. Vaccines (Basel). 8.
17. Mariz, F. C., N. Bender, D. Anantharaman, P. Basu, N. Bhatla, M. R. Pillai, P. R. Prabhu, R. Sankaranarayanan, T. Eriksson, M. Pawlita, K. Prager, P. Sehr, T. Waterboer, M. Muller, M. Lehtinen. 2020. Peak neutralizing and cross-neutralizing antibody levels to human papillomavirus types 6/16/18/31/33/45/52/58 induced by bivalent and quadrivalent HPV vaccines. NPJ Vaccines. 5:14.
18. Gregg, K. A., E. Harberts, F. M. Gardner, M. R. Pelletier, C. Cayatte, L. Yu, M. P. McCarthy, J. D. Marshall, R. K. Ernst. 2017. Rationally Designed TLR4 Ligands for Vaccine Adjuvant Discovery. mBio. 8.
19. Gregg, K. A., E. Harberts, F. M. Gardner, M. R. Pelletier, C. Cayatte, L. Yu, M. P. McCarthy, J. D. Marshall, R. K. Ernst. 2018. A lipid A-based TLR4 mimetic effectively adjuvants a Yersinia pestis rF-V1 subunit vaccine in a murine challenge model. Vaccine. 36:4023.
20. Wang, J. W., S. Jagu, K. Kwak, C. Wang, S. Peng, R. Kirnbauer, R. B. Roden. 2014. Preparation and properties of a papillomavirus infectious intermediate and its utility for neutralization studies. Virology. 449:304.
21. Coler, R. N., S. Bertholet, M. Moutaftsi, J. A. Guderian, H. P. Windish, S. L. Baldwin, E. M. Laughlin, M. S. Duthie, C. B. Fox, D. Carter, M. Friede, T. S. Vedvick, S. G. Reed. 2011. Development and characterization of synthetic glucopyranosyl lipid adjuvant system as a vaccine adjuvant. PLoS One. 6:e16333.
22. Wang, J. W., K. Matsui, Y. Pan, K. Kwak, S. Peng, T. Kemp, L. Pinto, R. B. Roden. 2015. Production of Furin-Cleaved Papillomavirus Pseudovirions and Their Use for In Vitro Neutralization Assays of L1- or L2-Specific Antibodies. Curr Protoc Microbiol. 38:14B 5 1.
23. Crotty, S. 2011. Follicular helper CD4 T cells (TFH). Annu Rev Immunol. 29:621.
24. Matsui, K., J. W. Adelsberger, T. J. Kemp, M. W. Baseler, J. E. Ledgerwood, L. A. Pinto. 2015. Circulating CXCR5(+)CD4(+) T Follicular-Like Helper Cell and Memory B Cell Responses to Human Papillomavirus Vaccines. PLoS One. 10:e0137195.
25. Pastrana, D. V., R. Gambhira, C. B. Buck, Y. Y. Pang, C. D. Thompson, T. D. Culp, N. D. Christensen, D. R. Lowy, J. T. Schiller, R. B. Roden. 2005. Cross-neutralization of cutaneous and mucosal Papillomavirus types with anti-sera to the amino terminus of L2. Virology. 337:365.
26. Sapp, M., M. Bienkowska-Haba. 2009. Viral entry mechanisms: human papillomavirus and a long journey from extracellular matrix to the nucleus. FEBS J. 276:7206.
27. Rubio, I., H. Seitz, E. Canali, P. Sehr, A. Bolchi, M. Tommasino, S. Ottonello, M. Muller. 2011. The N-terminal region of the human papillomavirus L2 protein contains overlapping binding sites for neutralizing, cross-neutralizing and non-neutralizing antibodies. Virology. 409:348.
28. Chakravarty, J., S. Kumar, S. Trivedi, V. K. Rai, A. Singh, J. A. Ashman, E. M. Laughlin, R. N. Coler, S. J. Kahn, A. M. Beckmann, K. D. Cowgill, S. G. Reed, S. Sundar, F. M. Piazza. 2011. A clinical trial to evaluate the safety and immunogenicity of the LEISH-F1+MPL-SE vaccine for use in the prevention of visceral leishmaniasis. Vaccine. 29:3531.
29. Carter, D., N. van Hoeven, S. Baldwin, Y. Levin, E. Kochba, A. Magill, N. Charland, N. Landry, K. Nu, A. Frevol, J. Ashman, Z. K. Sagawa, A. M. Beckmann, S. G. Reed. 2018. The adjuvant GLA-AF enhances human intradermal vaccine responses. Sci Adv. 4:eaas9930.
30. D'Addario, M., S. Redmond, P. Scott, D. Egli-Gany, A. X. Riveros-Balta, A. M. Henao Restrepo, N. Low. 2017. Two-dose schedules for human papillomavirus vaccine: Systematic review and meta-analysis. Vaccine. 35:2892.
31. Pasmans, H., T. M. Schurink-Van′t Klooster, M. J. M. Bogaard, D. M. van Rooijen, H. E. de Melker, M. J. P. Welters, S. H. van der Burg, F. R. M. van der Klis, A. M. Buisman. 2019. Long-term HPV-specific immune response after one versus two and three doses of bivalent HPV vaccination in Dutch girls. Vaccine. 37:7280.
32. Brotherton, J. M. 2018. Human papillomavirus vaccination update: Nonavalent vaccine and the two-dose schedule. Aust J Gen Pract. 47:417.
33. Kreimer, A. R., J. N. Sampson, C. Porras, J. T. Schiller, T. Kemp, R. Herrero, S. Wagner, J. Boland, J. Schussler, D. R. Lowy, S. Chanock, D. Roberson, M. S. Sierra, S. H. Tsang, M. Schiffman, A. C. Rodriguez, B. Cortes, M. H. Gail, A. Hildesheim, P. Gonzalez, L. A. Pinto, H. P. V. V. T. G. Costa Rica. 2020. Evaluation of durability of a single-dose of the bivalent HPV vaccine: the CVT Trial. J Natl Cancer Inst.
34. Brotherton, J. M., A. Budd, C. Rompotis, N. Bartlett, M. J. Malloy, R. L. Andersen, K. A. Coulter, P. W. Couvee, N. Steel, G. H. Ward, M. Saville. 2019. Is one dose of human papillomavirus vaccine as effective as three?: A national cohort analysis. Papillomavirus Res. 8:100177.
35. Quan, F. S., D. G. Yoo, J. M. Song, J. D. Clements, R. W. Compans, S. M. Kang. 2009. Kinetics of immune responses to influenza virus-like particles and dose-dependence of protection with a single vaccination. J Virol. 83:4489.
36. Dietrich, J., L. V. Andreasen, P. Andersen, E. M. Agger. 2014. Inducing dose sparing with inactivated polio virus formulated in adjuvant CAF01. PLoS One. 9:e100879.
37. Oleszycka, E., S. McCluskey, F. A. Sharp, N. Munoz-Wolf, E. Hams, A. L. Gorman, P. G. Fallon, E. C. Lavelle. 2018. The vaccine adjuvant alum promotes IL-10 production that suppresses Th1 responses. Eur J Immunol. 48:705.
38. Dupuy, C., D. Buzoni-Gatel, A. Touze, P. Le Cann, D. Bout, P. Coursaget. 1997. Cell mediated immunity induced in mice by HPV 16 L1 virus-like particles. Microb Pathog. 22:219.
39. Yokomine, M., S. Matsueda, K. Kawano, T. Sasada, A. Fukui, T. Yamashita, N. Komatsu, S. Shichijo, K. Tasaki, K. Matsukuma, K. Itoh, T. Kamura, K. Ushijima. 2017. Enhancement of humoral and cell mediated immune response to HPV16 L1-derived peptides subsequent to vaccination with prophylactic bivalent HPV L1 virus-like particle vaccine in healthy females. Exp Ther Med. 13:1500.
40. Pinto, L. A., J. Edwards, P. E. Castle, C. D. Harro, D. R. Lowy, J. T. Schiller, D. Wallace, W. Kopp, J. W. Adelsberger, M. W. Baseler, J. A. Berzofsky, A. Hildesheim. 2003. Cellular immune responses to human papillomavirus (HPV)-16 L1 in healthy volunteers immunized with recombinant HPV-16 L1 virus-like particles. J Infect Dis. 188:327.
41. Steele, J. C., C. H. Mann, S. Rookes, T. Rollason, D. Murphy, M. G. Freeth, P. H. Gallimore, S. Roberts. 2005. T-cell responses to human papillomavirus type 16 among women with different grades of cervical neoplasia. Br J Cancer. 93:248.
42. Roden, R. B. S., P. L. Stern. 2018. Opportunities and challenges for human papillomavirus vaccination in cancer. Nat Rev Cancer. 18:240.
43. Smith, K. M., L. Pottage, E. R. Thomas, A. J. Leishman, T. N. Doig, D. Xu, F. Y. Liew, P. Garside. 2000. Th1 and Th2 CD4+ T cells provide help for B cell clonal expansion and antibody synthesis in a similar manner in vivo. J Immunol. 165:3136.
44. Wang, J. W., W. H. Wu, T. C. Huang, M. Wong, K. Kwak, K. Ozato, C. F. Hung, R. B. S. Roden. 2018. Roles of Fc Domain and Exudation in L2 Antibody-Mediated Protection against Human Papillomavirus. J Virol. 92.
45. HogenEsch, H., D. T. O'Hagan, C. B. Fox. 2018. Optimizing the utilization of aluminum adjuvants in vaccines: you might just get what you want. NPJ Vaccines. 3:51.
46. Kool, M., K. Fierens, B. N. Lambrecht. 2012. Alum adjuvant: some of the tricks of the oldest adjuvant. J Med Microbiol. 61:927.
47. Fierens, K., M. Kool. 2012. The mechanism of adjuvanticity of aluminium-containing formulas. Curr Pharm Des. 18:2305.
48. Guven, E., K. Duus, I. Laursen, P. Hojrup, G. Houen. 2013. Aluminum hydroxide adjuvant differentially activates the three complement pathways with major involvement of the alternative pathway. PLoS One. 8:e74445.
49. Nanishi, E., D. J. Dowling, O. Levy. 2020. Toward precision adjuvants: optimizing science and safety. Curr Opin Pediatr. 32:125.
50. Giordano, N. P., M. B. Cian, Z. D. Dalebroux. 2020. Outermembrane lipid secretion and the innate immune response to Gram-negative bacteria. Infect Immun.

What is claimed is:

1. A pharmaceutical composition capable of inducing an immune response in a subject, comprising an effective amount of a viral immunogen and an adjuvant, wherein the adjuvant comprises a lipid A mimetic molecule selected from the group consisting of: 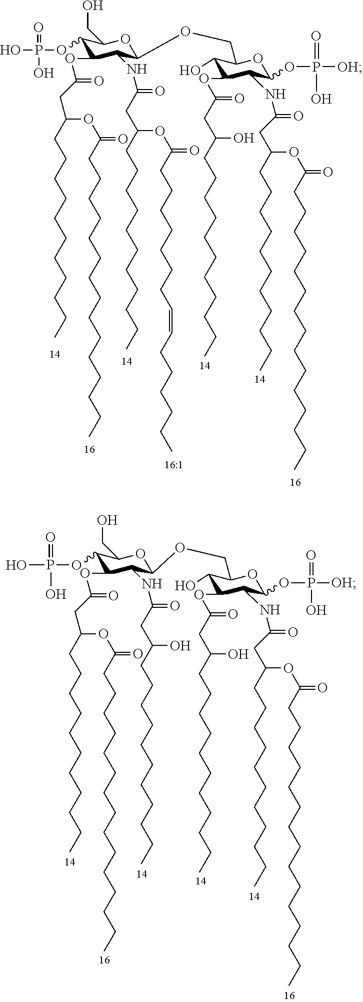 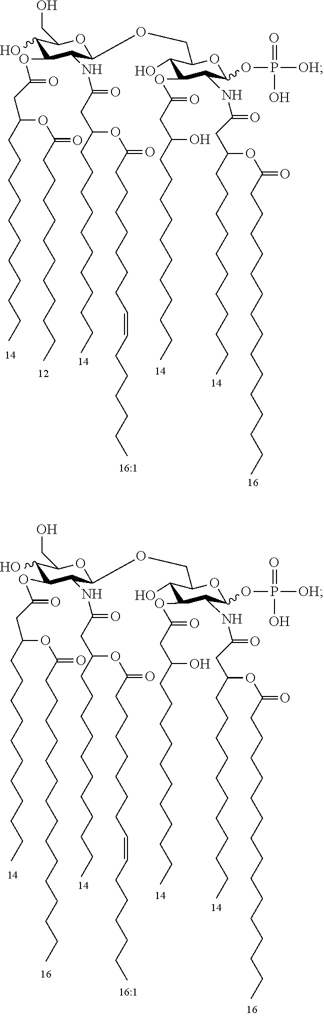 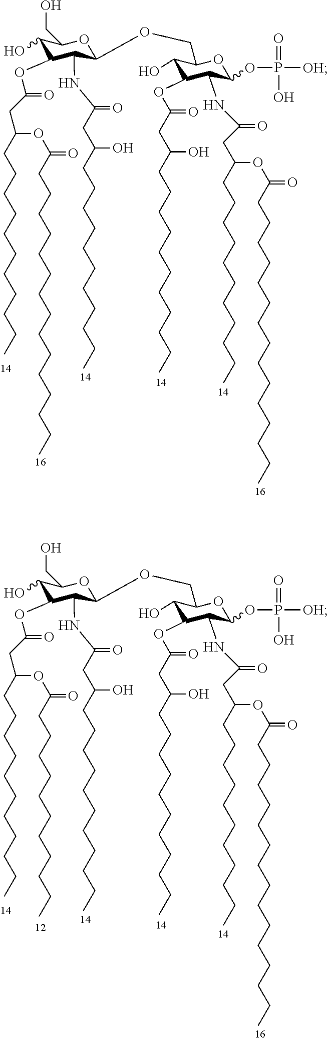
and combinations thereof.

2. The composition of claim 1, wherein the subject is a human.

3. The composition of any of claims 1-2, wherein the viral immunogen is from an influenza virus.

4. The composition of any of claims 1-3, wherein the viral immunogen is from an influenza A virus.

5. The composition of any of claims 1-3, wherein the viral immunogen is from an influenza B virus.

6. The composition of claims 1-2, wherein the viral immunogen is from respiratory syncytial virus (RSV).

7. The composition of claims 1-2, wherein the viral immunogen is from a human papillomavirus (HPV).

8. The composition of any of claims 1-7, wherein the viral immunogen comprises a polypeptide antigen or an antigenic fragment thereof.

9. The composition of claim 8, wherein the antigen comprises influenza hemagglutinin (HA) protein or an antigenic fragment thereof.

10. The composition of any of claims 1-9, further comprising a pharmaceutically acceptable carrier.

11. A method of inducing an immune response in a subject, comprising administering to the subject a pharmaceutical composition comprising an effective amount of a viral immunogen and an adjuvant, wherein the adjuvant comprises a lipid A mimetic molecule selected from the group consisting of: 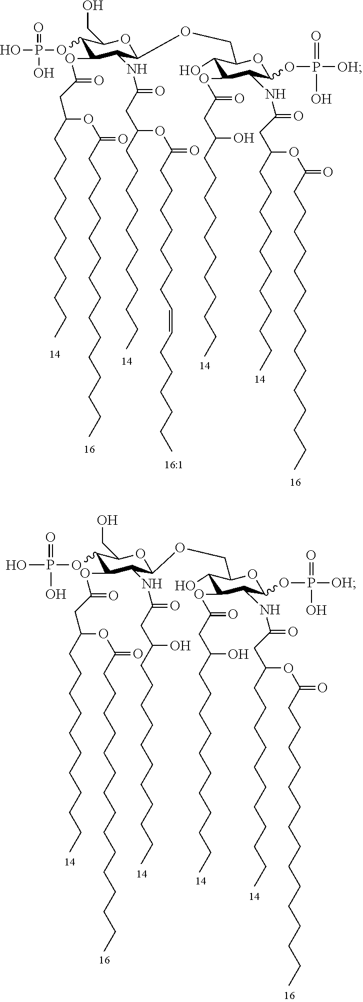 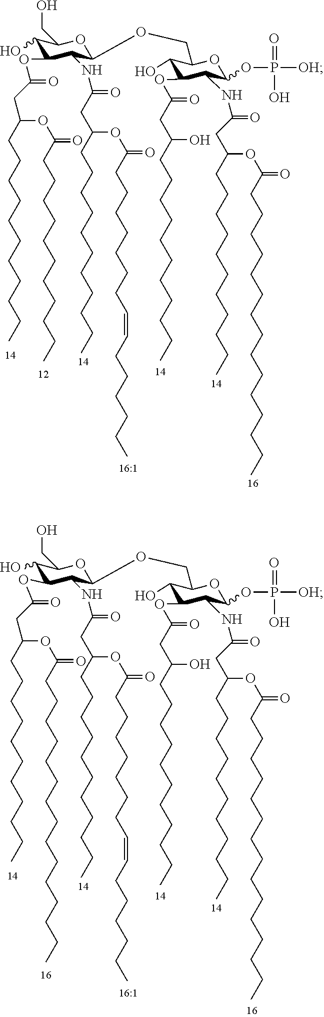 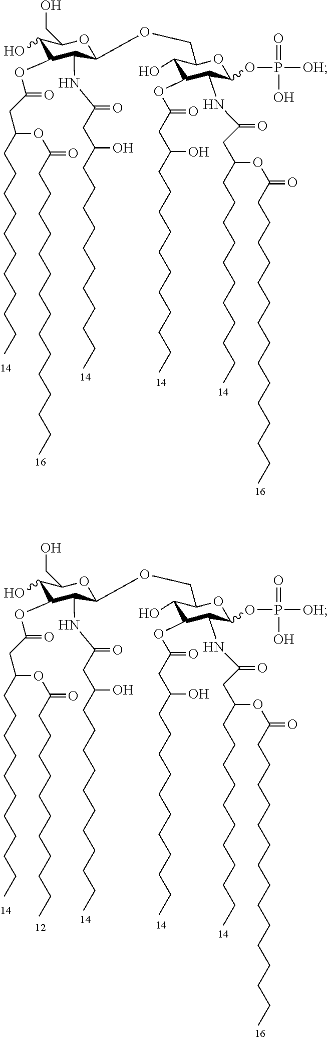
and combinations thereof.

12. The method of claim 11, wherein the subject is a mammal.

13. The method of any of claims 11-12, wherein the subject is a human.

14. The method of any of claims 11-13, wherein the viral immunogen is from an influenza A virus.

15. The method of any of claims 11-13, wherein the viral immunogen is from an influenza B virus.

16. The method of any of claims 11-13, wherein the viral immunogen is from a respiratory syncytial virus (RSV).

17. The method of any of claims 11-13, wherein the viral immunogen is from a human papillomavirus (HPV).

18. The method of any of claims 11-17, wherein the pharmaceutical composition is administered by intramuscular injection.

19. The method of any of claims 11-18, wherein the immunogen is selected from the group consisting of a split virus, a subunit antigen, an inactivated whole virus, a live attenuated virus, and combinations thereof.

20. The method of any of claims 11-19, wherein the subject is administered the composition only once.

21. The method of any of claims 11-19, wherein the subject is administered a first dose of the composition as a prime, followed by administration of one or more additional boost administrations.

22. The method of any of claims 11-21, wherein the method reduces lethality of a secondary bacterial infection.

23. The method of any of claims 11-22, wherein the effective amount of the viral immunogen administered is from about 50 ng to about 1.0 mg per kg of body weight of the subject.

24. The method of any of claims 11-22, wherein the effective amount of the viral immunogen administered is from about 15 μg to about 1.9 mg per kg of body weight of the mammal.

25. The method of any of claims 11-24, wherein the adjuvant comprises a lipid A mimetic molecule selected from the group consisting of:

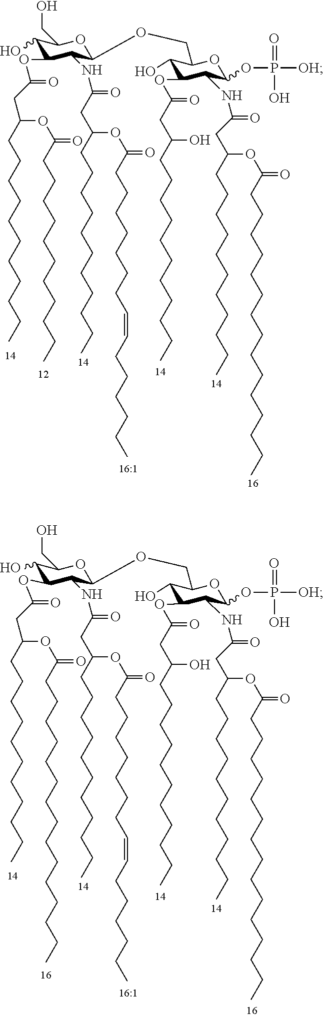

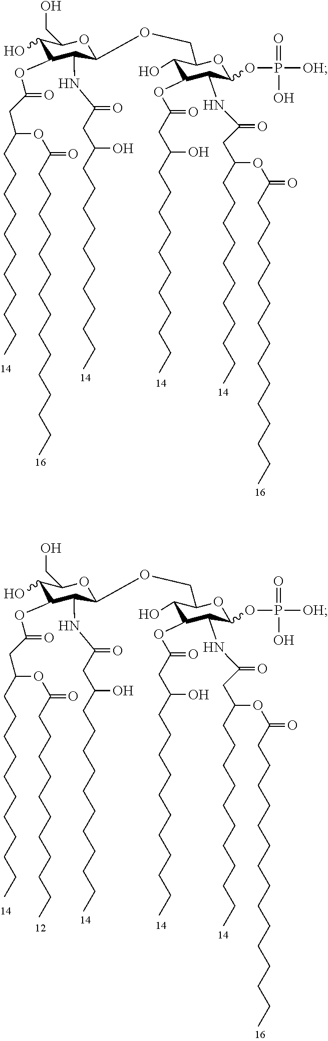

26. [symbol] and combinations thereof.

27. The method of claim 25, wherein the viral immunogen is from influenza.

28. The method of any of claim 25 or 26, wherein the viral immunogen comprises hemagglutinin or any antigenic fragment thereof.

29. The method of any of claim 25 or 27, wherein the subject is a human.

30. The method of claim 28, wherein the subject is 55 years old or greater.
